(19) United States
(12) Patent Application Publication
(10) Pub. No.: US 2025/0289884 A1
(43) Pub. Date: Sep. 18, 2025

(54) ANTI-NOTCH3 ANTIBODIES

(71) Applicant: AVEO Pharmaceuticals, Inc., Boston, MA (US)

(72) Inventors: Heidi Okamura, Brookline, MA (US); Sandra Abbott, Boston, MA (US); Alisa C. Bell, Bedford, MA (US); Kelly Kreuter, Arvada, CO (US); Ronan O'Hagan, Arlington, VA (US); Samantha Perino, Brighton, MA (US); Hamid Tissire, Abington, MA (US); William M. Winston, JR., Marlborough, MA (US); Jeno Gyuris, Lincoln, MA (US)

(21) Appl. No.: 19/063,993

(22) Filed: Feb. 26, 2025

Related U.S. Application Data
(63) Continuation of application No. 16/992,897, filed on Aug. 13, 2020, now Pat. No. 12,269,877.

(51) Int. Cl. C07K 16/28; A61K 39/00; A61K 39/395; C12N 5/10; C12N 5/12; C12N 5/16; C12N 15/09; C12N 15/63

(52) U.S. Cl. CPC C07K 16/28; A61K 39/3955; C12N 15/09; C12N 15/63; A61K 2039/505; C07K 16/2863; C07K 2317/14; C07K 2317/24; C07K 2317/515; C07K 2317/56; C07K 2317/565; C07K 2317/76; C07K 2317/92; C12N 5/10; C12N 5/12; C12N 5/16

(57) ABSTRACT

Monoclonal antibodies that bind and inhibit activation of human Notch3 are disclosed. The antibodies can be used to treat cell proliferative diseases and disorders, including certain forms of cancer, associated with activation of Notch3.

### CROSS-REFERENCE TO RELATED APPLICATIONS

This application is a continuation of U.S. patent application Ser. No. 16/992,897 filed Aug. 13, 2020, which is a continuation of U.S. patent application Ser. No. 15/864,710 filed Jan. 8, 2018, issued as U.S. Pat. No. 10,745,476, which is a continuation of U.S. patent application Ser. No. 14/653,684, filed Jun. 18, 2015, issued as U.S. Pat. No. 9,879,083, which is a national stage application filed under 35 U.S.C. § 371 of International Patent Application No. PCT/US2013/076615, filed Dec. 19, 2013, which claims the benefit of and priority to U.S. Provisional Patent Application No. 61/866,787, filed Aug. 16, 2013, and U.S. Provisional Patent Application No. 61/739,435, filed Dec. 19, 2012, the entire disclosures of each application being incorporated by reference herein in their entireties.

### SEQUENCE LISTING

The instant application contains a Sequence Listing XML which has been submitted electronically in ST26 XML format and is hereby incorporated by reference in its entirety. Said ST26 XML file, created on Feb. 26, 2025, is named 406767-AVO-030USC3_216580_sequence_listing.xml and is 151,552 bytes in size.

### FIELD OF THE INVENTION

The field of the invention is molecular biology, immunology and oncology. More particularly, the field is antibodies that bind human Notch3.

### BACKGROUND

Notch pathway signaling is involved in numerous cellular processes, including cell fate determination, differentiation, proliferation, apoptosis, migration and angiogenesis. In mammals, there are four Notch proteins (sometimes called “Notch receptors”), designated Notch1-Notch4. All four Notch proteins have a similar domain structure, which includes an extracellular domain, a negative regulatory region (NRR), a single-pass transmembrane domain, and an intracellular domain. The extracellular domain contains a series of EGF-like repeats that are involved in ligand binding. During maturation, the Notch polypeptide is cleaved by a furin-like protease. This cleavage divides the Notch protein into two subunits that are held together by noncovalent interactions of the NRR. In the absence of ligand binding, the NRR domain functions to keep the Notch protein in a protease-resistant conformation. The intracellular domain is a transcription factor called Notch intracellular domain (NICD), which is released upon proteolytic cleavage by gamma secretase, in response to binding of the Notch protein by a ligand. In mammals, the Notch ligands are Delta-like (e.g., DLL1 and DLL4) and Jagged (also referred to as Jag, e.g., Jag1 and Jag2). When the NICD is released, it travels to the nucleus, where it activates transcription of the Notch-responsive genes, HES1, HES5, NRARP, Deltex1 and c-MYC. For reviews of Notch-related biology, see, e.g., Bray, 2006, NATURE REVIEWS 7:678-689; Kopan et al., 2009, CELL 137:216-233.

While Notch proteins play crucial roles in normal development, dysregulation of the Notch proteins is associated with various types of cancer, including T-cell acute lymphatic leukemia/lymphoma (T-All), breast cancer, colon cancer, ovarian cancer and lung cancer. See, e.g., Miele et al., 2006, CURRENT CANCER DRUG TARGETS 6:313-323. Accordingly, one therapeutic approach for the treatment of cancer is inhibition of Notch pathway signaling. Inhibition of Notch pathway signaling has been achieved using monoclonal antibodies (Wu et al., 2010, NATURE 464:1052-1057; Aste-Amézaga et al., 2010, PLOS ONE 5:1-13 e9094).

Naturally-occurring antibodies are multimeric proteins that contain four polypeptide chains (FIG. 1). Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (V_{L} in FIG. 1) and one constant region (C_{L} in FIG. 1). The heavy chain consists of one variable region (V_{H} in FIG. 1) and at least three constant regions (CH₁, CH₂ and CH₃ in FIG. 1). The variable regions determine the specificity of the antibody.

Each variable region contains three hypervariable regions known as complementarity determining regions (CDRs) flanked by four relatively conserved regions known as framework regions (FRs). The three CDRs, referred to as CDR₁, CDR₂, and CDR₃, contribute to the antibody binding specificity. Naturally occurring antibodies have been used as starting material for engineered antibodies, such as chimeric antibodies and humanized antibodies.

There is a need for improved antibodies that neutralize the biological activity of human Notch3 and that can be used as therapeutic agents to treat human patients.

### SUMMARY OF THE INVENTION

The invention is based on the discovery of antibodies that specifically bind human Notch3. Antibodies disclosed herein contain Notch3 binding sites based on the CDRs of the anti-Notch3 antibodies described herein. The disclosed antibodies prevent or inhibit activation of human Notch3. They do so by inhibiting Notch3 from binding to Notch ligands, i.e., Jag1, Jag2, DLL1, and DLL4. The disclosed antibodies can be used to inhibit the proliferation of tumor cells in vitro and/or in vivo. When administered to a human cancer patient, the antibodies inhibit or reduce tumor growth in the human patient.

These and other aspects and advantages of the invention are illustrated by the following figures, detailed description and claims. As used herein, “including” means without limitation, and examples cited are non-limiting. As used herein, “antibody 4F11” means antibody 4F11, or humanized variants thereof.

### DESCRIPTION OF THE DRAWINGS

The invention can be more completely understood with reference to the following drawings.

FIG. 1 (prior art) is a schematic representation of atypical naturally-occurring antibody.

FIGS. 2A-C provide the amino acid sequence corresponding to the extracellular domain (ECD; amino acids 40 to 1643) of human Notch3 (SEQ ID NO:95). The amino acid sequence begins in FIG. 2A and continues in FIGS. 2B-C.

FIG. 3 provides the amino acid sequence corresponding to EGF-like repeats 1-11 (amino acids 40 to 467 of the extracellular domain shown in FIGS. 2A-C) of human Notch3 (SEQ ID NO:96).

FIG. 4 is a bar chart summarizing results of an experiment to determine specificity of antibody 4F11 binding to Fc-bound recombinant human Notch3 (rhNotch3) using Octet. The results demonstrate that the binding specificity of the 4F11 antibody for rhNotch3 is much higher than for rhNotch1 or rhNotch2.

FIG. 5A is a bar chart illustrating the ability of antibody 4F11 to neutralize binding of the ligand Jag1 to human Notch3 protein. The chart shows that the antibody 4F11 and a Notch3 Specific Control, but not human immunoglobulin (hIgG), were able to block binding of recombinant human Fc-bound Jag1 (Jag1-Fc) to rhNotch3, as detected by bio-layer interferometry (BLI).

FIG. 5B is a bar chart illustrating the ability of antibody 4F11 to neutralize binding of the ligand Jag2 to human Notch3 protein. The chart shows that the antibody 4F11 and a Notch3 Specific Control, but not hIgG, were able to block binding of recombinant human Fc-bound Jag2 (Jag2-Fc) to rhNotch3, as detected by BLI.

FIG. 5C is a bar chart illustrating the ability of antibody 4F11 to neutralize binding of the ligand DLL1 to human Notch3 protein. The chart shows that the antibody 4F11 and a Notch3 Specific Control, but not hIgG, block binding of recombinant human Fc-bound DLL1 (DLL1-Fc) to rhNotch3, to a greater extent than IgG, as detected by BLI.

FIG. 5D is a bar chart illustrating the ability of antibody 4F11 to neutralize binding of the ligand DLL4 to human Notch3 protein. The chart shows that the antibody 4F11 and a Notch3 Specific Control, but not hIgG, were able to block binding of recombinant human Fc-bound DLL4 (DLL4-Fc) to rhNotch3, as detected by BLI.

FIG. 6A is a dose-response curve summarizing results from a Notch3 reporter assay showing that antibody 4F11 inhibits Notch3-dependent reporter gene expression in the presence of the Fc-bound ligand Jag2 (Jag2-Fc). The graph shows the relationship between inhibition of Jag2-Fc-stimulated reporter activity (% Inhibition) by antibody 4F11 in transduced cells, in relation to the amount of Jag2-Fc ligand. Reporter activity of cells transduced with the RBP-Jκ-dependent luciferase reporter, exposed to any activating ligand, and treated with mouse IgG (mIgG) was defined as 100% activity for each ligand. Reporter activity of transduced cells not exposed to ligand but treated with mouse IgG was defined as 0% activity.

FIG. 6B is a bar chart summarizing results of Notch3 reporter assays showing that antibody 4F11, but not mouse IgG (mIgG), inhibits Notch3-dependent reporter gene expression induced by the ligands hJag1, hJag2, hDLL1 and hDLL4. Reporter activity of cells transduced with the RBP-Jκ-dependent luciferase reporter, exposed to any activating ligand, and treated with mouse IgG (mIgG) was defined as 100% activity for each ligand. Reporter activity of transduced cells not exposed to ligand but treated with mouse IgG was defined as 0% activity.

FIG. 7 is a sequence alignment showing the amino acid sequence of the complete immunoglobulin heavy chain variable region of chimeric 4F11 variable region denoted as Ch4F11 and humanized 4F11 heavy chain variable regions denoted as Sh4F11 Hv3-23, Sh4F11 Hv3-23 A28T S31H T62S, Sh4F11 Hv3-23 S31H T62S, Sh4F11 Hv3-23 A28T S3TN T62S, and Sh4F11 Hv3-23 A28T T62S. The amino acid sequences for each heavy chain variable region are aligned against one another, and CDR₁, CDR₂, and CDR₃ sequences (Kabat definition) are identified in boxes. The unboxed sequences represent framework (FR) sequences.

FIG. 8 is a sequence alignment showing the isolated CDR₁, CDR₂, and CDR₃ sequences for each of the immunoglobulin heavy chain variable region sequences in FIG. 7.

FIG. 9 is a sequence alignment showing the amino acid sequence of the complete immunoglobulin light chain variable region of chimeric 4F11 denoted as Ch4F11 and humanized 4F11 light chain variable regions denoted as Hu4F11 Kv2D-29, Hu4F11 Kv2D-29 N28H, Hu4F11 Kv2D-29 N28Q, and Hu4F11 Kv2D-29 N28Y. The light chain variable region amino acid sequences are aligned against one another, and CDR₁, CDR₂, and CDR₃ sequences (Kabat definition) are identified in boxes. The unboxed sequences represent framework (FR) sequences.

FIG. 10 is a sequence alignment showing the isolated CDR₁, CDR₂, and CDR₃ sequences for each of the immunoglobulin light chain variable region sequences in FIG. 9.

FIG. 11 is a graph illustrating the ability of selected humanized 4F11 (i.e., Hu4F11-70, Hu4F11-72, Hu4F11-74, and Hu4F11-78) antibodies to inhibit ligand-induced Notch3 ICD cleavage in MDA-MB-468 cells plated on Jag2mFc. MDA-MB-468 cells exposed to either one of the 4F11 antibodies or hIgG were plated in wells coated with hJag2-mFc, incubated overnight, washed, and their lysates collected for detection of NICD by Western blot. The graph shows intensity of the cleaved NICD band in each sample as detected by Western blot. Blots were also probed with anti-β tubulin as a control, and bands were quantitated using ImageLab software. As a negative control, cells exposed to hIgG were plated in plates coated with mFc (Fc) rather than Jag2mFc.

FIG. 12A and FIG. 12B are graphs summarizing results of a Notch3 reporter assay showing that the humanized variants of 4F11 inhibit Notch3-dependent reporter gene expression induced by the ligand Jag2. CHO cells transfected with Jag2 cDNA expression vector were incubated for 24 hours with a mixture of HCC1143 reporter cells and 0.1 mg/ml, 1 mg/ml, or 10 mg/ml of 4F11 antibody variants (i.e., Hu4F11-32, Hu4F11-69, Hu4F11-70, Hu4F11-71, Hu4F11-72, Hu4F11-73, Hu4F11-74, Hu4F11-75, Hu4F11-76, Hu4F11-77, Hu4F11-78, Hu4F11-79, or Hu4F11-80), a negative control antibody (Neg Control Ab), or human immunoglobulin G (IgG), or were left untreated. 24 hours after exposing reporter cells to Jag2-expressing CHO cells, cells were processed using the Bright Glo (Promega, Madison, WI) reporter assay protocol and average relative light units (RLU) were detected on a GloMax Luminometer (Promega) (FIG. 12A and FIG. 12B).

FIG. 13 is a graph illustrating the ability of selected humanized 4F11 antibodies (i.e., Hu4F11-70, Hu4F11-72 and Hu4F11-78) to inhibit activation of Notch3 receptor in vivo after treatment of mice bearing HCC2429 xenograft tumors. Mice inoculated with matrigel and HCC2429 Notch3-expressing cells were allowed to develop tumors of 300-400 mm³, at which point three mice (m1, m2, and m3 in FIG. 13) each were treated with 20 mg/kg of hIgG, 4F11, or humanized Notch3 antibody. Tumors were then harvested and lysates collected, and Western blotting was performed using an antibody specific for the Notch3 C-terminus in order to detect the extent of Notch intracellular domain cleavage. Western blot band intensity of the cleaved Notch intracellular domain fragment was calculated, normalizing to β-tubulin loading control.

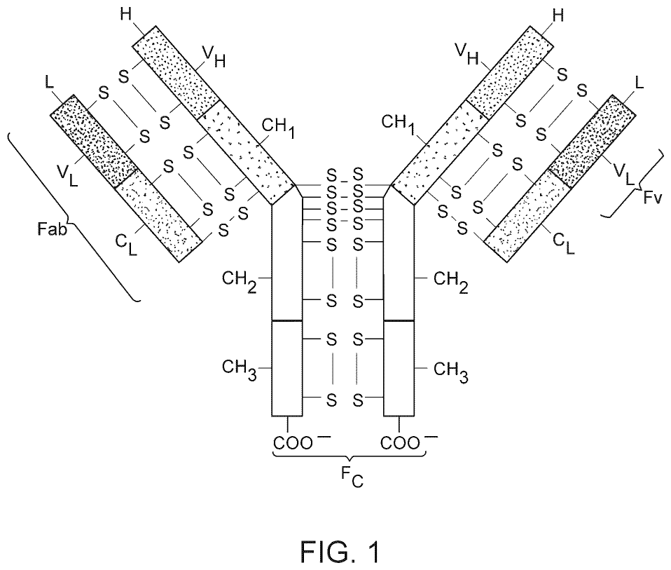

### DETAILED DESCRIPTION

The antibodies disclosed herein are based on the antigen binding sites of certain monoclonal antibodies that have been selected on the basis of binding and neutralizing the activity of human Notch3. The antibodies contain immunoglobulin variable region CDR sequences that define a binding site for human Notch3.

Because of the neutralizing activity of these antibodies, they are useful for inhibiting the growth and/or proliferation of certain cancer cells and tumors. The antibodies can be engineered (e.g., humanized) to minimize or eliminate an immune response when administered as a therapeutic antibody to a human patient. Various features and aspects of the invention are discussed in more detail below.

As used herein, unless otherwise indicated, the term “antibody” means an intact antibody (e.g., an intact monoclonal antibody) or antigen-binding fragment of an antibody, including an intact antibody or antigen-binding fragment that has been modified, engineered or chemically conjugated. Examples of antibodies that have been modified or engineered are chimeric antibodies, humanized antibodies, and multispecific antibodies (e.g., bispecific antibodies). Examples of antigen-binding fragments include Fab, Fab′, F(ab′)₂, Fv, single chain antibodies (e.g., scFv), minibodies, and diabodies. An antibody conjugated to a toxin moiety is an example of a chemically conjugated antibody.

I. Antibodies that Bind Human Notch3

As disclosed herein, the antibodies may comprise: (a) an immunoglobulin heavy chain variable region comprising the structure CDR_{H1}-CDR_{H2}-CDR_{H3} and (b) an immunoglobulin light chain variable region comprising the structure CDR_{L1}-CDR_{L2}-CDR_{L3}, wherein the heavy chain variable region and the light chain variable region together define a single binding site for binding human Notch3.

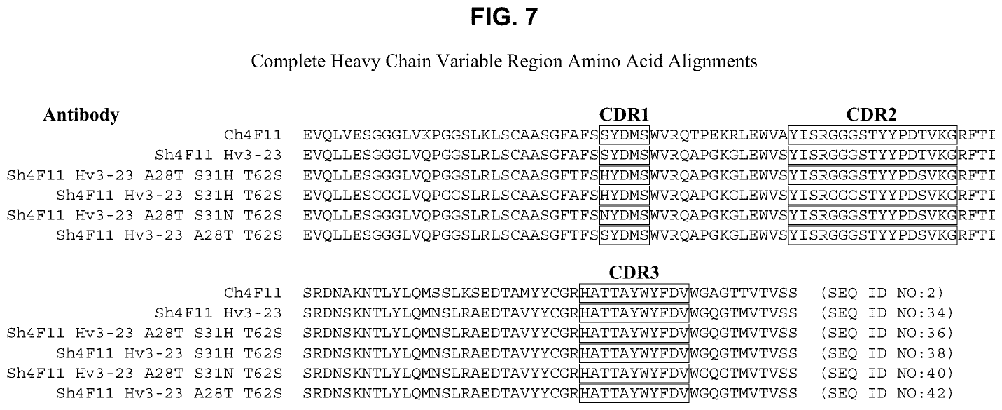

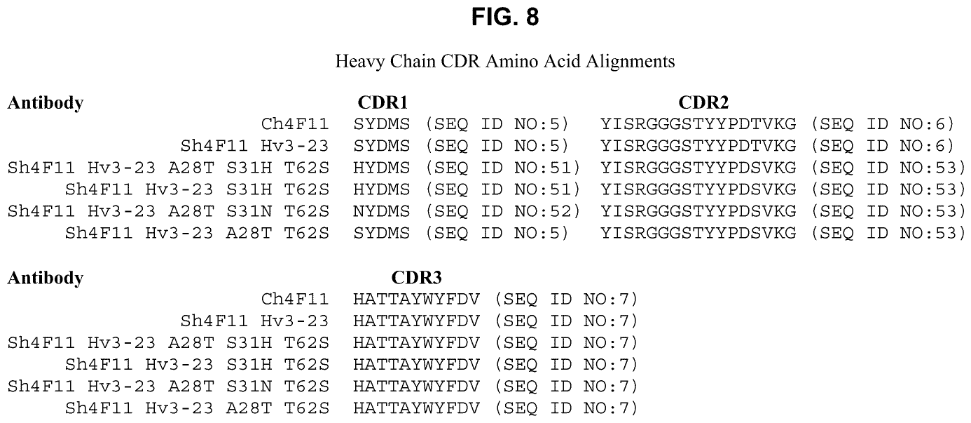

In some embodiments, the antibody comprises: (a) an immunoglobulin heavy chain variable region comprising the structure CDR_{H1}-CDR_{H2}-CDR_{H3} and (b) an immunoglobulin light chain variable region, wherein the heavy chain variable region and the light chain variable region together define a single binding site for binding human Notch3. A CDR_{H1} comprises an amino acid sequence selected from the group consisting of SEQ ID NO:5, SEQ ID NO:11, SEQ ID NO:14, SEQ ID NO:51, SEQ ID NO:52, SEQ ID NO:54, SEQ ID NO:55, SEQ ID NO:56, SEQ ID NO:57, SEQ ID NO:58, SEQ ID NO:59, SEQ ID NO:60 and SEQ ID NO:61; a CDR_{H2} comprises an amino acid sequence selected from the group consisting of SEQ ID NO:6, SEQ ID NO:12, SEQ ID NO:15 and SEQ ID NO:53; and a CDR_{H3} comprises an amino acid sequence selected from the group consisting of SEQ ID NO:7 and SEQ ID NO:16. (The immunoglobulin heavy chain variable regions including the referenced CDR sequences can be found in Table 10 and FIGS. 7-8.)

In some embodiments, the antibody comprises an immunoglobulin heavy chain variable region comprising a CDR_{H1} comprising an amino acid sequence selected from the group consisting of SEQ ID NO: 5 and SEQ ID NO: 11, a CDR_{H2} comprising the amino acid sequence of SEQ ID NO: 6, and a CDR_{H3} comprising the amino acid sequence of SEQ ID NO: 7.

In some embodiments, the antibody comprises an immunoglobulin heavy chain variable region comprising a CDR_{H1} comprising an amino acid sequence selected from the group consisting of SEQ ID NO: 51 and SEQ ID NO: 54, a CDR_{H2} comprising the amino acid sequence of SEQ ID NO: 53, and a CDR_{H3} comprising the amino acid sequence of SEQ ID NO: 7.

In some embodiments, the antibody comprises an immunoglobulin heavy chain variable region comprising a CDR_{H1} comprising an amino acid sequence selected from the group consisting of SEQ ID NO: 51 and SEQ ID NO: 55, a CDR_{H2} comprising the amino acid sequence of SEQ ID NO: 53, and a CDR_{H3} comprising the amino acid sequence of SEQ ID NO: 7.

In some embodiments, the antibody comprises an immunoglobulin heavy chain variable region comprising a CDR_{H1} comprising an amino acid sequence selected from the group consisting of SEQ ID NO: 52 and SEQ ID NO: 56, a CDR_{H2} comprising the amino acid sequence of SEQ ID NO: 53, and a CDR_{H3} comprising the amino acid sequence of SEQ ID NO: 7.

In some embodiments, the antibody comprises an immunoglobulin heavy chain variable region comprising a CDR_{H1} comprising an amino acid sequence selected from the group consisting of SEQ ID NO: 5 and SEQ ID NO: 57, a CDR_{H2} comprising the amino acid sequence of SEQ ID NO: 53, and a CDR_{H3} comprising the amino acid sequence of SEQ ID NO: 7.

Preferably, the CDR_{H1}, CDR_{H2}, and CDR_{H3} sequences are interposed between human or humanized immunoglobulin FR sequences.

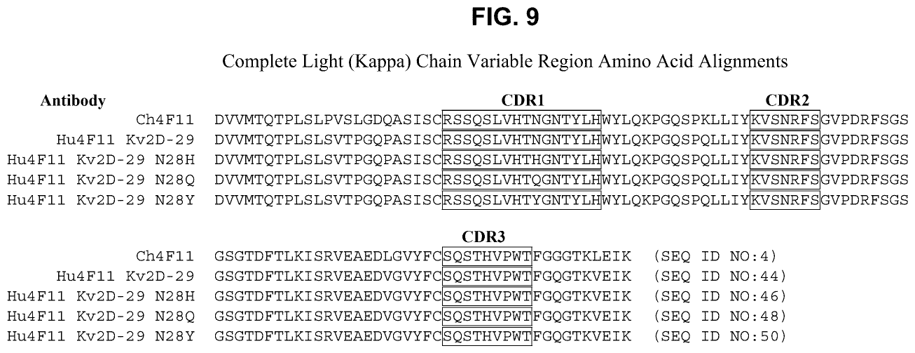

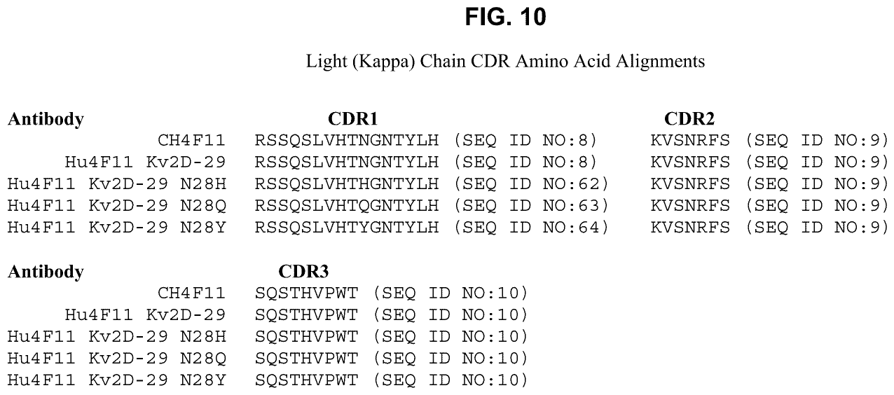

In other embodiments, the antibody comprises (a) an immunoglobulin light chain variable region comprising the structure CDR_{L1}-CDR_{L2}-CDR_{L3}, and (b) an immunoglobulin heavy chain variable region, wherein the IgG light chain variable region and the IgG heavy chain variable region together define a single binding site for binding human Notch3. A CDR_{L1} comprises an amino acid sequence selected from the group consisting of SEQ ID NO:8, SEQ ID NO:20, SEQ ID NO:62, SEQ ID NO:63, SEQ ID NO:64, SEQ ID NO:65, SEQ ID NO:66 and SEQ ID NO:67; a CDR_{L2} comprises an amino acid sequence selected from the group consisting of SEQ ID NO:9 or the amino acid sequence of KVS; and a CDR_{L3} comprises an amino acid sequence of SEQ ID NO:10. (The immunoglobulin light chain variable regions including the referenced CDR sequences can be found in Table 11 and FIGS. 9-10.)

In some embodiments, the antibody comprises an immunoglobulin light chain variable region comprising a CDR_{L1} comprising the amino acid sequence of SEQ ID NO: 8, a CDR_{L2} comprising the amino acid sequence of SEQ ID NO: 9, and a CDR_{L3} comprising the amino acid sequence of SEQ ID NO: 10.

In some embodiments, the antibody comprises an immunoglobulin light chain variable region comprising a CDR_{L1} comprising the amino acid sequence of SEQ ID NO: 62, a CDR_{L2} comprising the amino acid sequence of SEQ ID NO: 9, and a CDR_{L3} comprising the amino acid sequence of SEQ ID NO: 10.

In some embodiments, the antibody comprises an immunoglobulin light chain variable region comprising a CDR_{L1} comprising the amino acid sequence of SEQ ID NO: 63, a CDR_{L2} comprising the amino acid sequence of SEQ ID NO: 9, and a CDR_{L3} comprising the amino acid sequence of SEQ ID NO: 10.

In some embodiments, the antibody comprises an immunoglobulin light chain variable region comprising a CDR_{L1} comprising the amino acid sequence of SEQ ID NO: 64, a CDR_{L2} comprising the amino acid sequence of SEQ ID NO: 9, and a CDR_{L3} comprising the amino acid sequence of SEQ ID NO: 10.

Preferably, the CDR_{L1}, CDR_{L2}, and CDR_{L3} sequences are interposed between human or humanized immunoglobulin FR sequences.

In some embodiments, the antibody comprises: (a) an immunoglobulin heavy chain variable region comprising the structure CDR_{H1}-CDR_{H2}-CDR_{H3} and (b) an immunoglobulin light chain variable region comprising the structure CDR_{L1}-CDR_{L2}-CDR_{L3}, wherein the heavy chain variable region and the light chain variable region together define a single binding site for binding human Notch3. The CDR_{H1} comprises an amino acid sequence selected from the group consisting of SEQ ID NO:5, SEQ ID NO:11, SEQ ID NO:14, SEQ ID NO:51, SEQ ID NO:52, SEQ ID NO:54, SEQ ID NO:55, SEQ ID NO:56, SEQ ID NO:57, SEQ ID NO:58, SEQ ID NO:59, SEQ ID NO:60 and SEQ ID NO:61; the CDR_{H2} comprises an amino acid sequence selected from the group consisting of SEQ ID NO:6, SEQ ID NO:12, SEQ ID NO: 15 and SEQ ID NO:53; and the CDR_{H3} comprises an amino acid sequence selected from the group consisting of SEQ ID NO:7 and SEQ ID NO:16. The CDR_{L1} comprises an amino acid sequence selected from the group consisting of SEQ ID NO:8, SEQ ID NO:20, SEQ ID NO:62, SEQ ID NO:63, SEQ ID NO:64, SEQ ID NO:65, SEQ ID NO:66 and SEQ ID NO:67; the CDR_{L2} comprises an amino acid sequence selected from the group consisting of SEQ ID NO:9 or the amino acid sequence of KVS; and the CDR_{L3} comprises an amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:5 and SEQ ID NO:11, a CDR_{H2} comprising the amino acid sequence of SEQ ID NO:6, and a CDR_{H3} comprising the amino acid sequence of SEQ ID NO:7; and an immunoglobulin light chain variable region comprising a CDR_{L1} comprising the amino acid sequence of SEQ ID NO:8, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO: 5 and SEQ ID NO:11; a CDR_{H2} comprising the amino acid sequence of SEQ ID NO: 6; and a CDR_{H3} comprising the amino acid sequence of SEQ ID NO: 7; and an immunoglobulin light chain variable region comprising a CDR_{L1} comprising the amino acid sequence of SEQ ID NO: 62; a CDR_{L2} comprising the amino acid sequence of SEQ ID NO: 9; and a CDR_{L3} comprising the amino acid sequence of SEQ ID NO: 10.

In some embodiments, the antibody comprises an immunoglobulin heavy chain variable region comprising a CDR_{H1} comprising an amino acid sequence selected from the group consisting of SEQ ID NO: 5 and SEQ ID NO:11; a CDR_{H2} comprising the amino acid sequence of SEQ ID NO: 6; and a CDR_{H3} comprising the amino acid sequence of SEQ ID NO: 7; and an immunoglobulin light chain variable region comprising a CDR_{L1} comprising the amino acid sequence of SEQ ID NO: 63; a CDR_{L2} comprising the amino acid sequence of SEQ ID NO: 9; and a CDR_{L3} comprising the amino acid sequence of SEQ ID NO: 10.

In some embodiments, the antibody comprises an immunoglobulin heavy chain variable region comprising a CDR_{H1} comprising an amino acid sequence selected from the group consisting of SEQ ID NO: 5 and SEQ ID NO:11; a CDR_{H2} comprising the amino acid sequence of SEQ ID NO: 6; and a CDR_{H3} comprising the amino acid sequence of SEQ ID NO: 7; and an immunoglobulin light chain variable region comprising a CDR_{L1} comprising the amino acid sequence of SEQ ID NO: 64; a CDR_{L2} comprising the amino acid sequence of SEQ ID NO: 9; and a CDR_{L3} comprising the amino acid sequence of SEQ ID NO: 10.

In some embodiments, the antibody comprises an immunoglobulin heavy chain variable region comprising a CDR_{H1} comprising an amino acid sequence selected from the group consisting of SEQ ID NO:51 and SEQ ID NO: 54, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:8, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:51 and SEQ ID NO: 54, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:62, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:51 and SEQ ID NO: 54, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:63, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:51 and SEQ ID NO: 54, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:64, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:51 and SEQ ID NO: 55, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:8, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:51 and SEQ ID NO: 55, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:62, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:51 and SEQ ID NO: 55, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:63, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:51 and SEQ ID NO: 55, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:64, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:52 and SEQ ID NO: 56, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:8, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:52 and SEQ ID NO: 56, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:62, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:52 and SEQ ID NO: 56, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:63, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:52 and SEQ ID NO: 56, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:64, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:5 and SEQ ID NO: 57, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:8, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:5 and SEQ ID NO: 57, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:62, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:5 and SEQ ID NO: 57, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:63, a CDR_{L2} comprising the amino acid sequence of SEQ ID NO:9, and a CDR_{L3} comprising the amino acid sequence of SEQ ID NO:10.

In some embodiments, the antibody comprises an immunoglobulin heavy chain variable region comprising a CDR_{H1} comprising an amino acid sequence selected from the group consisting of SEQ ID NO:5 and SEQ ID NO: 57, a CDR_{H2} comprising the amino acid sequence of SEQ ID NO:53, and a CDR_{H3} comprising the amino acid sequence of SEQ ID NO:7; and an immunoglobulin light chain variable region comprising a CDR_{L1} comprising the amino acid sequence of SEQ ID NO:64, a CDR_{L2} comprising the amino acid sequence of SEQ ID NO:9, and a CDR_{L3} comprising the amino acid sequence of SEQ ID NO:10.

The antibodies disclosed herein comprise an immunoglobulin heavy chain variable region and an immunoglobulin light chain variable region. In some embodiments, the antibody comprises an immunoglobulin heavy chain variable region selected from the group consisting of SEQ ID NO:2 (4F11, Ch4F11 Chimeric); SEQ ID NO:34 (Sh4F11 Hv3-23); SEQ ID NO:36 (Sh4F11 Hv3-23 A28T S31H T62S); SEQ ID NO:38 (Sh4F11 Hv3-23 S31H T62S); SEQ ID NO:40 (Sh4F11 Hv3-23 A28T S31N T62S); and SEQ ID NO:42 (Sh4F11 Hv3-23 A28T T62S); and an immunoglobulin light chain variable region. As a convenience for the reader, certain SEQ ID NOs. are followed by a parenthetical including the antibody designation that was the origin of the sequence. For example, “SEQ ID NO:2 (4F11, Ch4F11 Chimeric)” means that SEQ ID NO:2 comes from murine antibody 4F11. SEQ ID NO:2 is also found in the chimeric 4F11 sequence.

In other embodiments, the antibody comprises an immunoglobulin light chain variable region selected from the group consisting of SEQ ID NO:4 (4F11, Ch4F11 Chimeric); SEQ ID NO:44 (Hu4F11 Kv2D-29); SEQ ID NO:46 (Hu4F11 Kv2D-29 N28H); SEQ ID NO:48 (Hu4F11 Kv2D-29 N28Q); and SEQ ID NO:50 (Hu4F11 Kv2D-29 N28Y); and an immunoglobulin heavy chain variable region.

In some embodiments, the antibody comprises an immunoglobulin heavy chain variable region selected from the group consisting of SEQ ID NO:2 (4F11, Ch4F11 Chimeric); SEQ ID NO:34 (Sh4F11 Hv3-23); SEQ ID NO:36 (Sh4F11 Hv3-23 A28T S31H T62S); SEQ ID NO:38 (Sh4F11 Hv3-23 S31H T62S); SEQ ID NO:40 (Sh4F11 Hv3-23 A28T S31N T62S); and SEQ ID NO:42 (Sh4F11 Hv3-23 A28T T62S), and an immunoglobulin light chain variable region selected from the group consisting of SEQ ID NO:4 (4F11, Ch4F11 Chimeric); SEQ ID NO:44 (Hu4F11 Kv2D-29); SEQ ID NO:46 (Hu4F11 Kv2D-29 N28H); SEQ ID NO:48 (Hu4F11 Kv2D-29 N28Q); and SEQ ID NO:50 (Hu4F11 Kv2D-29 N28Y).

In some embodiments, the antibody comprises an immunoglobulin heavy chain variable region comprising the amino acid sequence of SEQ ID NO:2 (4F11, Ch4F11 Chimeric), and an immunoglobulin light chain variable region comprising the amino acid sequence of SEQ ID NO:4 (4F11, Ch4F11 Chimeric).

In some embodiments, the antibody comprises an immunoglobulin heavy chain variable region comprising the amino acid sequence of SEQ ID NO:2 (4F11, Ch4F11 Chimeric), and an immunoglobulin light chain variable region comprising the amino acid sequence of SEQ ID NO:44 (Hu4F11 Kv2D-29).

In some embodiments, the antibody comprises an immunoglobulin heavy chain variable region comprising the amino acid sequence of SEQ ID NO:34 (Sh4F11 Hv3-23), and an immunoglobulin light chain variable region comprising the amino acid sequence of SEQ ID NO:4 (4F11, Ch4F11 Chimeric).

In some embodiments, the antibody comprises an immunoglobulin heavy chain variable region comprising the amino acid sequence of SEQ ID NO:34 (Sh4F11 Hv3-23), and an immunoglobulin light chain variable region comprising the amino acid sequence of SEQ ID NO:44 (Hu4F11 Kv2D-29).

In some embodiments, the antibody comprises an immunoglobulin heavy chain variable region comprising the amino acid sequence of SEQ ID NO:36 (Sh4F11 Hv3-23 A28T S31H T62S), and an immunoglobulin light chain variable region comprising the amino acid sequence of SEQ ID NO:46 (Hu4F11 Kv2D-29 N28H).

In some embodiments, the antibody comprises an immunoglobulin heavy chain variable region comprising the amino acid sequence of SEQ ID NO:38 (Sh4F11 Hv3-23 S31H T62S), and an immunoglobulin light chain variable region comprising the amino acid sequence of SEQ ID NO:46 (Hu4F11 Kv2D-29 N28H).

In some embodiments, the antibody comprises an immunoglobulin heavy chain variable region comprising the amino acid sequence of SEQ ID NO:40 (Sh4F11 Hv3-23 A28T S31N T62S), and an immunoglobulin light chain variable region comprising the amino acid sequence of SEQ ID NO:46 (Hu4F11 Kv2D-29 N28H).

In some embodiments, the antibody comprises an immunoglobulin heavy chain variable region comprising the amino acid sequence of SEQ ID NO:42 (Sh4F11 Hv3-23 A28T T62S), and an immunoglobulin light chain variable region comprising the amino acid sequence of SEQ ID NO:46 (Hu4F11 Kv2D-29 N28H).

In some embodiments, the antibody comprises an immunoglobulin heavy chain variable region comprising the amino acid sequence of SEQ ID NO:36 (Sh4F11 Hv3-23 A28T S31H T62S), and an immunoglobulin light chain variable region comprising the amino acid sequence of SEQ ID NO:48 (Hu4F11 Kv2D-29 N28Q).

In some embodiments, the antibody comprises an immunoglobulin heavy chain variable region comprising the amino acid sequence of SEQ ID NO:38 (Sh4F11 Hv3-23 S31H T62S), and an immunoglobulin light chain variable region comprising the amino acid sequence of SEQ ID NO:48 (Hu4F11 Kv2D-29 N28Q).

In some embodiments, the antibody comprises an immunoglobulin heavy chain variable region comprising the amino acid sequence of SEQ ID NO:40 (Sh4F11 Hv3-23 A28T S31N T62S), and an immunoglobulin light chain variable region comprising the amino acid sequence of SEQ ID NO:48 (Hu4F11 Kv2D-29 N28Q).

In some embodiments, the antibody comprises an immunoglobulin heavy chain variable region comprising the amino acid sequence of SEQ ID NO:42 (Sh4F11 Hv3-23 A28T T62S), and an immunoglobulin light chain variable region comprising the amino acid sequence of SEQ ID NO:48 (Hu4F11 Kv2D-29 N28Q).

In some embodiments, the antibody comprises an immunoglobulin heavy chain variable region comprising the amino acid sequence of SEQ ID NO:36 (Sh4F11 Hv3-23 A28T S31H T62S), and an immunoglobulin light chain variable region comprising the amino acid sequence of SEQ ID NO:50 (Hu4F11 Kv2D-29 N28Y).

In some embodiments, the antibody comprises an immunoglobulin heavy chain variable region comprising the amino acid sequence of SEQ ID NO:38 (Sh4F11 Hv3-23 S31H T62S), and an immunoglobulin light chain variable region comprising the amino acid sequence of SEQ ID NO:50 (Hu4F11 Kv2D-29 N28Y).

In some embodiments, the antibody comprises an immunoglobulin heavy chain variable region comprising the amino acid sequence of SEQ ID NO:40 (Sh4F11 Hv3-23 A28T S31N T62S), and an immunoglobulin light chain variable region comprising the amino acid sequence of SEQ ID NO:50 (Hu4F11 Kv2D-29 N28Y).

In some embodiments, the antibody comprises an immunoglobulin heavy chain variable region comprising the amino acid sequence of SEQ ID NO:42 (Sh4F11 Hv3-23 A28T T62S), and an immunoglobulin light chain variable region comprising the amino acid sequence of SEQ ID NO:50 (Hu4F11 Kv2D-29 N28Y).

In certain embodiments, the antibodies disclosed herein comprise an immunoglobulin heavy chain and an immunoglobulin light chain. In some embodiments, the antibody comprises an immunoglobulin heavy chain selected from the group consisting of SEQ ID NO: 27 (4F11); SEQ ID NO:74 (Ch4F11 Chimeric); SEQ ID NO:76 (Sh4F11 Hv3-23); SEQ ID NO:78 (Sh4F11 Hv3-23 A28T S31H T62S); SEQ ID NO:80 (Sh4F11 Hv3-23 S31H T62S); SEQ ID NO:82 (Sh4F11 Hv3-23 A28T S31N T62S); and SEQ ID NO:84 (Sh4F11 Hv3-23 A28T T62S); and an immunoglobulin light chain.

In other embodiments, the antibody comprises an immunoglobulin light chain selected from the group consisting of SEQ ID NO:29 (4F11); SEQ ID NO:86 (Ch4F11 Chimeric); SEQ ID NO:88 (Hu4F11 Kv2D-29 Kappa); SEQ ID NO:90 (Hu4F11 Kv2D-29 N28H Kappa); SEQ ID NO:92 (Hu4F11 Kv2D-29 N28Q Kappa); and SEQ ID NO:94 (Hu4F11 Kv2D-29 N28Y Kappa); and an immunoglobulin heavy chain.

In some embodiments, the antibody comprises an immunoglobulin heavy chain selected from the group consisting of SEQ ID NO: 27 (4F11); SEQ ID NO:74 (Ch4F11 Chimeric); SEQ ID NO:76 (Sh4F11 Hv3-23); SEQ ID NO:78 (Sh4F11 Hv3-23 A28T S31H T62S); SEQ ID NO:80 (Sh4F11 Hv3-23 S31H T62S); SEQ ID NO:82 (Sh4F11 Hv3-23 A28T S31N T62S); and SEQ ID NO:84 (Sh4F11 Hv3-23 A28T T62S); and an immunoglobulin light chain selected from the group consisting of SEQ ID NO:29 (4F11); SEQ ID NO:86 (Ch4F11 Chimeric); SEQ ID NO:88 (Hu4F11 Kv2D-29 Kappa); SEQ ID NO:90 (Hu4F11 Kv2D-29 N28H Kappa); SEQ ID NO:92 (Hu4F11 Kv2D-29 N28Q Kappa); and SEQ ID NO:94 (Hu4F11 Kv2D-29 N28Y Kappa).

In some embodiments, the antibody comprises an immunoglobulin heavy chain comprising the amino acid sequence of SEQ ID NO: 27 (4F11), and an immunoglobulin light chain comprising the amino acid sequence of SEQ ID NO: 29 (4F11).

In some embodiments, the antibody comprises an immunoglobulin heavy chain comprising the amino acid sequence of SEQ ID NO: 74 (Ch4F11), and an immunoglobulin light chain comprising the amino acid sequence of SEQ ID NO: 86 (Ch4F11).

In some embodiments, the antibody comprises an immunoglobulin heavy chain comprising the amino acid sequence of SEQ ID NO: 74 (Ch4F11), and an immunoglobulin light chain comprising the amino acid sequence of SEQ ID NO: 88 (Hu4F11 Kv2D-29).

In some embodiments, the antibody comprises an immunoglobulin heavy chain comprising the amino acid sequence of SEQ ID NO: 76 (Sh4F11 Hv3-23), and an immunoglobulin light chain comprising the amino acid sequence of SEQ ID NO: 86 (Ch4F11).

In some embodiments, the antibody comprises an immunoglobulin heavy chain comprising the amino acid sequence of SEQ ID NO: 76 (Sh4F11 Hv3-23), and an immunoglobulin light chain comprising the amino acid sequence of SEQ ID NO: 88 (Hu4F11 Kv2D-29).

In some embodiments, the antibody comprises an immunoglobulin heavy chain comprising the amino acid sequence of SEQ ID NO: 78 (Sh4F11 Hv3-23 A28T S31H T62S), and an immunoglobulin light chain comprising the amino acid sequence of SEQ ID NO: 90 (Hu4F11 Kv2D-29 N28H Kappa).

In some embodiments, the antibody comprises an immunoglobulin heavy chain comprising the amino acid sequence of SEQ ID NO: 80 (Sh4F11 Hv3-23 S31H T62S), and an immunoglobulin light chain comprising the amino acid sequence of SEQ ID NO: 90 (Hu4F11 Kv2D-29 N28H Kappa).

In some embodiments, the antibody comprises an immunoglobulin heavy chain comprising the amino acid sequence of SEQ ID NO: 82 (Sh4F11 Hv3-23 A28T S31N T62S), and an immunoglobulin light chain comprising the amino acid sequence of SEQ ID NO: 90 (Hu4F11 Kv2D-29 N28H Kappa).

In some embodiments, the antibody comprises an immunoglobulin heavy chain comprising the amino acid sequence of SEQ ID NO: 84 (Sh4F11 Hv3-23 A28T T62S), and an immunoglobulin light chain comprising the amino acid sequence of SEQ ID NO: 90 (Hu4F11 Kv2D-29 N28H Kappa).

In some embodiments, the antibody comprises an immunoglobulin heavy chain comprising the amino acid sequence of SEQ ID NO: 78 (Sh4F11 Hv3-23 A28T S31H T62S), and an immunoglobulin light chain comprising the amino acid sequence of SEQ ID NO: 92 (Hu4F11 Kv2D-29 N28Q Kappa).

In some embodiments, the antibody comprises an immunoglobulin heavy chain comprising the amino acid sequence of SEQ ID NO: 80 (Sh4F11 Hv3-23 S31H T62S), and an immunoglobulin light chain comprising the amino acid sequence of SEQ ID NO: 92 (Hu4F11 Kv2D-29 N28Q Kappa).

In some embodiments, the antibody comprises an immunoglobulin heavy chain comprising the amino acid sequence of SEQ ID NO: 82 (Sh4F11 Hv3-23 A28T S31N T62S), and an immunoglobulin light chain comprising the amino acid sequence of SEQ ID NO: 92 (Hu4F11 Kv2D-29 N28Q Kappa).

In some embodiments, the antibody comprises an immunoglobulin heavy chain comprising the amino acid sequence of SEQ ID NO: 84 (Sh4F11 Hv3-23 A28T T62S), and an immunoglobulin light chain comprising the amino acid sequence of SEQ ID NO: 92 (Hu4F11 Kv2D-29 N28Q Kappa).

In some embodiments, the antibody comprises an immunoglobulin heavy chain comprising the amino acid sequence of SEQ ID NO: 78 (Sh4F11 Hv3-23 A28T S31H T62S), and an immunoglobulin light chain comprising the amino acid sequence of SEQ ID NO: 94 (Hu4F11 Kv2D-29 N28Y Kappa).

In some embodiments, the antibody comprises an immunoglobulin heavy chain comprising the amino acid sequence of SEQ ID NO: 80 (Sh4F11 Hv3-23 S31H T62S), and an immunoglobulin light chain comprising the amino acid sequence of SEQ ID NO: 94 (Hu4F11 Kv2D-29 N28Y Kappa).

In some embodiments, the antibody comprises an immunoglobulin heavy chain comprising the amino acid sequence of SEQ ID NO: 82 (Sh4F11 Hv3-23 A28T S31N T62S), and an immunoglobulin light chain comprising the amino acid sequence of SEQ ID NO: 94 (Hu4F11 Kv2D-29 N28Y Kappa).

In some embodiments, the antibody comprises an immunoglobulin heavy chain comprising the amino acid sequence of SEQ ID NO: 84 (Sh4F11 Hv3-23 A28T T62S), and an immunoglobulin light chain comprising the amino acid sequence of SEQ ID NO: 94 (Hu4F11 Kv2D-29 N28Y Kappa).

In other embodiments, an isolated antibody that binds human Notch3 comprises an immunoglobulin heavy chain variable region comprising an amino acid sequence that is at least 70%, 75%, 80%, 85%, 90%, 95%, 98%, or 99% identical to the entire variable region or the FR sequence of SEQ ID NO:2 (4F11, Ch4F11 Chimeric); SEQ ID NO:34 (Sh4F11 Hv3-23); SEQ ID NO:36 (Sh4F11 Hv3-23 A28T S31H T62S); SEQ ID NO:38 (Sh4F11 Hv3-23 S31H T62S); SEQ ID NO:40 (Sh4F11 Hv3-23 A28T S31N T62S); or SEQ ID NO:42 (Sh4F11 Hv3-23 A28T T62S).

In other embodiments, an isolated antibody that binds human Notch3 comprises an immunoglobulin light chain variable region comprising an amino acid sequence that is at least 70%, 75%, 80%, 85%, 90%, 95%, 98%, or 99% identical to the entire variable region or the FR sequence of SEQ ID NO:4 (4F11, Ch4F11 Chimeric); SEQ ID NO:44 (Hu4F11 Kv2D-29); SEQ ID NO:46 (Hu4F11 Kv2D-29 N28H); SEQ ID NO:48 (Hu4F11 Kv2D-29 N28Q); or SEQ ID NO:50 (Hu4F11 Kv2D-29 N28Y).

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, herein incorporated by reference) are tailored for sequence similarity searching. For a discussion of basic issues in searching sequence databases see Altschul et al., (1994) NATURE GENETICS 6:119-129, herein fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: —G, Cost to open gap [Integer]: default=5 for nucleotides/11 for proteins; —E, Cost to extend gap [Integer]: default=2 for nucleotides/1 for proteins; —q, Penalty for nucleotide mismatch [Integer]: default=−3; —r, reward for nucleotide match [Integer]: default=1; —e, expect value [Real]: default=10; —W, wordsize [Integer]: default=11 for nucleotides/28 for megablast/3 for proteins; —y, Dropoff (X) for blast extensions in bits: default=20 for blastn/7 for others; —X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In each of the foregoing embodiments, it is contemplated herein that immunoglobulin heavy chain variable region sequences and/or light chain variable region sequences that together bind human Notch3 may contain amino acid alterations (e.g., at least 1, 2, 3, 4, 5, or 10 amino acid substitutions, deletions, or additions) in the framework regions of the heavy and/or light chain variable regions.

In certain embodiments, an isolated antibody binds human Notch3 with a K_{D} of about 35 nM, 25 nM, 15 nM 10 nM, 5 nM, 1 nM, 900 pM, 850 pM, 800 pM, 750 pM, 700 pM, 650 pM, 600 pM, 500 pM, 400 pM, 300 pM, 250 pM, 200 pM or less. Unless otherwise specified, K_{D} values are determined by surface plasmon resonance methods under the conditions described in Examples 3 and 9.

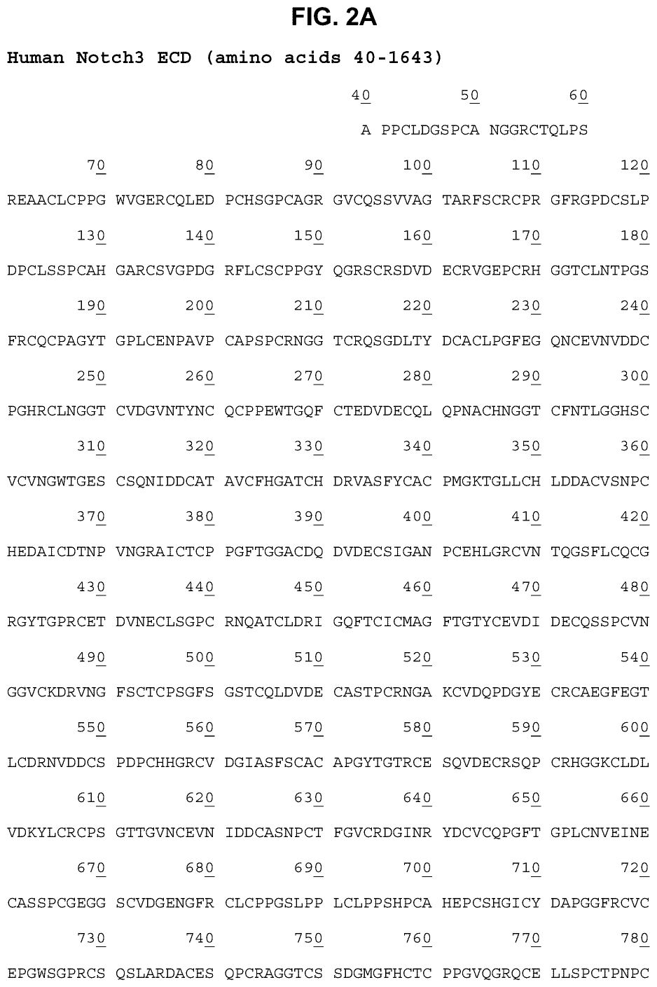

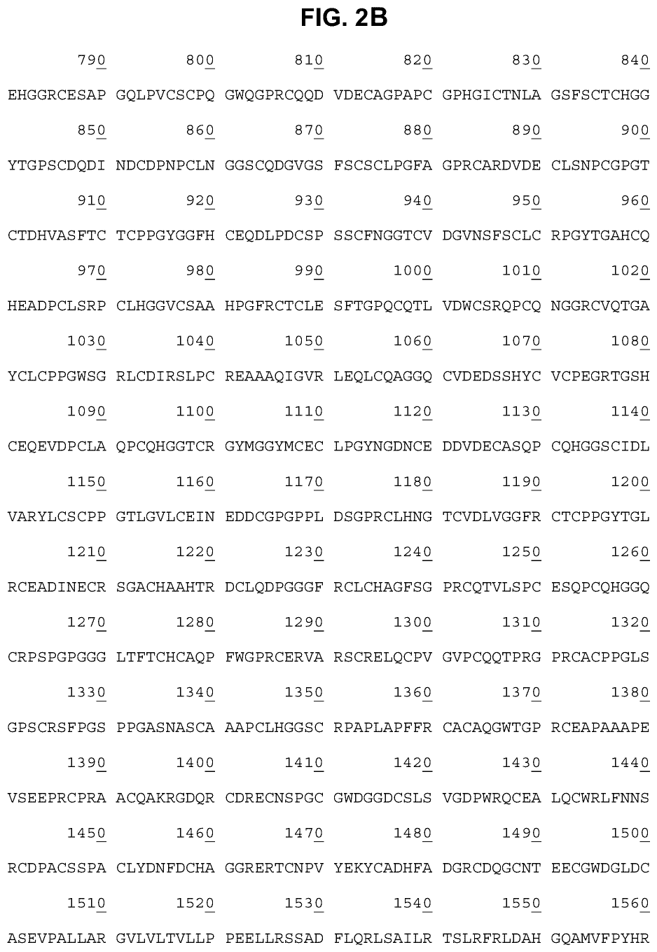

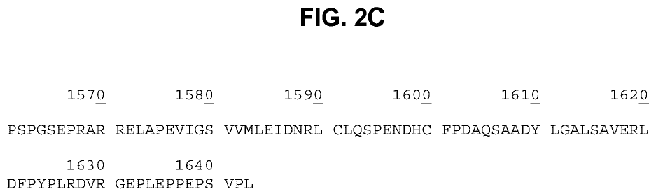

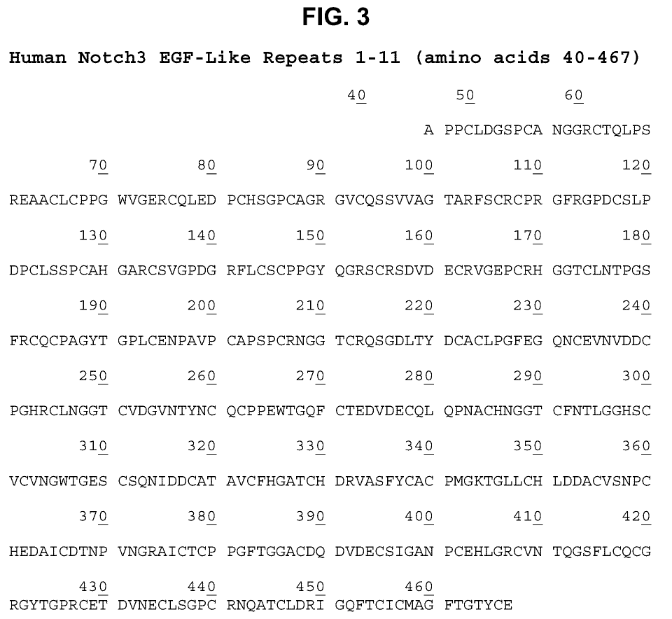

In some embodiments, monoclonal antibodies bind to the same epitope on human Notch3 as antibody 4F11. In some embodiments, monoclonal antibodies compete for binding to human Notch3 with antibody 4F11. For example, monoclonal antibodies may compete for binding to the extracellular domain (ECD) of Notch3 with antibody 4F11 (amino acid sequence corresponding to the human Notch3 ECD is shown in FIGS. 2A-C). In another example, monoclonal antibodies may compete for binding to EGF-like repeats 1-11 of human Notch3 with antibody 4F11 (amino acid sequence corresponding to EGF-like repeats 1-11 of human Notch3 is shown in FIG. 3).

Competition assays for determining whether an antibody binds to the same epitope as, or competes for binding with, antibody 4F11 are known in the art. Exemplary competition assays include immunoassays (e.g., ELISA assays, RIA assays), BIAcore analysis, biolayer interferometry and flow cytometry.

Typically, a competition assay involves the use of an antigen (e.g., a human Notch3 protein or fragment thereof) bound to a solid surface or expressed on a cell surface, a test anti-Notch3-binding antibody and a reference antibody (i.e., antibody 4F11). The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (i.e., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

In an exemplary competition assay, a reference anti-Notch3 antibody (i.e., antibody 4F11) is biotinylated using commercially available reagents. The biotinylated reference antibody is mixed with serial dilutions of the test antibody or unlabeled reference antibody (self-competition control) resulting in a mixture of various molar ratios (e.g., 1×, 5×, 10×, 20× or 100×) of test antibody (or unlabeled reference antibody) to labeled reference antibody. The antibody mixture is added to a human Notch3 (e.g., extracellular domain of human Notch3) polypeptide coated-ELISA plate. The plate is then washed and horseradish peroxidase (HRP)-strepavidin is added to the plate as the detection reagent. The amount of labeled reference antibody bound to the target antigen is detected following addition of a chromogenic substrate (e.g., TMB (3,3′,5,5′-tetramethylbenzidine) or ABTS (2,2″-azino-di-(3-ethylbenzthiazoline-6-sulfonate)), which are known in the art. Optical density readings (OD units) are measured using a SpectraMax® M2 spectrometer (Molecular Devices). OD units corresponding to zero percent inhibition are determined from wells without any competing antibody. OD units corresponding to 100% inhibition, i.e., the assay background are determined from wells without any labeled reference antibody or test antibody. Percent inhibition of labeled reference antibody to Notch3 by the test antibody (or the unlabeled reference antibody) at each concentration is calculated as follows: % inhibition=(1−(OD units−100% inhibition)/(0% inhibition−100% inhibition))*100. Persons skilled in the art will appreciate that the competition assay can be performed using various detection systems known in the art.

A competition assay may be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of antibody reduce or eliminate binding of the other.

### II. Antibody Production

Methods for producing antibodies of the invention are known in the art. For example, DNA molecules encoding light chain variable regions and/or heavy chain variable regions can be chemically synthesized using the sequence information provided herein. Synthetic DNA molecules can be ligated to other appropriate nucleotide sequences, including, e.g., constant region coding sequences, and expression control sequences, to produce conventional gene expression constructs encoding the desired antibody. Production of defined gene constructs is within routine skill in the art. Alternatively, the sequences provided herein can be cloned out of hybridomas by conventional hybridization techniques or polymerase chain reaction (PCR) techniques, using synthetic nucleic acid probes whose sequences are based on sequence information provided herein, or prior art sequence information regarding genes encoding the heavy and light chains of murine antibodies in hybridoma cells.

Nucleic acids encoding desired antibodies can be incorporated (ligated) into expression vectors, which can be introduced into host cells through conventional transfection or transformation techniques. Exemplary host cells are E. coli cells, Chinese hamster ovary (CHO) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon, and, optionally, may contain enhancers, and various introns. This expression vector optionally contains sequences encoding all or part of a constant region, enabling an entire, or a part of, a heavy or light chain to be expressed. The gene construct can be introduced into eukaryotic host cells using conventional techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector encoding a polypeptide comprising an entire, or part of, a heavy chain or heavy chain variable region, and another expression vector encoding a polypeptide comprising an entire, or part of, a light chain or light chain variable region).

A polypeptide comprising an immunoglobulin heavy chain variable region or light chain variable region can be produced by growing (culturing) a host cell transfected with an expression vector encoding such variable region, under conditions that permit expression of the polypeptide. Following expression, the polypeptide can be harvested and purified or isolated using techniques known in the art, e.g., affinity tags such as glutathione-S-transferase (GST) and histidine tags.

A monoclonal antibody that binds human Notch3, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment of the antibody) can be harvested and purified or isolated using techniques known in the art, e.g., Protein A, Protein G, affinity tags such as glutathione-S-transferase (GST) and histidine tags. It is within ordinary skill in the art to express the heavy chain and the light chain from a single expression vector or from two separate expression vectors.

### III. Antibody Modifications

Methods for reducing or eliminating the antigenicity of antibodies and antibody fragments are known in the art. When administered to a human, the disclosed antibodies preferably are “humanized” to reduce or eliminate antigenicity in humans. Preferably, the humanized antibodies have the same, or substantially the same, affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments of the invention, the CDRs of the light and heavy chain variable regions of an anti-Notch3 antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains are said to be produced. See, e.g., U.S. Pat. No. 6,706,477 (Zauderer); U.S. Pat. No. 6,800,442 (Zauderer); and U.S. Pat. No. 6,872,518 (Zauderer).

Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection.

Another approach for modifying a mouse antibody into a form suitable for medical use in humans is HUMAN ENGINEERING™ technology, which is practiced commercially by XOMA (US) LLC. See, e.g., PCT Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

Any suitable approach, including any of the above approaches, can be used to reduce or eliminate human immunogenicity of an antibody of the invention.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

### IV. Use of Antibodies

Antibodies disclosed herein may be engineered (e.g., humanized) for administration to humans. Antibodies disclosed herein can be used to treat various forms of cancer, e.g., breast, ovarian, prostate, cervical, colorectal, lung, pancreatic, gastric, and head and neck cancers. The cancer cells are exposed to a therapeutically effective amount of the antibody so as to inhibit or reduce proliferation of the cancer cells. In some embodiments, the antibodies inhibit cancer cell proliferation by at least 40%, 50%, 60%, 70%, 80%, 90%, 95%, 98%, 99% or 100%.

In some embodiments, the disclosed antibodies may inhibit or reduce proliferation of a tumor cell by inhibiting binding of human Notch3 to a ligand, e.g., Jag1, Jag2, DLL1, and DLL4. The disclosed antibodies can be used in a method to inhibit tumor growth in a human patient. The method comprises administering to the patient a therapeutically effective amount of the antibody.

Cancers associated with Notch3 overexpression and/or activation include, but are not limited to, breast cancer, ovarian cancer, prostate cancer, cervical cancer, lung cancer, brain cancer (e.g., glioblastoma, astrocytoma, neuroblastoma), melanomas, gastrointestinal cancers (e.g., colorectal, pancreatic, and gastric), head and neck cancer, sarcomas (e.g. rhabdomyosarcoma, osteosarcoma), and hematopoietic cell cancers, (e.g., multiple myeloma, leukemia, e.g., precursor T acute lymphoblastic leukemia (T-ALL), precursor B acute lymphoblastic leukemia (B-ALL) and B-cell chronic lymphoblastic leukemia (B-CLL)).

As used herein, “treat”, “treating” and “treatment” mean the treatment of a disease in a mammal, e.g., in a human. This includes: (a) inhibiting the disease, i.e., arresting its development; and (b) relieving the disease, i.e., causing regression of the disease state.

Generally, a therapeutically effective amount of active component is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, and the disease being treated. Exemplary dosing frequencies are once per day, once per week and once every two weeks. A preferred route of administration is parenteral, e.g., intravenous infusion. Formulation of monoclonal antibody-based drugs is within ordinary skill in the art. In some embodiments, a monoclonal antibody is lyophilized, and then reconstituted in buffered saline, at the time of administration.

For therapeutic use, an antibody preferably is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing antibodies disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for monoclonal antibodies is IV infusion. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

### EXAMPLES

The following Examples are merely illustrative and are not intended to limit the scope or content of the invention in any way.

### Example 1: Production of Anti-hNotch3 Monoclonal Antibodies

Immunizations, fusions, and primary screens were conducted using conventional methods following the Repetitive Immunization Multiple Sites (RIMMS) protocol. Five AJ mice and five Balb/c mice were immunized with a protein containing amino acids 1-428 of human Notch3 fused to the Fc portion of human IgG. In addition, five AJ and five Balb/c mice were immunized with a concatemeric protein containing two repeats of the region of Notch3 comprising EGF-like domains 9-12. From each immunization strategy, two AJ mice and 2 Balb/c mice having sera displaying high binding to immunogen by Enzyme Linked ImmunoSorbent Assay (ELISA) were chosen for subsequent fusion. Spleens and lymph nodes from the selected mice were harvested. B cells were harvested and fused with a myeloma line. Fusion products from AJ mice and Balb/c mice were serially diluted in forty 96-well plates to near clonality. A total of 5,280 supernatants from the cell fusions were screened for binding to human Notch3 on the surface of CHO cells, using a Mesoscale electrochemiluminescence assay (MSD). In total, four hundred and twenty supernatants that bound human Notch3 in this assay were identified from the AJ and Balb/c fusions. These fusion products were further characterized by in vitro biochemical and cell-based assays, as discussed below. A panel of hybridomas was selected, the hybridomas were subcloned, and monoclonal hybridomas were expanded. Antibodies were expressed from the hybridoma cell lines and purified by affinity chromatography on Protein G resin under standard conditions.

### Example 2: Sequence Analysis of Anti-Notch3 Monoclonal Antibodies

The light chain isotype and heavy chain isotype of the monoclonal antibody, 4F11, in Example 1 was determined using the IsoStrip™ Mouse Monoclonal Antibody Isotyping Kit according to the manufacturer's instructions (Roche Applied Science, Indianapolis, IN). The antibody was determined to be kappa light chain and IgG1 heavy chain.

The heavy and light chain variable regions of the mouse monoclonal antibody were sequenced using 5′ RACE (Rapid Amplification of cDNA Ends). Total RNA was extracted from the hybridoma cell line using the RNeasy Miniprep kit according to the vendor's instructions (Qiagen, Valencia, CA). Full-length first strand cDNA containing 5′ ends was generated using the SMARTer™ RACE cDNA Amplification Kit (Clontech, Mountain View, CA) according to the manufacturer's instructions using random primers for 5′ RACE.

The variable regions of the light (kappa) and heavy (IgG1) chains were amplified by PCR, using KOD Hot Start Polymerase (EMD Chemicals, Gibbstown, NJ) according to the manufacturer's instructions. For amplification of 5′ cDNA ends in conjunction with the SMARTer™ RACE cDNA Amplification Kit, the Universal Primer Mix A primer (Clontech), a mix of 5′CTAATACGACTCACTATAGGGCAAGCAGTGGTATCAACGCAGAGT 3′ (SEQ ID NO: 13) and 5′ CTAATACGACTCACTATAGGGC 3′ (SEQ ID NO: 17), was used as a 5′ primer. The heavy chain variable region was amplified using the above 5′ primers and a 3′ IgG1 constant region specific primer, 5′ TATGCAAGGCTTACAACCACA 3′ (SEQ ID NO: 18). The kappa chain variable region was amplified with the above 5′ primers and a 3′ kappa constant region specific primer, 5′ CGACTGAGGCACCTCCAGATGTT 3′ (SEQ ID NO: 19).

Individual PCR products were isolated by agarose gel electrophoresis and purified using the Qiaquick Gel Purification kit according to the manufacturer's instructions (Qiagen). The PCR products were subsequently cloned into the pCR®4Blunt using the Zero Blunt® TOPO® PCR Cloning Kit according to the manufacturer's instructions (Invitrogen) and transformed into DH5-α bacteria (Invitrogen) through standard molecular biology techniques. Plasmid DNA isolated from transformed bacterial clones was sequenced using M13 Forward (5′ GTAAAACGACGGCCAGT 3′) (SEQ ID NO: 21) and M13 Reverse primers (5′ CAGGAAACAGCTATGACC 3′) (SEQ ID NO: 30) by Beckman Genomics (Danvers, MA), using standard dideoxy DNA sequencing methods to identify the sequence of the variable region sequences. The sequences were analyzed using Vector NTI software (Invitrogen) and the IMGT/V-Quest web server (available on the world wide web at imgt.cines.fr) to identify and confirm variable region sequences.

The nucleic acid sequences encoding and the protein sequences defining variable regions of the murine monoclonal antibodies are summarized below (amino terminal signal peptide sequences are not shown). CDR sequences (Kabat definition) are shown in bold and are underlined in the amino acid sequences.

| Nucleic Acid Sequence Encoding the Heavy Chain Variable Region of the 4F11 | |
|---|---|
| Antibody | |
| (SEQ ID NO: 1) | |
| 1 | gaagtgcagc tggtggagtc tgggggaggc ttagtgaagc ctggagggtc cctgaaactc |
| 61 | tcctgtgcag cctctggatt cgctttcagt agctatgaca tgtcttgggt tcgccagact |
| 121 | ccggagaaga ggctggagtg ggtcgcatac attagtcgtg gtggtggtag cacctactat |
| 181 | ccagacactg tgaagggccg attcaccatc tccagagaca atgccaagaa caccctgtac |
| 241 | ctgcaaatga gcagtctgaa gtctgaggac acagccatgt attactgtgg aagacatgct |
| 301 | actacggcct actggtactt cgatgtctgg ggcgcaggga ccacggtcac cgtctcctca |
| Protein Sequence Defining the Heavy Chain Variable Region of the 4F11 | |
| Antibody | |
| (SEQ ID NO: 2) | |
| 1 | evqlvesggg lvkpggslkl scaasgfafs sydmswvrqt pekrlewvay isrgggstyy |
| 61 | pdtvkgrfti srdnakntly lqmsslksed tamyycgrha ttaywyfdvw gagttvtvss |
| Nucleic Acid Sequence Encoding the Kappa Chain Variable Region of the 4F11 | |
| Antibody | |
| (SEQ ID NO: 3) | |
| 1 | gatgttgtga tgacccaaac tccactctcc ctgcctgtca gtcttggaga tcaagcctcc |
| 61 | atctcttgca gatctagtca gagccttgta cacactaatg gcaacaccta tttacattgg |
| 121 | tacctgcaga agccaggcca gtctccaaaa ctcctgatct acaaagtttc caaccgattt |
| 181 | tctggggtcc cagacaggtt cagtggcagt ggatcaggga cagatttcac actcaagatc |
| 241 | agcagagtgg aggctgagga tctgggagtt tatttctgct ctcaaagtac acatgttccg |
| 301 | tggacgttcg gtggaggcac caagctggaa atcaaa |
| Protein Sequence Defining the Kappa Chain Variable Region of the 4F11 | |
| Antibody | |
| (SEQ ID NO: 4) | |
| 1 | dvvmtqtpls lpvslgdqas iscrsscfslv htngntylhw ylqkpgqspk lliykvsnrf |
| 61 | sgvpdrfsgs gsgtdftlki srveaedlgv yfcsqsthvp wtfgggtkle ik |

Table 1 is a concordance chart showing the SEQ ID NO. of each sequence discussed in this Example.

TABLE 1

| SEQ. ID | |
|---|---|
| NO. | Nucleic Acid or Protein |
| 1 | 4F11 Heavy Chain Variable Region-nucleic acid |
| 2 | 4F11 Heavy Chain Variable Region-protein |
| 3 | 4F11 Light (kappa) Chain Variable Region-nucleic acid |
| 4 | 4F11 Light (kappa) Chain Variable Region-protein |
| 5 | 4F11 Heavy Chain CDR₁ |
| 6 | 4F11 Heavy Chain CDR₂ |
| 7 | 4F11 Heavy Chain CDR₃ |
| 8 | 4F11 Light (kappa) Chain CDR₁ |
| 9 | 4F11 Light (kappa) Chain CDR₂ |
| 10 | 4F11 Light (kappa) Chain CDR₃ |

Mouse monoclonal antibody heavy chain CDR sequences (Kabat, Chothia, and IMGT definitions) for 4F11 are shown in Table 2.

TABLE 2

| | CDR1 | CDR2 | CDR3 |
|---|---|---|---|
| Kabat | SYDMS | YISRGGGSTYYPDTVKG | HATTAYWYFDV |
| | (SEQ ID NO: 5) | (SEQ ID NO: 6) | (SEQ ID NO: 7) |
| Chothia | GFAFSSY | SRGGGS | HATTAYWYFDV |
| | (SEQ ID NO: 11) | (SEQ ID NO: 12) | (SEQ ID NO: 7) |
| IMGT | GFAFSSYD | ISRGGGST | GRHATTAYWYFDV |
| | (SEQ ID NO: 14) | (SEQ ID NO: 15) | (SEQ ID NO: 16) |

Mouse monoclonal antibody kappa light chain CDR sequences (Kabat, Chothia, and IMGT definitions) for 4F11 are shown in Table 3.

TABLE 3

| | CDR1 | CDR2 | CDR3 |
|---|---|---|---|
| Kabat/ | RSSQSLVHTNGNTYLH | KVSNRFS | SQSTHVPWT |
| Chothia | (SEQ ID NO: 8) | (SEQ ID NO: 9) | (SEQ ID NO: 10) |
| IMGT | QSLVHTNGNTY | KVS | SQSTHVPWT |
| | (SEQ ID NO: 20) | | (SEQ ID NO: 10) |

To create the complete heavy or kappa chain antibody sequences, each variable sequence above is combined with its respective constant region. For example, a complete heavy chain comprises a heavy variable sequence followed by the murine IgG1 heavy chain constant sequence, and a complete kappa chain comprises a kappa variable sequence followed by the murine kappa light chain constant sequence.

| Nucleic Acid Sequence Encoding the Murine IgG1 Heavy Chain Constant | |
|---|---|
| Region_ | |
| (SEQ ID NO: 22) | |
| 1 | gccaaaacga cacccccatc tgtctatcca ctggcccctg gatctgctgc ccaaactaac |
| 61 | tccatggtga ccctgggatg cctggtcaag ggctatttcc ctgagccagt gacagtgacc |
| 121 | tggaactctg gatccctgtc cagcggtgtg cacaccttcc cagctgtcct gcagtctgac |
| 181 | ctctacactc tgagcagctc agtgactgtc ccctccagca cctggcccag cgagaccgtc |
| 241 | acctgcaacg ttgcccaccc ggccagcagc accaaggtgg acaagaaaat tgtgcccagg |
| 301 | gattgtggtt gtaagccttg catatgtaca gtcccagaag tatcatctgt cttcatcttc |
| 361 | cccccaaagc ccaaggatgt gctcaccatt actctgactc ctaaggtcac gtgtgttgtg |
| 421 | gtagacatca gcaaggatga tcccgaggtc cagttcagct ggtttgtaga tgatgtggag |
| 481 | gtgcacacag ctcagacgca accccgggag gagcagttca acagcacttt ccgctcagtc |
| 541 | agtgaacttc ccatcatgca ccaggactgg ctcaatggca aggagttcaa atgcagggtc |
| 601 | aacagtgcag ctttccctgc ccccatcgag aaaaccatct ccaaaaccaa aggcagaccg |
| 661 | aaggctccac aggtgtacac cattccacct cccaaggagc agatggccaa ggataaagtc |
| 721 | agtctgacct gcatgataac agacttcttc cctgaagaca ttactgtgga gtggcagtgg |
| 781 | aatgggcagc cagcggagaa ctacaagaac actcagccca tcatggacac agatggctct |
| 841 | tacttcgtct acagcaagct caatgtgcag aagagcaact gggaggcagg aaatactttc |
| 901 | acctgctctg tgttacatga gggcctgcac aaccaccata ctgagaagag cctctcccac |
| 961 | tctcctggta aa |
| Protein Sequence Defining the Murine IgG1 Heavy Chain Constant Region | |
| (SEQ ID NO: 23) | |
| 1 | akttppsvyp lapgsaaqtn smvtlgclvk gyfpepvtvt wnsgslssgv htfpavlqsd |
| 61 | lytlsssvtv psstwpsetv tcnvahpass tkvdkkivpr dcgckpcict vpevssvfif |
| 121 | ppkpkdvlti tltpkvtcvv vdiskddpev qfswfvddve vhtaqtqpre eqfnstfrsv |
| 181 | selpimhqdw lngkefkcrv nsaafpapie ktisktkgrp kapqvytipp pkeqmakdkv |
| 241 | sltcmitdff peditvewqw ngqpaenykn tqpimdtdgs yfvysklnvq ksnweagntf |
| 301 | tcsvlheglh nhhtekslsh spgk |
| Nucleic Acid Sequence Encoding the Murine Kappa Light Chain Constant | |
| Region_ | |
| (SEQ ID NO: 24) | |
| 1 | cgggctgatg ctgcaccaac tgtatccatc ttcccaccat ccagtgagca gttaacatct |
| 61 | ggaggtgcct cagtcgtgtg cttcttgaac aacttctacc ccaaagacat caatgtcaag |
| 121 | tggaagattg atggcagtga acgacaaaat ggcgtcctga acagttggac tgatcaggac |
| 181 | agcaaagaca gcacctacag catgagcagc accctcacgt tgaccaagga cgagtatgaa |
| 241 | cgacataaca gctatacctg tgaggccact cacaagacat caacttcacc cattgtcaag |
| 301 | agcttcaaca ggaatgagtg t |
| Protein Sequence Defining the Murine Kappa Light Chain Constant Region | |
| (SEQ ID NO: 25) | |
| 1 | radaaptvsi fppsseqlts ggasvvcfln nfypkdinvk wkidgserqn gvlnswtdqd |
| 61 | skdstysmss tltltkdeye rhnsytceat hktstspivk sfnrnec |

The following sequences represent the actual or contemplated full length heavy and light chain sequences (i.e., containing both the variable and constant regions sequences) for the 4F11 antibody described in this Example. Signal sequences for proper secretion of the antibodies (e.g., signal sequences at the 5′ end of the DNA sequences or the amino terminal end of the protein sequences) are not shown in the full length heavy and light chain sequences disclosed herein and are not included in the final secreted protein. Also not shown are stop codons for termination of translation required at the 3′ end of the DNA sequences. It is within ordinary skill in the art to select a signal sequence and/or a stop codon for expression of the disclosed full length IgG heavy chain and light chain sequences. It is also contemplated that the variable region sequences can be ligated to other constant region sequences to produce active full length IgG heavy and light chains.

| Nucleic Acid Sequence Encoding the Full Length Heavy Chain Sequence | |
|---|---|
| (Heavy Chain Variable Region and IgG1 Constant Region) of 4F11 | |
| (SEQ ID NO: 26) | |
| 1 | gaagtgcagc tggtggagtc tgggggaggc ttagtgaagc ctggagggtc cctgaaactc |
| 61 | tcctgtgcag cctctggatt cgctttcagt agctatgaca tgtcttgggt tcgccagact |
| 121 | ccggagaaga ggctggagtg ggtcgcatac attagtcgtg gtggtggtag cacctactat |
| 181 | ccagacactg tgaagggccg attcaccatc tccagagaca atgccaagaa caccctgtac |
| 241 | ctgcaaatga gcagtctgaa gtctgaggac acagccatgt attactgtgg aagacatgct |
| 301 | actacggcct actggtactt cgatgtctgg ggcgcaggga ccacggtcac cgtctcctca |
| 361 | gccaaaacga cacccccatc tgtctatcca ctggcccctg gatctgctgc ccaaactaac |
| 421 | tccatggtga ccctgggatg cctggtcaag ggctatttcc ctgagccagt gacagtgacc |
| 481 | tggaactctg gatccctgtc cagcggtgtg cacaccttcc cagctgtcct gcagtctgac |
| 541 | ctctacactc tgagcagctc agtgactgtc ccctccagca cctggcccag cgagaccgtc |
| 601 | acctgcaacg ttgcccaccc ggccagcagc accaaggtgg acaagaaaat tgtgcccagg |
| 661 | gattgtggtt gtaagccttg catatgtaca gtcccagaag tatcatctgt cttcatcttc |
| 721 | cccccaaagc ccaaggatgt gctcaccatt actctgactc ctaaggtcac gtgtgttgtg |
| 781 | gtagacatca gcaaggatga tcccgaggtc cagttcagct ggtttgtaga tgatgtggag |
| 841 | gtgcacacag ctcagacgca accccgggag gagcagttca acagcacttt ccgctcagtc |
| 901 | agtgaacttc ccatcatgca ccaggactgg ctcaatggca aggagttcaa atgcagggtc |
| 961 | aacagtgcag ctttccctgc ccccatcgag aaaaccatct ccaaaaccaa aggcagaccg |
| 1021 | aaggctccac aggtgtacac cattccacct cccaaggagc agatggccaa ggataaagtc |
| 1081 | agtctgacct gcatgataac agacttcttc cctgaagaca ttactgtgga gtggcagtgg |
| 1141 | aatgggcagc cagcggagaa ctacaagaac actcagccca tcatggacac agatggctct |
| 1201 | tacttcgtct acagcaagct caatgtgcag aagagcaact gggaggcagg aaatactttc |
| 1261 | acctgctctg tgttacatga gggcctgcac aaccaccata ctgagaagag cctctcccac |
| 1321 | tctcctggta aa |
| Protein Sequence Defining the Full Length Heavy Chain Sequence (Heavy | |
| Chain Variable Region and IgG1 Constant Region) of 4F11 | |
| (SEQ ID NO: 27) | |
| 1 | evqlvesggg lvkpggslkl scaasgfafs sydmswvrqt pekrlewvay isrgggstyy |
| 61 | pdtvkgrfti srdnakntly lqmsslksed tamyycgrha ttaywyfdvw gagttvtvss |
| 121 | akttppsvyp lapgsaaqtn smvtlgclvk gyfpepvtvt wnsgslssgv htfpavlqsd |
| 181 | lytlsssvtv psstwpsetv tcnvahpass tkvdkkivpr dcgckpcict vpevssvfif |
| 241 | ppkpkdvlti tltpkvtcvv vdiskddpev qfswfvddve vhtaqtqpre eqfnstfrsv |
| 301 | selpimhqdw lngkefkcrv nsaafpapie ktisktkgrp kapqvytipp pkeqmakdkv |
| 361 | sltcmitdff peditvewqw ngqpaenykn tqpimdtdgs yfvysklnvq ksnweagntf |
| 421 | tcsvlheglh nhhtekslsh spgk |
| Nucleic Acid Sequence Encoding the Full Length Light Chain Sequence | |
| (Kappa Chain Variable Region and Constant Region) of 4F11 | |
| (SEQ ID NO: 28) | |
| 1 | gatgttgtga tgacccaaac tccactctcc ctgcctgtca gtcttggaga tcaagcctcc |
| 61 | atctcttgca gatctagtca gagccttgta cacactaatg gcaacaccta tttacattgg |
| 121 | tacctgcaga agccaggcca gtctccaaaa ctcctgatct acaaagtttc caaccgattt |
| 181 | tctggggtcc cagacaggtt cagtggcagt ggatcaggga cagatttcac actcaagatc |
| 241 | agcagagtgg aggctgagga tctgggagtt tatttctgct ctcaaagtac acatgttccg |
| 301 | tggacgttcg gtggaggcac caagctggaa atcaaacggg ctgatgctgc accaactgta |
| 361 | tccatcttcc caccatccag tgagcagtta acatctggag gtgcctcagt cgtgtgcttc |
| 421 | ttgaacaact tctaccccaa agacatcaat gtcaagtgga agattgatgg cagtgaacga |
| 481 | caaaatggcg tcctgaacag ttggactgat caggacagca aagacagcac ctacagcatg |
| 541 | agcagcaccc tcacgttgac caaggacgag tatgaacgac ataacagcta tacctgtgag |
| 601 | gccactcaca agacatcaac ttcacccatt gtcaagagct tcaacaggaa tgagtgt |
| Protein Sequence Defining the Full Length Light Chain Sequence (Kappa | |
| Chain Variable Region and Constant Region) of 4F11 | |
| (SEQ ID NO: 29) | |
| 1 | dvvmtqtpls lpvslgdqas iscrssqslv htngntylhw ylqkpgqspk lliykvsnrf |
| 61 | sgvpdrfsgs gsgtdftlki srveaedlgv yfcsqsthvp wtfgggtkle ikradaaptv |
| 121 | sifppsseql tsggasvvcf lnnfypkdin vkwkidgser qngvlnswtd qdskdstysm |
| 181 | sstltltkde yerhnsytce athktstspi vksfnrnec |

Table 4 is a concordance chart showing the correspondence between the full length sequences of the antibodies discussed in this Example with those presented in the Sequence Listing.

TABLE 4

| SEQ ID | |
|---|---|
| NO. | Nucleic Acid or Protein |
| 26 | 4F11 Heavy Variable + IgG1 Constant-nucleic acid |
| 27 | 4F11 Heavy Variable + IgG1 Constant-protein |
| 28 | 4F11 Kappa Variable + Constant-nucleic acid |
| 29 | 4F11 Kappa Variable + Constant-protein |

### Example 3: Binding Affinities

The binding affinity and kinetics of binding of antibody 4F11 to recombinant human Notch3 extracellular domain (containing EGF like domains 1-11) Fc fusion protein (rhNotch3-Fc (R&D Systems, Inc., Minneapolis, MN)) were measured by surface plasmon resonance using a Biacore® T100 instrument (GE Healthcare, Piscataway, NJ).

Rabbit anti-mouse IgGs (GE Healthcare) were immobilized on carboxymethylated dextran CM4 sensor chips (GE Healthcare) by amine coupling, according to a standard protocol. Analyses were performed at 25° C. and 37° C. using PBS containing 0.05% surfactant P20 as running buffer. The antibody was captured in individual flow cells at a flow rate of 10 μl/minute. Injection time was varied for each antibody to yield an Rmax between 30 and 60 resonance units (RU). Buffer or rhNotch3-Fc diluted in running buffer was injected sequentially over a reference surface (no antibody captured) and the active surface (antibody to be tested) for 240 seconds at 60 μl/minute. The dissociation phase was monitored for up to 1500 seconds. The surface was then regenerated with two 60-second injections of 10 mM Glycine-HCl, pH 2.25, at a flow rate of 30 μl/minute. The rhNotch3-Fc concentration range tested was 100 nM to 3.125 nM (2 fold dilution).

Kinetic parameters were determined using the kinetic function of the BIAevaluation software (GE Healthcare) with double reference subtraction. Kinetic parameters for the antibody, kₐ (association rate constant), k_{d} (dissociation rate constant) and K_{D} (equilibrium dissociation constant) were determined. Kinetic values of the monoclonal antibody 4F11 on rhNotch3-Fc at 25° C. and 37° C. are summarized in Table 5.

TABLE 5
Antibody Binding to rhNotch3-Fc

| Temperature | kₐ (1/Ms) | k_{d} (1/s) | K_{D} (M) | n |
|---|---|---|---|---|
| 25° C. | 7.0E+04 | 4.8E−05 | 8.23E−10 | 3 |
| 37° C. | 7.5E+04 | 5.7E−05 | 7.8E−10 | 3 |

The data in Table 5 demonstrate that the antibody 4F11 binds rhNotch3-Fc with a K_{D} of about 1 nM, 900 pM, 850 pM, 800 pM, 750 pM, 700 pM, 650 pM, 600 pM, 500 pM, 400 pM, 300 pM, 250 pM, or 200 pM or less.

Binding to cell surface human Notch3 by the antibody 4F11 was measured at 4° C., using Fluorescence Activated Cell Sorting (FACS). CHO N3 (Flp-In-CHO cells (Invitrogen) stably transfected with human Notch3), HCC2429 cells, and RL-952 cells expressing human Notch3 were washed once with PBS containing calcium chloride and magnesium chloride (Invitrogen) and harvested using cell dissociation buffer (Invitrogen). Cells were washed a second time with PBS and resuspended in FACS buffer (PBS with 0.5% BSA (Sigma-Aldrich)) for a final cell concentration of 250,000 cells per well into a 96-well v-bottom plate. Purified antibodies were diluted in FACS buffer over a concentration range of 100 nM to 0.1 nM. Cells were then incubated at 4° C. with 100 μl of antibody for one hour, washed with FACS buffer twice, and resuspended in 100 μl of goat-anti mouse PE-conjugated antibody (Jackson Immuno Research). Cells were incubated at 4° C. for 30 minutes in the dark, washed once with FACS buffer, and then analyzed using a Beckman Coulter Cytomics FC 500 instrument. The geometric mean of the florescent intensity was then calculated for each antibody concentration. These values were then entered into Prism software (GraphPad, La Jolla, Ca) and used to generate a binding curve by plotting geometric mean versus antibody concentration. From the binding curve, the following equation was used to calculate the K_{D} and K_{D} range of 4F11 binding to human Notch3 on the cell surface of the three cell lines.

$$One site binding ( hyperbola ) \qquad Equation$$

$$Y = {B}_{max} ⋆ X / ( {K}_{D} + X )$$

*describes the binding of a ligand to a receptor that follows the law of mass action. Bₘₐₓ is the maximal binding, and K_{D} is the concentration of ligand required to reach half-maximal binding.

Results are summarized in Table 6.

TABLE 6

| | K_{D} (nM) | K_{D} Range (nM) |
|---|---|---|
| CH0 N3 | 0.36 | 0.21 to 0.51 |
| HCC2429 | 2.58 | 1.21 to 3.95 |
| RL-952 | 0.9633 | 0.7168 to 1.210 |

The results in Table 6 demonstrate that the antibody 4F11 binds cell surface Notch3 with a K_{D} of about 1 nM, 750 pM, 650 pM, 600 pM, 500 pM, 400 pM, 300 pM, 250 pM, or 200 pM or less.

### Example 4: Binding Specificity

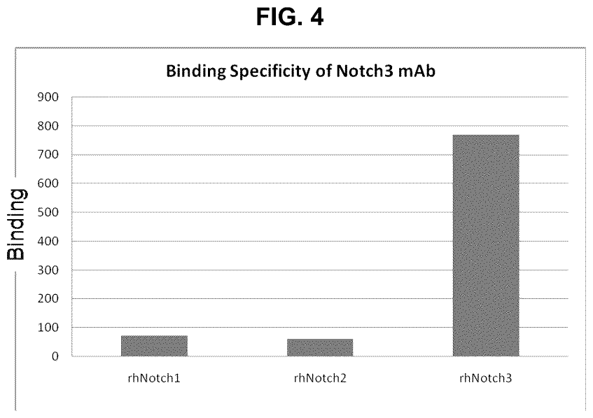

Antibody 4F11 was tested for binding to human Notch1, human Notch2, or human Notch3 proteins. Binding measurements were made by biolayer interferometry (BLI), using a FortéBio Octet® QK instrument (FortéBio, Menlo Park, CA). Anti-human-Fc sensors were soaked in PBS containing 1 mg/ml BSA for five minutes prior to binding of antibodies. Then the following proteins (400 nM, in PBS containing 1 mg/ml BSA) were allowed to bind to the sensors: rhNotch1-Fc (R&D Systems, Minneapolis, MN; Cat. No. 3647-TK-050), rhNotch2-Fc (R&D Cat. No. 3735-NT-050), rhNotch3-Fc (R&D Cat. No. 1559-NT-050), or rmNotch3-Fc (R&D Cat. No. 1308-NT-050). Notch protein bound sensors were immersed in antibody solution (50 μg/ml) to allow binding of antibody to the Notch protein. Binding was detected by shifts in the interference pattern. These results demonstrated that the antibodies bind specifically to human Notch3 protein, but do not bind to human Notch2 or human Notch1 protein (FIG. 4).

Stable cells lines expressing Notch receptors were produced by transfecting FlpIn™ CHO or FlpIn™ 293 cells (Life Technologies, Grand Island, NY) with full length human Notch1, Notch2, Notch3, or Notch4 cDNAs cloned into the pcDNA5FRT vector using Lipofectamine 2000 (Life Technologies) according to the manufacturer's protocol. Twenty-four hours after transfection, CHO cells were split into F12 media containing 10% FBS, 2 mM L-Glutamine and 700 μg/ml hygromycin B (Sigma-Aldrich, St. Louis, MO) to select for transfected cells. 293 cells were split into DMEM media containing 10% FBS, 2 mM L-glutamine and 200 μg/ml hygromycin B. Expression of Notch receptors was confirmed by FACS analysis using anti-human Notch1 PE (BioLegend, San Diego, CA), anti-Notch2 PE (eBioscience, San Diego, CA), or anti-human Notch3 PE (BioLegend, San Diego, CA).

To determine specificity of binding to cell surface Notch proteins, antibody 4F11 was tested for binding to human Notch1, human Notch2, human Notch3 and human Notch4 expressed on the surface of CHO cells using electrochemiluminescence (Meso Scale Discovery). A CHO line lacking any human Notch protein was also produced for use as a negative control. Cells were grown under standard conditions (37° C., F12+10% FBS). For binding studies, cells were washed in PBS containing calcium and magnesium, and removed from the plate by treatment with Cell Dissociation Buffer (Life Technologies) for ten minutes at 37° C.

Cells were seeded at a density of 30,000 cells per well, in hybridoma media, instandard 96-well binding plates (Meso Scale Discovery, Cat. No. L15XA-6). Cells were incubated for one hour at 37° C. Antibodies or control IgG were added at 5 μg/ml, in 50 μl hybridoma media, and incubated for one hour at 37° C. The plates were washed twice with PBS containing 3% BSA. Binding of the antibodies to cell surface was detected using 2 μg/ml of MSD anti-mouse IgG secondary antibody (Meso Scale Discovery, Cat. No. R32AC-1) for one hour at 4° C. Plates were washed twice with PBS containing 3% BSA, and 150 μl of read buffer (Meso Scale Discovery Cat. No. R92TC-1) was added. The plates were analyzed on a Sector Imager 2400 instrument (Meso Scale Discovery). This analysis showed that antibody 4F11 binds to human Notch3 displayed on the surfaces of cells, but does not bind to human Notch1, Notch2, or Notch4 displayed on the surfaces of cells. The 4F11 antibody also does not bind CHO-EV (empty vector) cells that express endogenous hamster Notch proteins. These results indicated that antibody 4F11 binds specifically to human Notch3 protein displayed on a cell surface in vitro.

### Example 5: Inhibition of Notch3-Ligand Binding

The 4F11 antibody was tested for its ability to inhibit the binding of rhNotch3 binding to human Jag1, Jag2, DLL1 and DLL4. Binding measurements were made by bio-layer interferometry (BLI), using a FortéBio Octet® QK instrument (FortéBio, Menlo Park, CA). The ligands tested were rhJag1-Fc (R&D Cat. No. 1277-JG-050), rhJag2-Fc (R&D Cat. No. 1726-JG-050), rhDLL1-Fc (R&D Cat. No. 5026-DL-050), and His tagged rhDLL4 (R&D Cat. No. 1506-D4-050).

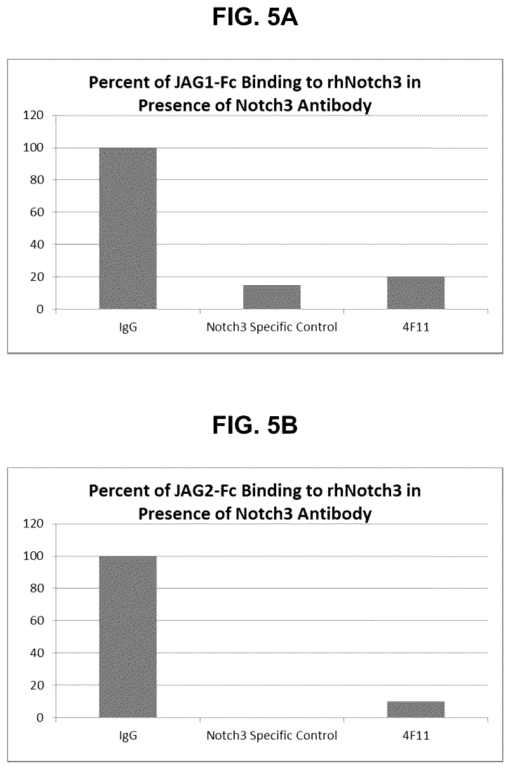

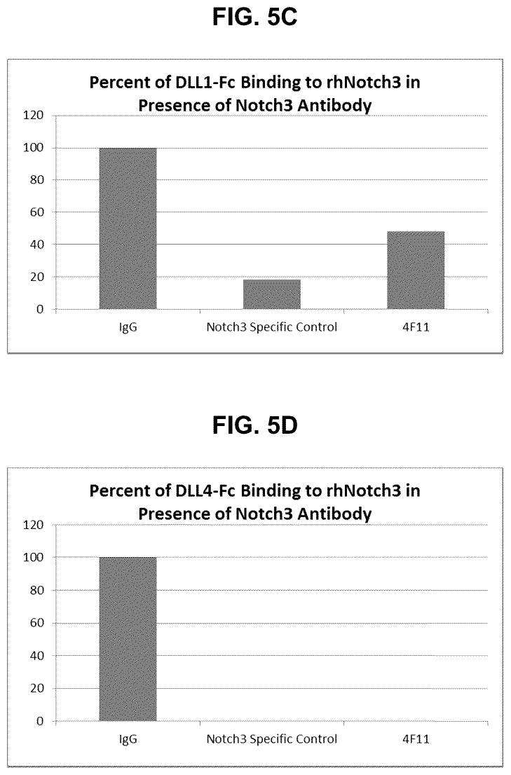

To determine the degree of inhibition of Notch3-ligand binding by antibody 4F11, the Octet sensors were loaded with recombinant human Notch3, and the antibody was allowed to bind, as described in Example 4. In positive control samples, a commercially available Notch3 polyclonal antibody capable of blocking ligand binding to recombinant human Notch3 (Notch3 Specific Control) was used instead of the 4F11 antibody. Then sensors were immersed in 500 μg/ml human IgG, to block non-specific binding. Ligands were prepared at a concentration of 400 nM in PBS containing 3% BSA, and were allowed to bind. The on-rate and off-rate for ligand binding were detected using the Octet QK instrument and software. The 4F11 antibody blocked binding of all four ligands to rhNotch3-Fc (FIG. 5A-5D).

### Example 6: Inhibition of Ligand-Induced Notch3 ICD Cleavage

Activation of Notch receptors results in cleavage of the Notch intracellular domain (NICD), which can be detected by Western blot. The effect of antibody 4F11 on the activation of Notch3 ICD cleavage was tested.

To create soluble Notch ligands, PCR was used to amplify sequences corresponding to the extracellular domains of human Jag1 or human Jag2 cDNA and fuse them in-frame to the coding sequence of human IgG Fc. This construct was then subcloned into the pEE14.4 expression vector (Lonza), transfected into CHOK1SV cells, and selected to produce stable cell lines that secrete hJag1-hFc or hJag2-hFc fusion protein. 96-well Immunosorp ELISA plates (Nalgene Nunc, Rochester, NY) were coated with 5 μg/ml anti-human Fc (Jackson ImmunoResearch, West Grove, PA) overnight at 4° C. After washing wells with PBS/0.5% BSA, 5 μg/ml of soluble hJag1-hFc fusion protein was added and allowed to bind at room temperature for two hours. Unbound protein was removed by washing with PBS/0.5% BSA. FlpIn™ 293 cells engineered to express hNotch3 (as described in Example 4) were plated on Jag1-hFc ligand or hFc in the presence of 10 μg/ml 4F11 or mIgG control antibody. Cells were lysed 24 hours later in RIPA buffer (Boston BioProducts, Ashland, MA) containing protease inhibitors. Induction of cleaved NICD was detected by probing the blot with a Notch3 antibody against the C-terminus (Cell Signaling, Danvers, MA) that detects both full length protein and the cleaved ICD. Ligand-induced activation and Notch3 ICD cleavage was inhibited by antibody 4F11.

### Example 7: Inhibition of Notch3-Dependent Transcription

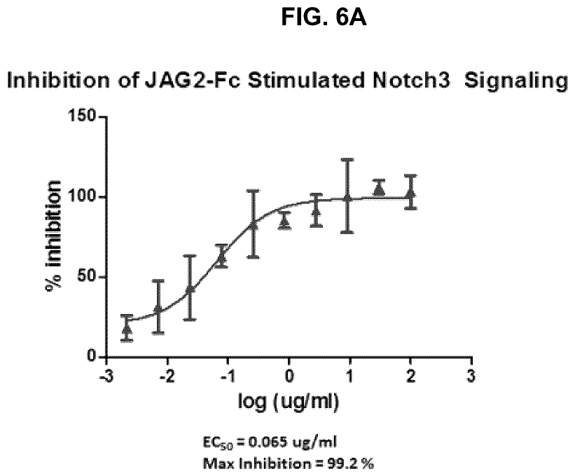

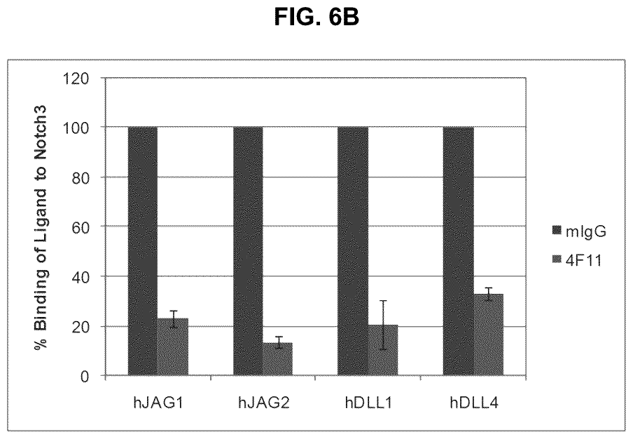

Reporter cell lines dependent upon Notch3 were produced by lentiviral introduction of a RBP-Jκ-dependent luciferase reporter gene (SABiosciences, Frederick, MD) into 293-FlpIn Notch3 cells, RL95-2 endometrial cancer cells, HCC1143 breast cancer cells, and MDA-MB-468 breast cancer cells. To activate Notch3-dependent signaling and transcription, cells were plated on ligand-coated wells prepared as described in Example 6. Cells were pre-incubated with a 3-fold dilution series of Notch3 antibody concentrations ranging from 0-300 μg/ml, for one hour at 37° C., before seeding 100 μl of the suspension into 96-well plates coated with ligand or hFc. Cells were incubated in ligand-coated or human-Fc-coated wells for four or twenty-four hours at 37° C., in 5% CO₂. Next, 100 μl of Promega Bright Glo™ (Promega, Madison, WI) was added to each well. The reaction was allowed to proceed for five minutes in the dark, and then the entire 200 μl volume was transferred to white walled plates and read using a luminometer. Polyclonal antibodies against Notch1 (AF1057, R&D Systems), Notch2 (AF1190, R&D Systems) or Notch3 (AF1559, R&D Systems) were used as controls to confirm that ligand-stimulated reporter activity in each cell line was specifically dependent upon the introduced Notch receptor. As shown in FIG. 6A, Notch3 antibody 4F11 inhibited Notch3-dependent transcription stimulated by the ligand Jag2. Activation of Notch3-dependent transcription by each of the ligands Jag1, Jag2, DLL1, and DLL4 was also inhibited by the Notch3 antibody 4F11 (FIG. 6B and Table 7).

TABLE 7

| Ligand | Jag1 | Jag2 | DLL1 | DLL4 |
|---|---|---|---|---|
| EC₅₀ | 2.6 nM | 0.4 nM | 6.0 nM | 4.7 nM |
| Maximum | 92% | 99% | 100% | 73% |
| Inhibition | | | | |

### Example 8: Humanization of Anti-Notch3 Antibodies

This Example describes the humanization and chimerization of the anti-human Notch3 antibody 4F11, and the characterization of the resulting humanized antibodies. The humanized anti-Notch3 antibodies were designed, affinity matured by targeted CDR mutagenesis, and optimized using methods known in the art. The amino acid sequences were converted to codon-optimized DNA sequences and synthesized to include (in the following order): 5′ HindIII restriction site, Kozak consensus sequence, amino terminal signal sequence, humanized variable region, human IgG1 or Kappa constant region, stop codon, and a 3′ EcoRI restriction site.

Chimeric (murine variable region and human constant region) 4F11 heavy (human IgG1) and light (human Kappa) chains were also constructed. To generate chimeric antibodies, the murine variable regions were fused to the human constant region, and codon-optimized DNA sequences were synthesized, including (in the following order): 5′ HindIII restriction site, Kozak consensus sequence, amino terminal signal sequence, mouse variable region, human IgG1 or Kappa constant region, stop codon, and 3′ EcoRI restriction site.

The humanized and chimeric heavy chains were subcloned into pEE6.4 (Lonza, Basel, Switzerland) via HindIII and EcoRI sites using In-Fusion™ PCR cloning (Clontech, Mountain View, CA). The humanized and chimeric Kappa light chains were subcloned into pEE14.4 (Lonza) via HindIII and EcoRI sites using In-Fusion™ PCR cloning.

Humanized antibody chains or chimeric antibody chains were transiently transfected into 293T cells to produce antibody. Antibody was either purified or used in cell culture media supernatant for subsequent in vitro analysis. Binding of the chimeric and humanized antibodies to Notch3 was measured as described below. The results are summarized in Tables 14-16.

Exemplary combinations of the chimeric or humanized 4F11 immunoglobulin heavy chain and immunoglobulin light chain variable regions is set forth below in Table 8.

TABLE 8

| Antibody | | |
|---|---|---|
| Name | Heavy Chain Variable Region | Light Chain Variable Region |
| Hu4F11-1 | Ch4F11 Chimeric Heavy | Ch4F11 Chimeric Kappa |
| | (SEQ ID NO: 2) | (SEQ ID NO:4) |
| Hu4F11-10 | Sh4F11 Hv3-23 Heavy | Ch4F11 Chimeric Kappa |
| | (SEQ ID NO: 34) | (SEQ ID NO:4) |
| Hu4F11-18 | Ch4F11 Chimeric Heavy | Hu4F11 Kv2D-29 Kappa |
| | (SEQ ID NO: 2) | (SEQ ID NO:44) |
| Hu4F11-32 | Sh4F11 Hv3-23 Heavy | Hu4F11 Kv2D-29 Kappa |
| | (SEQ ID NO: 34) | (SEQ ID NO:44) |
| Hu4F11-69 | Sh4F11 Hv3-23 A28T S31N T62S Heavy | Hu4F11 Kv2D-29 N28H Kappa |
| | (SEQ ID NO: 40) | (SEQ ID NO:46) |
| Hu4F11-70 | Sh4F11 Hv3-23 A28T S31H T62S Heavy | Hu4F11 Kv2D-29 N28H Kappa |
| | (SEQ ID NO: 36) | (SEQ ID NO:46) |
| Hu4F11-71 | Sh4F11 Hv3-23 A28T T62S Heavy | Hu4F11 Kv2D-29 N28H Kappa |
| | (SEQ ID NO: 42) | (SEQ ID NO:46) |
| Hu4F11-72 | Sh4F11 Hv3-23 S31H T62S Heavy | Hu4F11 Kv2D-29 N28H Kappa |
| | (SEQ ID NO: 38) | (SEQ ID NO:46) |
| Hu4F11-73 | Sh4F11 Hv3-23 A28T S31N T62S Heavy | Hu4F11 Kv2D-29 N28Y Kappa |
| | (SEQ ID NO: 40) | (SEQ ID NO:50) |
| Hu4F11-74 | Sh4F11 Hv3-23 A28T S31H T62S Heavy | Hu4F11 Kv2D-29 N28Y Kappa |
| | (SEQ ID NO: 36) | (SEQ ID NO:50) |
| Hu4F11-75 | Sh4F11 Hv3-23 A28T T62S Heavy | Hu4F11 Kv2D-29 N28Y Kappa |
| | (SEQ ID NO: 42) | (SEQ ID NO:50) |
| Hu4F11-76 | Sh4F11 Hv3-23 S31H T62S Heavy | Hu4F11 Kv2D-29 N28Y Kappa |
| | (SEQ ID NO: 38) | (SEQ ID NO:50) |
| Hu4F11-77 | Sh4F11 Hv3-23 A28T S31N T62S Heavy | Hu4F11 Kv2D-29 N28Q Kappa |
| | (SEQ ID NO: 40) | (SEQ ID NO:48) |
| Hu4F11-78 | Sh4F11 Hv3-23 A28T S31H T62S Heavy | Hu4F11 Kv2D-29 N28Q Kappa |
| | (SEQ ID NO: 36) | (SEQ ID NO:48) |
| Hu4F11-79 | Sh4F11 Hv3-23 A28T T62S Heavy | Hu4F11 Kv2D-29 N28Q Kappa |
| | (SEQ ID NO: 42) | (SEQ ID NO:48) |
| Hu4F11-80 | Sh4F11 Hv3-23 S31H T62S Heavy | Hu4F11 Kv2D-29 N28Q Kappa |
| | (SEQ ID NO: 38) | (SEQ ID NO:48) |

The nucleic acid sequences and the encoded protein sequences defining variable regions of the chimeric and humanized 4F11 antibodies are summarized below (amino terminal signal peptide sequences are not shown). CDR sequences (Kabat definition) are shown in bold and are underlined in the amino acid sequences.

| Nucleic Acid Sequence Encoding the Ch4F11 Chimeric Heavy Chain Variable | |
|---|---|
| Region | |
| (SEQ ID NO: 31) | |
| 1 | gaggtacagc ttgtcgagtc gggaggagga ttggtaaaac cgggtgggtc actcaaattg |
| 61 | tcgtgtgcgg cgtcgggatt tgcgttttcg tcgtatgata tgtcgtgggt gcgccagacg |
| 121 | ccggaaaaac gattggaatg ggtcgcgtat atctcccgag ggggaggttc gacatactat |
| 181 | cccgacacgg tcaaagggcg cttcacgatt tcacgggaca atgcgaaaaa cacgctttat |
| 241 | cttcaaatgt cgtcgttgaa atcggaagat accgcgatgt attactgcgg gaggcatgcg |
| 301 | acgacggcgt attggtattt cgatgtgtgg ggagccggaa cgacggtgac ggtgtcgtcg |
| Protein Sequence Defining the Ch4F11 Chimeric Heavy Chain Variable Region | |
| (SEQ ID NO: 2) | |
| 1 | evqlvesggg lvkpggslkl scaasgfafs sydmswvrqt pekrlewvay isrgggstyy |
| 61 | pdtvkgrfti srdnakntly lqmsslksed tamyycgrha ttaywyfdvw gagttvtvss |
| Nucleic Acid Sequence Encoding the Sh4F11 Hv3-23 Heavy Chain Variable | |
| Region | |
| (SEQ ID NO: 33) | |
| 1 | gaagtacagt tgttggagtc aggaggaggg ttggtccagc cgggtggatc gttgcggctt |
| 61 | tcgtgtgcgg cgtcgggatt cgcgttttca tcgtatgaca tgtcgtgggt gaggcaggca |
| 121 | ccggggaaag ggcttgaatg ggtatcgtac atttcgagag ggggaggatc gacgtattac |
| 181 | ccggatacgg tgaaaggaag gtttacgatc tcgcgcgaca attcaaagaa tacgctttat |
| 241 | cttcagatga actcgctccg agcggaagat acggcggtat actattgcgg tcgccatgcg |
| 301 | acgacggcgt attggtattt cgatgtgtgg ggacaaggga cgatggtcac ggtgtcgtcg |
| Protein Sequence Defining the Sh4F11 Hv3-23 Heavy Chain Variable Region | |
| (SEQ ID NO: 34) | |
| 1 | evqllesggg lvqpggslrl scaasgfafs sydmswvrqa pgkglewvsy isrgggstyy |
| 61 | pdtvkgrfti srdnskntly lqmnslraed tavyycgrha ttaywyfdvw gqgtmvtvss |
| Nucleic Acid Sequence Encoding the Sh4F11 Hv3-23 A28T S31H T62S Heavy | |
| Chain Variable Region | |
| (SEQ ID NO: 35) | |
| 1 | gaagtacagt tgttggagtc aggaggaggg ttggtccagc cgggtggatc gttgcggctt |
| 61 | tcgtgtgcgg cgtcgggatt caccttttca cactatgaca tgtcgtgggt gaggcaggca |
| 121 | ccggggaaag ggcttgaatg ggtatcgtac atttcgagag ggggaggatc gacgtattac |
| 181 | ccggattccg tgaaaggaag gtttacgatc tcgcgcgaca attcaaagaa tacgctttat |
| 241 | cttcagatga actcgctccg agcggaagat acggcggtat actattgcgg tcgccatgcg |
| 301 | acgacggcgt attggtattt cgatgtgtgg ggacaaggga cgatggtcac ggtgtcgtcg |
| Protein Sequence Defining the Sh4F11 Hv3-23 A28T S31H T62S Heavy Chain | |
| Variable Region | |
| (SEQ ID NO: 36) | |
| 1 | evqllesggg lvqpggslrl scaasgftfs hydmswvrqa pgkglewvsy isrgggstyy |
| 61 | pdsvkgrfti srdnskntly lqmnslraed tavyycgrha ttaywyfdvw gqgtmvtvss |
| Nucleic Acid Sequence Encoding the Sh4F11 Hv3-23 S31H T62S Heavy Chain | |
| Variable Region | |
| (SEQ ID NO: 37) | |
| 1 | gaagtacagt tgttggagtc aggaggaggg ttggtccagc cgggtggatc gttgcggctt |
| 61 | tcgtgtgcgg cgtcgggatt cgcgttttca cactatgaca tgtcgtgggt gaggcaggca |
| 121 | ccggggaaag ggcttgaatg ggtatcgtac atttcgagag ggggaggatc gacgtattac |
| 181 | ccggattccg tgaaaggaag gtttacgatc tcgcgcgaca attcaaagaa tacgctttat |
| 241 | cttcagatga actcgctccg agcggaagat acggcggtat actattgcgg tcgccatgcg |
| 301 | acgacggcgt attggtattt cgatgtgtgg ggacaaggga cgatggtcac ggtgtcgtcg |
| Protein Sequence Defining the Sh4F11 Hv3-23 S31H T62S Heavy Chain | |
| Variable Region | |
| (SEQ ID NO: 38) | |
| 1 | evqllesggg lvqpggslrl scaasgfafs hydmswvrqa pgkglewvsy isrgggstyy |
| 61 | pdsvkgrfti srdnskntly lqmnslraed tavyycgrha ttaywyfdvw gqgtmvtvss |
| Nucleic Acid Sequence Encoding the Sh4F11 Hv3-23 A28T S31N T62S Heavy | |
| Chain Variable Region | |
| (SEQ ID NO: 39) | |
| 1 | gaagtacagt tgttggagtc aggaggaggg ttggtccagc cgggtggatc gttgcggctt |
| 61 | tcgtgtgcgg cgtcgggatt caccttttca aactatgaca tgtcgtgggt gaggcaggca |
| 121 | ccggggaaag ggcttgaatg ggtatcgtac atttcgagag ggggaggatc gacgtattac |
| 181 | ccggattccg tgaaaggaag gtttacgatc tcgcgcgaca attcaaagaa tacgctttat |
| 241 | cttcagatga actcgctccg agcggaagat acggcggtat actattgcgg tcgccatgcg |
| 301 | acgacggcgt attggtattt cgatgtgtgg ggacaaggga cgatggtcac ggtgtcgtcg |
| Protein Sequence Defining the Sh4F11 Hv3-23 A28T S31N T62S Heavy Chain | |
| Variable Region | |
| (SEQ ID NO: 40) | |
| 1 | evqllesggg lvqpggslrl scaasgftfs nydmswvrqa pgkglewvsy isrgggstyy |
| 61 | pdsvkgrfti srdnskntly lqmnslraed tavyycgrha ttaywyfdvw gqgtmvtvss |
| Nucleic Acid Sequence Encoding the Sh4F11 Hv3-23 A28T T62S Heavy Chain | |
| Variable Region | |
| (SEQ ID NO: 41) | |
| 1 | gaagtacagt tgttggagtc aggaggaggg ttggtccagc cgggtggatc gttgcggctt |
| 61 | tcgtgtgcgg cgtcgggatt caccttttca tcgtatgaca tgtcgtgggt gaggcaggca |
| 121 | ccggggaaag ggcttgaatg ggtatcgtac atttcgagag ggggaggatc gacgtattac |
| 181 | ccggattccg tgaaaggaag gtttacgatc tcgcgcgaca attcaaagaa tacgctttat |
| 241 | cttcagatga actcgctccg agcggaagat acggcggtat actattgcgg tcgccatgcg |
| 301 | acgacggcgt attggtattt cgatgtgtgg ggacaaggga cgatggtcac ggtgtcgtcg |
| Protein Sequence Defining the Sh4F11 Hv3-23 A28T T62S Heavy Chain | |
| Variable Region | |
| (SEQ ID NO: 42) | |
| 1 | evqllesggg lvqpggslrl scaasgftfs sydmswvrqa pgkglewvsy isrgggstyy |
| 61 | pdsvkgrfti srdnskntly lqmnslraed tavyycgrha ttaywyfdvw gqgtmvtvss |
| Nucleic Acid Sequence Encoding the Ch4F11 Chimeric Kappa Chain Variable | |
| Region | |
| (SEQ ID NO: 32) | |
| 1 | gacgtggtaa tgacgcagac gccgttgtcc cttcctgtct cgctcggaga tcaggcgtcg |
| 61 | atctcgtgta gaagctcgca gtcactcgtc cataccaacg ggaatacata tcttcactgg |
| 121 | tatttgcaaa agcccggaca gtcaccgaag ctcttgatct acaaagtatc caatcggttt |
| 181 | tcgggggtgc ccgaccgatt ctcgggatcg ggttcgggga cggattttac gttgaagatt |
| 241 | tcgcgggtgg aagcggagga tctcggtgtc tacttttgtt cgcagtcaac gcatgtcccg |
| 301 | tggacgttcg gaggcgggac aaaacttgag atcaag |
| Protein Sequence Defining the Ch4F11 Chimeric Kappa Chain Variable | |
| Region | |
| (SEQ ID NO: 4) | |
| 1 | dvvmtqtpls lpvslgdqas iscrssqslv htngntylhw ylqkpgqspk lliykvsnrf |
| 61 | sgvpdrfsgs gsgtdftlki srveaedlgv yfcsqsthvp wtfgggtkle ik |
| Nucleic Acid Sequence Encoding the Hu4F11 Kv2D-29 Kappa Chain Variable | |
| Region | |
| (SEQ ID NO: 43) | |
| 1 | gatgtagtca tgacccaaac gccgctttcg ttgtcggtga cgcccggaca gcccgcgtca |
| 61 | atctcgtgtc ggtcatcgca gtcgttggta cacacaaacg gtaatacgta tctccattgg |
| 121 | tatctccaga agcccggcca gtcgccgcag ctcttgatct acaaagtgag caatcgcttt |
| 181 | tcgggggtgc cggatcggtt ctcgggatcg gggtcaggaa cggacttcac gcttaagatt |
| 241 | tcgagggtcg aggcggagga tgtcggagtc tacttttgtt cgcagtccac acatgtcccc |
| 301 | tggacgtttg ggcaggggac gaaggtggaa atcaag |
| Protein Sequence Defining the Hu4F11 Kv2D-29 Kappa Chain Variable | |
| Region | |
| (SEQ ID NO: 44) | |
| 1 | dvvmtqtpls lsvtpgqpas iscrssqslv htngntylhw ylqkpgqspq lliykvsnrf |
| 61 | sgvpdrfsgs gsgtdftlki srveaedvgv yfcsqsthvp wtfgqgtkve ik |
| Nucleic Acid Sequence Encoding the Hu4F11 Kv2D-29 N28H Kappa Chain | |
| Variable Region | |
| (SEQ ID NO: 45) | |
| 1 | gatgtagtca tgacccaaac gccgctttcg ttgtcggtga cgcccggaca gcccgcgtca |
| 61 | atctcgtgtc ggtcatcgca gtcgttggta cacacacacg gtaatacgta tctccattgg |
| 121 | tatctccaga agcccggcca gtcgccgcag ctcttgatct acaaagtgag caatcgcttt |
| 181 | tcgggggtgc cggatcggtt ctcgggatcg gggtcaggaa cggacttcac gottaagatt |
| 241 | tcgagggtcg aggcggagga tgtcggagtc tacttttgtt cgcagtccac acatgtcccc |
| 301 | tggacgtttg ggcaggggac gaaggtggaa atcaag |
| Protein Sequence Defining the Hu4F11 Kv2D-29 N28H Kappa Chain Variable | |
| Region | |
| (SEQ ID NO: 46) | |
| 1 | dvvmtqtpls lsvtpgqpas iscrssqslv hthgntylhw ylqkpgqspq lliykvsnrf |
| 61 | sgvpdrfsgs gsgtdftlki srveaedvgv yfcsqsthvp wtfgqgtkve ik |
| Nucleic Acid Sequence Encoding the Hu4F11 Kv2D-29 N28Q Kappa Chain | |
| Variable Region | |
| (SEQ ID NO: 47) | |
| 1 | gatgtagtca tgacccaaac gccgctttcg ttgtcggtga cgcccggaca gcccgcgtca |
| 61 | atctcgtgtc ggtcatcgca gtcgttggta cacacacaag gtaatacgta tctccattgg |
| 121 | tatctccaga agcccggcca gtcgccgcag ctcttgatct acaaagtgag caatcgcttt |
| 181 | tcgggggtgc cggatcggtt ctcgggatcg gggtcaggaa cggacttcac gcttaagatt |
| 241 | tcgagggtcg aggcggagga tgtcggagtc tacttttgtt cgcagtccac acatgtcccc |
| 301 | tggacgtttg ggcaggggac gaaggtggaa atcaag |
| Protein Sequence Defining the Hu4F11 Kv2D-29 N28Q Kappa Chain Variable | |
| Region | |
| (SEQ ID NO: 48) | |
| 1 | dvvmtqtpls lsvtpgqpas iscrssqslv htqgntylhw ylqkpgqspq lliykvsnrf |
| 61 | sgvpdrfsgs gsgtdftlki srveaedvgv yfcsqsthvp wtfgqgtkve ik |
| Nucleic Acid Sequence Encoding the Hu4F11 Kv2D-29 N28Y Kappa Chain | |
| Variable Region | |
| (SEQ ID NO: 49) | |
| 1 | gatgtagtca tgacccaaac gccgctttcg ttgtcggtga cgcccggaca gcccgcgtca |
| 61 | atctcgtgtc ggtcatcgca gtcgttggta cacacatacg gtaatacgta tctccattgg |
| 121 | tatctccaga agcccggcca gtcgccgcag ctcttgatct acaaagtgag caatcgcttt |
| 181 | tcgggggtgc cggatcggtt ctcgggatcg gggtcaggaa cggacttcac gcttaagatt |
| 241 | tcgagggtcg aggcggagga tgtcggagtc tacttttgtt cgcagtccac acatgtcccc |
| 301 | tggacgtttg ggcaggggac gaaggtggaa atcaag |
| Protein Sequence Defining the Hu4F11 Kv2D-29 N28Y Kappa Chain Variable | |
| Region | |
| (SEQ ID NO: 50) | |
| 1 | dvvmtqtpls lsvtpgqpas iscrssqslv htygntylhw ylqkpgqspq lliykvsnrf |
| 61 | sgvpdrfsgs gsgtdftlki srveaedvgv yfcsqsthvp wtfgqgtkve ik |

The amino acid sequences defining the immunoglobulin heavy chain variable regions for the antibodies produced in Example 8 are aligned in FIG. 7. Amino terminal signal peptide sequences (for proper expression/secretion) are not shown. CDR₁, CDR₂, and CDR₃ (Kabat definition) are identified by boxes. FIG. 8 show an alignment of the separate CDR₁, CDR₂, and CDR₃ sequences for each of the variable region sequences shown in FIG. 7.

The amino acid sequences defining the immunoglobulin light chain variable regions for the antibodies in Example 8 are aligned in FIG. 9. Amino terminal signal peptide sequences (for proper expression/secretion) are not shown. CDR₁, CDR₂ and CDR₃ are identified by boxes. FIG. 10 shows an alignment of the separate CDR₁, CDR₂, and CDR₃ sequences for each of the variable region sequences shown in FIG. 9.

Table 9 is a concordance chart showing the SEQ ID NO. of each sequence discussed in this Example.

TABLE 9

| SEQ. ID | |
|---|---|
| NO. | Nucleic Acid or Protein |
| 31 | Ch4F11 Chimeric Heavy Chain Variable Region-nucleic acid |
| 2 | Ch4F11 Chimeric Heavy Chain Variable Region-protein |
| 5 | Ch4F11 Chimeric Heavy Chain CDR₁ |
| 6 | Ch4F11 Chimeric Heavy Chain CDR₂ |
| 7 | Ch4F11 Chimeric Heavy Chain CDR₃ |
| 33 | Sh4F11 Hv3-23 Heavy Chain Variable Region-nucleic acid |
| 34 | Sh4F11 Hv3-23 Heavy Chain Variable Region-protein |
| 5 | Sh4F11 Hv3-23 Heavy Chain CDR₁ |
| 6 | Sh4F11 Hv3-23 Heavy Chain CDR₂ |
| 7 | Sh4F11 Hv3-23 Heavy Chain CDR₃ |
| 35 | Sh4F11 Hv3-23 A28T S31H T62S Heavy Chain Variable |
| | Region-nucleic acid |
| 36 | Sh4F11 Hv3-23 A28T S31H T62S Heavy Chain Variable |
| | Region-protein |
| 51 | Sh4F11 Hv3-23 A28T S31H T62S Heavy Chain CDR1 |
| 53 | Sh4F11 Hv3-23 A28T S31H T62S Heavy Chain CDR₂ |
| 7 | Sh4F11 Hv3-23 A28T S31H T62S Heavy Chain CDR₃ |
| 37 | Sh4F11 Hv3-23 S31H T62S Heavy Chain Variable Region- |
| | nucleic acid |
| 38 | Sh4F11 Hv3-23 S31H T62S Heavy Chain Variable Region- |
| | protein |
| 51 | Sh4F11 Hv3-23 S31H T62S Heavy Chain CDR₁ |
| 53 | Sh4F11 Hv3-23 S31H T62S Heavy Chain CDR₂ |
| 7 | Sh4F11 Hv3-23 S31H T62S Heavy Chain CDR₃ |
| 39 | Sh4F11 Hv3-23 A28T S31N T62S Heavy Chain Variable |
| | Region-nucleic acid |
| 40 | Sh4F11 Hv3-23 A28T S31N T62S Heavy Chain Variable |
| | Region-protein |
| 52 | Sh4F11 Hv3-23 A28T S31N T62S Heavy Chain CDR₁ |
| 53 | Sh4F11 Hv3-23 A28T S31N T62S Heavy Chain CDR₂ |
| 7 | Sh4F11 Hv3-23 A28T S31N T62S Heavy Chain CDR₃ |
| 41 | Sh4F11 Hv3-23 A28T T62S Heavy Chain Variable Region- |
| | nucleic acid |
| 42 | Sh4F11 Hv3-23 A28T T62S Heavy Chain Variable Region- |
| | protein |
| 5 | Sh4F11 Hv3-23 A28T T62S Heavy Chain CDR₁ |
| 53 | Sh4F11 Hv3-23 A28T T62S Heavy Chain CDR₂ |
| 7 | Sh4F11 Hv3-23 A28T T62S Heavy Chain CDR₃ |
| 32 | Ch4F11 Chimeric Light (kappa) Chain Variable Region- |
| | nucleic acid |
| 4 | Ch4F11 Chimeric Light (kappa) Chain Variable Region- |
| | protein |
| 8 | Ch4F11 Chimeric Light (kappa) Chain CDR₁ |
| 9 | Ch4F11 Chimeric Light (kappa) Chain CDR₂ |
| 10 | Ch4F11 Chimeric Light (kappa) Chain CDR₃ |
| 43 | Hu4F11 Kv2D-29 Light (kappa) Chain Variable Region- |
| | nucleic acid |
| 44 | Hu4F11 Kv2D-29 Light (kappa) Chain Variable Region- |
| | protein |
| 8 | Hu4F11 Kv2D-29 Light (kappa) Chain CDR₁ |
| 9 | Hu4F11 Kv2D-29 Light (kappa) Chain CDR₂ |
| 10 | Hu4F11 Kv2D-29 Light (kappa) Chain CDR₃ |
| 45 | Hu4F11 Kv2D-29 N28H Light (kappa) Chain Variable |
| | Region-nucleic acid |
| 46 | Hu4F11 Kv2D-29 N28H Light (kappa) Chain Variable |
| | Region-protein |
| 62 | Hu4F11 Kv2D-29 N28H Light (kappa) Chain CDR₁ |
| 9 | Hu4F11 Kv2D-29 N28H Light (kappa) Chain CDR₂ |
| 10 | Hu4F11 Kv2D-29 N28H Light (kappa) Chain CDR₃ |
| 47 | Hu4F11 Kv2D-29 N28Q Light (kappa) Chain Variable |
| | Region-nucleic acid |
| 48 | Hu4F11 Kv2D-29 N28Q Light (kappa) Chain Variable |
| | Region-protein |
| 63 | Hu4F11 Kv2D-29 N28Q Light (kappa) Chain CDR₁ |
| 9 | Hu4F11 Kv2D-29 N28Q Light (kappa) Chain CDR₂ |
| 10 | Hu4F11 Kv2D-29 N28Q Light (kappa) Chain CDR₃ |
| 49 | Hu4F11 Kv2D-29 N28Y Light (kappa) Chain Variable |
| | Region-nucleic acid |
| 50 | Hu4F11 Kv2D-29 N28Y Light (kappa) Chain Variable |
| | Region-protein |
| 64 | Hu4F11 Kv2D-29 N28Y Light (kappa) Chain CDR₁ |
| 9 | Hu4F11 Kv2D-29 N28Y Light (kappa) Chain CDR₂ |
| 10 | Hu4F11 Kv2D-29 N28Y Light (kappa) Chain CDR₃ |

Humanized monoclonal antibody heavy chain CDR sequences (Kabat, Chothia, and IMGT definitions) are shown in Table 10.

TABLE 10

| | | | | |
|---|---|---|---|---|
| | | | | Variable |
| | | | | Region |
| | | | | SEQ ID |
| Kabat | CDR1 | CDR2 | CDR3 | NO: |

Kabat

| | | | | |
|---|---|---|---|---|
| Ch4F11 | SYDMS | YISRGGGSTYYPDTVKG | HATTAYWYFDV | 2 |
| Chimeric | (SEQ ID NO: 5) | (SEQ ID NO: 6) | (SEQ ID NO: 7) | |
| Sh4F11 Hv3- | SYDMS | YISRGGGSTYYPDTVKG | HATTAYWYFDV | 34 |
| 23 | (SEQ ID NO: 5) | (SEQ ID NO: 6) | (SEQ ID NO: 7) | |
| Sh4F11 Hv3- | HYDMS | YISRGGGSTYYPDSVKG | HATTAYWYFDV | 36 |
| 23 A28T | (SEQ ID NO: 51) | (SEQ ID NO: 53) | (SEQ ID NO: 7) | |
| S31H T62S | | | | |
| Sh4F11 Hv3- | HYDMS | YISRGGGSTYYPDSVKG | HATTAYWYFDV | 38 |
| 23 S31H | (SEQ ID NO: 51) | (SEQ ID NO: 53) | (SEQ ID NO: 7) | |
| T62S | | | | |
| Sh4F11 Hv3- | NYDMS | YISRGGGSTYYPDSVKG | HATTAYWYFDV | 40 |
| 23 A28T | (SEQ ID NO: 52) | (SEQ ID NO: 53) | (SEQ ID NO: 7) | |
| S31N T62S | | | | |
| Sh4F11 Hv3- | SYDMS | YISRGGGSTYYPDSVKG | HATTAYWYFDV | 42 |
| 23 A28T | (SEQ ID NO: 5) | (SEQ ID NO: 53) | (SEQ ID NO: 7) | |
| T62S | | | | |

Chothia

| | | | | |
|---|---|---|---|---|
| Ch4F11 | GFAFSSY | SRGGGS | HATTAYWYFDV | 2 |
| Chimeric | (SEQ ID NO: 11) | (SEQ ID NO: 12) | (SEQ ID NO: 7) | |
| Sh4F11 Hv3- | GFAFSSY | SRGGGS | HATTAYWYFDV | 34 |
| 23 | (SEQ ID NO: 11) | (SEQ ID NO: 12) | (SEQ ID NO: 7) | |
| Sh4F11 Hv3- | GFTFSHY | SRGGGS | HATTAYWYFDV | 36 |
| 23 A28T | (SEQ ID NO: 54) | (SEQ ID NO: 12) | (SEQ ID NO: 7) | |
| S31H T62S | | | | |
| Sh4F11 Hv3- | GFAFSHY | SRGGGS | HATTAYWYFDV | 38 |
| 23 S31H | (SEQ ID NO: 55) | (SEQ ID NO: 12) | (SEQ ID NO: 7) | |
| T62S | | | | |
| Sh4F11 Hv3- | GFTFSNY | SRGGGS | HATTAYWYFDV | 40 |
| 23 A28T | (SEQ ID NO: 56) | (SEQ ID NO: 12) | (SEQ ID NO: 7) | |
| S31N T62S | | | | |
| Sh4F11 Hv3- | GFTFSSY | SRGGGS | HATTAYWYFDV | 42 |
| 23 A28T | (SEQ ID NO: 57) | (SEQ ID NO: 12) | (SEQ ID NO: 7) | |
| T62S | | | | |

IMGT

| | | | | |
|---|---|---|---|---|
| Ch4F11 | GFAFSSYD | ISRGGGST | GRHATTAYWYFDV | 2 |
| Chimeric | (SEQ ID NO: 14) | (SEQ ID NO: 15) | (SEQ ID NO: 16) | |
| Sh4F11 Hv3- | GFAFSSYD | ISRGGGST | GRHATTAYWYFDV | 34 |
| 23 | (SEQ ID NO: 14) | (SEQ ID NO: 15) | (SEQ ID NO: 16) | |
| Sh4F11 Hv3- | GFTFSHYD | ISRGGGST | GRHATTAYWYFDV | 36 |
| 23 A28T | (SEQ ID NO: 58) | (SEQ ID NO: 15) | (SEQ ID NO: 16) | |
| S31H T62S | | | | |
| Sh4F11 Hv3- | GFAFSHYD | ISRGGGST | GRHATTAYWYFDV | 38 |
| 23 S31H | (SEQ ID NO: 59) | (SEQ ID NO: 15) | (SEQ ID NO: 16) | |
| T62S | | | | |
| Sh4F11 Hv3- | GFTFSNYD | ISRGGGST | GRHATTAYWYFDV | 40 |
| 23 A28T | (SEQ ID NO: 60) | (SEQ ID NO: 15) | (SEQ ID NO: 16) | |
| S31N T62S | | | | |
| Sh4F11 Hv3- | GFTFSSYD | ISRGGGST | GRHATTAYWYFDV | 42 |
| 23 A28T | (SEQ ID NO: 61) | (SEQ ID NO: 15) | (SEQ ID NO: 16) | |
| T62S | | | | |

Humanized monoclonal antibody Kappa light chain CDR sequences (Kabat, Chothia, and IMGT definitions) are shown in Table 11.

TABLE 11

| | | | |
|---|---|---|---|
| | | | Variable |
| | | | Region |
| CDR1 | CDR2 | CDR3 | SEQ ID NO: |

Kabat/Chothia

| | | | | |
|---|---|---|---|---|
| Ch4F11 | RSSQSL VHTNGNTYLH | KVSNRFS | SQSTHVPWT | 4 |
| Chimeric | (SEQ ID NO: 8) | (SEQ ID NO: 9) | (SEQ ID NO: 10) | |
| Hu4F11 | RSSQSL VHTNGNTYLH | KVSNRFS | SQSTHVPWT | 44 |
| Kv2D-29 | (SEQ ID NO: 8) | (SEQ ID NO: 9) | (SEQ ID NO: 10) | |
| Hu4F11 | RSSQSL VHTHGNTYLH | KVSNRFS | SQSTHVPWT | 46 |
| Kv2D-29 | (SEQ ID NO: 62) | (SEQ ID NO: 9) | (SEQ ID NO: 10) | |
| N28H | | | | |
| Hu4F11 | RSSQSL VHTQGNTYLH | KVSNRFS | SQSTHVPWT | 48 |
| Kv2D-29 | (SEQ ID NO: 63) | (SEQ ID NO: 9) | (SEQ ID NO: 10) | |
| N28Q | | | | |
| Hu4F11 | RSSQSL VHTYGNTYLH | KVSNRFS | SQSTHVPWT | 50 |
| Kv2D-29 | (SEQ ID NO: 64) | (SEQ ID NO: 9) | (SEQ ID NO: 10) | |
| N28Y | | | | |

IMGT

| | | | | |
|---|---|---|---|---|
| Ch4F11 | QSL VHTNGNTY | KVS | SQSTHVPWT | 4 |
| Chimeric | (SEQ ID NO: 20) | | (SEQ ID NO: 10) | |
| Hu4F11 | QSL VHTNGNTY | KVS | SQSTHVPWT | 44 |
| Kv2D-29 | (SEQ ID NO: 20) | | (SEQ ID NO: 10) | |
| Hu4F11 | QSL VHTHGNTY | KVS | SQSTHVPWT | 46 |
| Kv2D-29 | (SEQ ID NO: 65) | | (SEQ ID NO: 10) | |
| N28H | | | | |
| Hu4F11 | QSL VHTQGNTY | KVS | SQSTHVPWT | 48 |
| Kv2D-29 | (SEQ ID NO: 66) | | (SEQ ID NO: 10) | |
| N28Q | | | | |
| Hu4F11 | QSL VHTYGNTY | KVS | SQSTHVPWT | 50 |
| Kv2D-29 | (SEQ ID NO: 67) | | (SEQ ID NO: 10) | |
| N28Y | | | | |

To create the complete chimeric and humanized heavy or kappa chain antibody sequences, each variable sequence above is combined with its respective human constant region. For example, a complete heavy chain comprises a heavy variable sequence followed by a human IgG1 heavy chain constant sequence. A complete kappa chain comprises a kappa variable sequence followed by the human kappa light chain constant sequence.

| Nucleic Acid Sequence Encoding the Human IgG1 Heavy Chain Constant | |
|---|---|
| Region_ | |
| (SEQ ID NO: 68) | |
| 1 | gcctcaacaa aaggaccaag tgtgttccca ctcgccccta gcagcaagag tacatccggg |
| 61 | ggcactgcag cactcggctg cctcgtcaag gattattttc cagagccagt aaccgtgagc |
| 121 | tggaacagtg gagcactcac ttctggtgtc catacttttc ctgctgtcct gcaaagctct |
| 181 | ggcctgtact cactcagctc cgtcgtgacc gtgccatctt catctctggg cactcagacc |
| 241 | tacatctgta atgtaaacca caagcctagc aatactaagg tcgataagcg ggtggaaccc |
| 301 | aagagctgcg acaagactca cacttgtccc ccatgccctg cccctgaact tctgggcggt |
| 361 | cccagcgtct ttttgttccc accaaagcct aaagatactc tgatgataag tagaacaccc |
| 421 | gaggtgacat gtgttgttgt agacgtttcc cacgaggacc cagaggttaa gttcaactgg |
| 481 | tacgttgatg gagtcgaagt acataatgct aagaccaagc ctagagagga gcagtataat |
| 541 | agtacatacc gtgtagtcag tgttctcaca gtgctgcacc aagactggct caacggcaaa |
| 601 | gaatacaaat gcaaagtgtc caacaaagca ctcccagccc ctatcgagaa gactattagt |
| 661 | aaggcaaagg ggcagcctcg tgaaccacag gtgtacactc tgccacccag tagagaggaa |
| 721 | atgacaaaga accaagtctc attgacctgc ctggtgaaag gcttctaccc cagcgacatc |
| 781 | gccgttgagt gggagagtaa cggtcagcct gagaacaatt acaagacaac ccccccagtg |
| 841 | ctggatagtg acgggtcttt ctttctgtac agtaagctga ctgtggacaa gtcccgctgg |
| 901 | cagcagggta acgtcttcag ctgttccgtg atgcacgagg cattgcacaa ccactacacc |
| 961 | cagaagtcac tgagcctgag cccagggaag |
| Protein Sequence Defining the Human IgG1 Heavy Chain Constant Region | |
| (SEQ ID NO: 69) | |
| 1 | astkgpsvfp lapsskstsg gtaalgclvk dyfpepvtvs wnsgaltsgv htfpavlqss |
| 61 | glyslssvvt vpssslgtqt yicnvnhkps ntkvdkrvep kscdkthtcp pcpapellgg |
| 121 | psvflfppkp kdtlmisrtp evtcvvvdvs hedpevkfnw yvdgvevhna ktkpreeqyn |
| 181 | styrvvsvlt vlhqdwlngk eykckvsnka lpapiektis kakgqprepq vytlppsree |
| 241 | mtknqvsltc lvkgfypsdi avewesngqp ennykttppv ldsdgsffly skltvdksrw |
| 301 | qqgnvfscsv mhealhnhyt qkslslspgk |
| Nucleic Acid Sequence Encoding the Human Kappa Light Chain Constant | |
| Region (Version 1) | |
| (SEQ ID NO: 70) | |
| 1 | cgcacagttg ctgcccccag cgtgttcatt ttcccaccta gcgatgagca gctgaaaagc |
| 61 | ggtactgcct ctgtcgtatg cttgctcaac aacttttacc cacgtgaggc taaggtgcag |
| 121 | tggaaagtgg ataatgcact tcaatctgga aacagtcaag agtccgtgac agaacaggac |
| 181 | agcaaagact caacttattc actctcttcc accctgactc tgtccaaggc agactatgaa |
| 241 | aaacacaagg tatacgcctg cgaggttaca caccagggtt tgtctagtcc tgtcaccaag |
| 301 | tccttcaata ggggcgaatg t |
| Nucleic Acid Sequence Encoding the Human Kappa Light Chain Constant | |
| Region (Version 2)_ | |
| (SEQ ID NO: 71) | |
| 1 | cgcacagttg cagcccccag cgtgttcatt ttcccaccta gcgatgagca gctgaaaagc |
| 61 | ggtactgcct ctgtcgtatg cttgctcaac aacttttacc cacgtgaggc taaggtgcag |
| 121 | tggaaagtgg ataatgcact tcaatctgga aacagtcaag agtccgtgac agaacaggac |
| 181 | agcaaagact caacttattc actctcttcc accctgactc tgtccaaggc agactatgaa |
| 241 | aaacacaagg tatacgcctg cgaggttaca caccagggtt tgtctagtcc tgtcaccaag |
| 301 | tccttcaata ggggcgaatg t |
| Protein Sequence Defining the Human Kappa Light Chain Constant Region | |
| (nucleic acid versions 1 and 2 encode the same amino sequence) | |
| (SEQ ID NO: 72) | |
| 1 | rtvaapsvfi fppsdeqlks gtasvvclln nfypreakvq wkvdnalqsg nsqesvteqd |
| 61 | skdstyslss tltlskadye khkvyacevt hqglsspvtk sfnrgec |

The following sequences represent the actual or contemplated full length heavy and light chain sequence (i.e., containing both the variable and constant regions sequences) for each antibody described in this Example. Signal sequences for proper secretion of the antibodies (e.g., signal sequences at the 5′ end of the DNA sequences or the amino terminal end of the protein sequences) are not shown in the full length heavy and light chain sequences disclosed herein and are not included in the final secreted protein. Also not shown are stop codons for termination of translation required at the 3′ end of the DNA sequences. It is within ordinary skill in the art to select a signal sequence and/or a stop codon for expression of the disclosed full length immunoglobulin heavy chain and light chain sequences. It is also contemplated that the variable region sequences can be ligated to other constant region sequences to produce active full length immunoglobulin heavy and light chains.

| Nucleic Acid Sequence Encoding the Full Length Ch4F11 Chimeric Heavy | |
|---|---|
| Chain (Mouse Heavy Chain Variable Region and Human IgG1 Constant Region) | |
| (SEQ ID NO: 73) | |
| 1 | gaggtacagc ttgtcgagtc gggaggagga ttggtaaaac cgggtgggtc actcaaattg |
| 61 | tcgtgtgcgg cgtcgggatt tgcgttttcg tcgtatgata tgtcgtgggt gcgccagacg |
| 121 | ccggaaaaac gattggaatg ggtcgcgtat atctcccgag ggggaggttc gacatactat |
| 181 | cccgacacgg tcaaagggcg cttcacgatt tcacgggaca atgcgaaaaa cacgctttat |
| 241 | cttcaaatgt cgtcgttgaa atcggaagat accgcgatgt attactgcgg gaggcatgcg |
| 301 | acgacggcgt attggtattt cgatgtgtgg ggagccggaa cgacggtgac ggtgtcgtcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |
| Protein Sequence Defining the Full Length Ch4F11 Chimeric Heavy Chain | |
| (Mouse Heavy Chain Variable Region and Human IgG1 Constant Region) | |
| (SEQ ID NO: 74) | |
| 1 | evqlvesggg lvkpggslkl scaasgfafs sydmswvrqt pekrlewvay isrgggstyy |
| 61 | pdtvkgrfti srdnakntly lqmsslksed tamyycgrha ttaywyfdvw gagttvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| Nucleic Acid Sequence Encoding the Full Length Sh4F11 Hv3-23 Heavy Chain | |
| (Humanized Heavy Chain Variable Region and Human IgG1 Constant Region) | |
| (SEQ ID NO: 75) | |
| 1 | gaagtacagt tgttggagtc aggaggaggg ttggtccagc cgggtggatc gttgcggctt |
| 61 | tcgtgtgcgg cgtcgggatt cgcgttttca tcgtatgaca tgtcgtgggt gaggcaggca |
| 121 | ccggggaaag ggcttgaatg ggtatcgtac atttcgagag ggggaggatc gacgtattac |
| 181 | ccggatacgg tgaaaggaag gtttacgatc tcgcgcgaca attcaaagaa tacgctttat |
| 241 | cttcagatga actcgctccg agcggaagat acggcggtat actattgcgg tcgccatgcg |
| 301 | acgacggcgt attggtattt cgatgtgtgg ggacaaggga cgatggtcac ggtgtcgtcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |
| Protein Sequence Defining the Full Length Sh4F11 Hv3-23 Heavy Chain | |
| (Humanized Heavy Chain Variable Region and Human IgG1 Constant Region) | |
| (SEQ ID NO: 76) | |
| 1 | evqllesggg lvqpggslrl scaasgfafs sydmswvrqa pgkglewvsy isrgggstyy |
| 61 | pdtvkgrfti srdnskntly lqmnslraed tavyycgrha ttaywyfdvw gqgtmvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| Nucleic Acid Sequence Encoding the Full Length Sh4F11 Hv3-23 A28T S31H | |
| T62S Heavy Chain (Humanized Heavy Chain Variable Region and Human IgG1 | |
| Constant Region) | |
| (SEQ ID NO: 77) | |
| 1 | gaagtacagt tgttggagtc aggaggaggg ttggtccagc cgggtggatc gttgcggctt |
| 61 | tcgtgtgcgg cgtcgggatt caccttttca cactatgaca tgtcgtgggt gaggcaggca |
| 121 | ccggggaaag ggcttgaatg ggtatcgtac atttcgagag ggggaggatc gacgtattac |
| 181 | ccggattccg tgaaaggaag gtttacgatc tcgcgcgaca attcaaagaa tacgctttat |
| 241 | cttcagatga actcgctccg agcggaagat acggcggtat actattgcgg tcgccatgcg |
| 301 | acgacggcgt attggtattt cgatgtgtgg ggacaaggga cgatggtcac ggtgtcgtcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |
| Protein Sequence Defining the Full Length Sh4F11 Hv3-23 A28T S31H T62S | |
| Heavy Chain (Humanized Heavy Chain Variable Region and Human IgG1 Constant | |
| Region) | |
| (SEQ ID NO: 78) | |
| 1 | evqllesggg lvqpggslrl scaasgftfs hydmswvrqa pgkglewvsy isrgggstyy |
| 61 | pdsvkgrfti srdnskntly lqmnslraed tavyycgrha ttaywyfdvw gqgtmvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| Nucleic Acid Sequence Encoding the Full Length Sh4F11 Hv3-23 S31H T62S | |
| Heavy Chain (Humanized Heavy Chain Variable Region and Human IgG1 Constant | |
| Region) | |
| (SEQ ID NO: 79) | |
| 1 | gaagtacagt tgttggagtc aggaggaggg ttggtccagc cgggtggatc gttgcggctt |
| 61 | tcgtgtgcgg cgtcgggatt cgcgttttca cactatgaca tgtcgtgggt gaggcaggca |
| 121 | ccggggaaag ggcttgaatg ggtatcgtac atttcgagag ggggaggatc gacgtattac |
| 181 | ccggattccg tgaaaggaag gtttacgatc tcgcgcgaca attcaaagaa tacgctttat |
| 241 | cttcagatga actcgctccg agcggaagat acggcggtat actattgcgg tcgccatgcg |
| 301 | acgacggcgt attggtattt cgatgtgtgg ggacaaggga cgatggtcac ggtgtcgtcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |
| Protein Sequence Defining the Full Length Sh4F11 Hv3-23 S31H T62S Heavy | |
| Chain (Humanized Heavy Chain Variable Region and Human IgG1 Constant Region) | |
| (SEQ ID NO: 80) | |
| 1 | evqllesggg lvqpggslrl scaasgfafs hydmswvrqa pgkglewvsy isrgggstyy |
| 61 | pdsvkgrfti srdnskntly lqmnslraed tavyycgrha ttaywyfdvw gqgtmvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| Nucleic Acid Sequence Encoding the Full Length Sh4F11 Hv3-23 A28T S31N | |
| T62S Heavy Chain (Humanized Heavy Chain Variable Region and Human IgG1 | |
| Constant Region)_ | |
| (SEQ ID NO: 81) | |
| 1 | gaagtacagt tgttggagtc aggaggaggg ttggtccagc cgggtggatc gttgcggctt |
| 61 | tcgtgtgcgg cgtcgggatt caccttttca aactatgaca tgtcgtgggt gaggcaggca |
| 121 | ccggggaaag ggcttgaatg ggtatcgtac atttcgagag ggggaggatc gacgtattac |
| 181 | ccggattccg tgaaaggaag gtttacgatc tcgcgcgaca attcaaagaa tacgctttat |
| 241 | cttcagatga actcgctccg agcggaagat acggcggtat actattgcgg tcgccatgcg |
| 301 | acgacggcgt attggtattt cgatgtgtgg ggacaaggga cgatggtcac ggtgtcgtcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |
| Protein Sequence Defining the Full Length Sh4F11 A28T S31N T62S Heavy | |
| Chain (Humanized Heavy Chain Variable Region and Human IgG1 Constant Region) | |
| (SEQ ID NO: 82) | |
| 1 | evqllesggg lvqpggslrl scaasgfafs hydmswvrqa pgkglewvsy isrgggstyy |
| 61 | pdsvkgrfti srdnskntly lqmnslraed tavyycgrha ttaywyfdvw gqgtmvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| Nucleic Acid Sequence Encoding the Full Length Sh4F11 Hv3-23 A28T T62S | |
| Heavy Chain (Humanized Heavy Chain Variable Region and Human IgG1 Constant | |
| Region) | |
| (SEQ ID NO: 83) | |
| 1 | gaagtacagt tgttggagtc aggaggaggg ttggtccagc cgggtggatc gttgcggctt |
| 61 | tcgtgtgcgg cgtcgggatt caccttttca tcgtatgaca tgtcgtgggt gaggcaggca |
| 121 | ccggggaaag ggcttgaatg ggtatcgtac atttcgagag ggggaggatc gacgtattac |
| 181 | ccggattccg tgaaaggaag gtttacgatc tcgcgcgaca attcaaagaa tacgctttat |
| 241 | cttcagatga actcgctccg agcggaagat acggcggtat actattgcgg tcgccatgcg |
| 301 | acgacggcgt attggtattt cgatgtgtgg ggacaaggga cgatggtcac ggtgtcgtcg |
| 361 | gcctcaacaa aaggaccaag tgtgttccca ctcgccccta gcagcaagag tacatccggg |
| 421 | ggcactgcag cactcggctg cctcgtcaag gattattttc cagagccagt aaccgtgagc |
| 481 | tggaacagtg gagcactcac ttctggtgtc catacttttc ctgctgtcct gcaaagctct |
| 541 | ggcctgtact cactcagctc cgtcgtgacc gtgccatctt catctctggg cactcagacc |
| 601 | tacatctgta atgtaaacca caagcctagc aatactaagg tcgataagcg ggtggaaccc |
| 661 | aagagctgcg acaagactca cacttgtccc ccatgccctg cccctgaact tctgggcggt |
| 721 | cccagcgtct ttttgttccc accaaagcct aaagatactc tgatgataag tagaacaccc |
| 781 | gaggtgacat gtgttgttgt agacgtttcc cacgaggacc cagaggttaa gttcaactgg |
| 841 | tacgttgatg gagtcgaagt acataatgct aagaccaagc ctagagagga gcagtataat |
| 901 | agtacatacc gtgtagtcag tgttctcaca gtgctgcacc aagactggct caacggcaaa |
| 961 | gaatacaaat gcaaagtgtc caacaaagca ctcccagccc ctatcgagaa gactattagt |
| 1021 | aaggcaaagg ggcagcctcg tgaaccacag gtgtacactc tgccacccag tagagaggaa |
| 1081 | atgacaaaga accaagtctc attgacctgc ctggtgaaag gcttctaccc cagcgacatc |
| 1141 | gccgttgagt gggagagtaa cggtcagcct gagaacaatt acaagacaac ccccccagtg |
| 1201 | ctggatagtg acgggtcttt ctttctgtac agtaagctga ctgtggacaa gtcccgctgg |
| 1261 | cagcagggta acgtcttcag ctgttccgtg atgcacgagg cattgcacaa ccactacacc |
| 1321 | cagaagtcac tgagcctgag cccagggaag |
| Protein Sequence Defining the Full Length Sh4F11 Hv3-23 A28T T62SHeavy | |
| Chain (Humanized Heavy Chain Variable Region and Human IgG1 Constant Region) | |
| (SEQ ID NO: 84) | |
| 1 | evqllesggg lvqpggslrl scaasgftfs sydmswvrqa pgkglewvsy isrgggstyy |
| 61 | pdsvkgrfti srdnskntly lqmnslraed tavyycgrha ttaywyfdvw gqgtmvtvss |
| 121 | astkgpsvfp lapsskstsg gtaalgclvk dyfpepvtvs wnsgaltsgv htfpavlqss |
| 181 | glyslssvvt vpssslgtqt yicnvnhkps ntkvdkrvep kscdkthtcp pcpapellgg |
| 241 | psvflfppkp kdtlmisrtp evtcvvvdvs hedpevkfnw yvdgvevhna ktkpreeqyn |
| 301 | styrvvsvlt vlhqdwlngk eykckvsnka lpapiektis kakgqprepq vytlppsree |
| 361 | mtknqvsltc lvkgfypsdi avewesngqp ennykttppv ldsdgsffly skltvdksrw |
| 421 | qqgnvfscsv mhealhnhyt qkslslspgk |
| Nucleic Acid Sequence Encoding the Full Length Ch4F11 Chimeric Light | |
| Chain (Mouse Kappa Chain Variable Region and Human Kappa Constant Region) | |
| (SEQ ID NO: 85) | |
| 1 | gacgtggtaa tgacgcagac gccgttgtcc cttcctgtct cgctcggaga tcaggcgtcg |
| 61 | atctcgtgta gaagctcgca gtcactcgtc cataccaacg ggaatacata tcttcactgg |
| 121 | tatttgcaaa agcccggaca gtcaccgaag ctcttgatct acaaagtatc caatcggttt |
| 181 | tcgggggtgc ccgaccgatt ctcgggatcg ggttcgggga cggattttac gttgaagatt |
| 241 | tcgcgggtgg aagcggagga tctcggtgtc tacttttgtt cgcagtcaac gcatgtcccg |
| 301 | tggacgttcg gaggcgggac aaaacttgag atcaagcgca cagttgctgc ccccagcgtg |
| 361 | ttcattttcc cacctagcga tgagcagctg aaaagcggta ctgcctctgt cgtatgcttg |
| 421 | ctcaacaact tttacccacg tgaggctaag gtgcagtgga aagtggataa tgcacttcaa |
| 481 | tctggaaaca gtcaagagtc cgtgacagaa caggacagca aagactcaac ttattcactc |
| 541 | tcttccaccc tgactctgtc caaggcagac tatgaaaaac acaaggtata cgcctgcgag |
| 601 | gttacacacc agggtttgtc tagtcctgtc accaagtcct tcaatagggg cgaatgt |
| Protein Sequence Defining the Full Length Ch4F11 Chimeric Light Chain | |
| (Mouse Kappa Chain Variable Region and Human Kappa Constant Region) | |
| (SEQ ID NO: 86) | |
| 1 | dvvmtqtpls lpvslgdqas iscrssqslv htngntylhw ylqkpgqspk lliykvsnrf |
| 61 | sgvpdrfsgs gsgtdftlki srveaedlgv yfcsqsthvp wtfgggtkle ikrtvaapsv |
| 121 | fifppsdeql ksgtasvvcl lnnfypreak vqwkvdnalq sgnsqesvte qdskdstysl |
| 181 | sstltlskad yekhkvyace vthqglsspv tksfnrgec |
| Nucleic Acid Sequence Encoding the Full Length Hu4F11 Kv2D-29 Light | |
| Chain (Humanized Kappa Chain Variable Region and Human Kappa Constant Region | |
| (Version 1)) | |
| (SEQ ID NO: 87) | |
| 1 | gatgtagtca tgacccaaac gccgctttcg ttgtcggtga cgcccggaca gcccgcgtca |
| 61 | atctcgtgtc ggtcatcgca gtcgttggta cacacaaacg gtaatacgta tctccattgg |
| 121 | tatctccaga agcccggcca gtcgccgcag ctcttgatct acaaagtgag caatcgcttt |
| 181 | tcgggggtgc cggatcggtt ctcgggatcg gggtcaggaa cggacttcac gcttaagatt |
| 241 | tcgagggtcg aggcggagga tgtcggagtc tacttttgtt cgcagtccac acatgtcccc |
| 301 | tggacgtttg ggcaggggac gaaggtggaa atcaagcgca cagttgctgc ccccagcgtg |
| 361 | ttcattttcc cacctagcga tgagcagctg aaaagcggta ctgcctctgt cgtatgcttg |
| 421 | ctcaacaact tttacccacg tgaggctaag gtgcagtgga aagtggataa tgcacttcaa |
| 481 | tctggaaaca gtcaagagtc cgtgacagaa caggacagca aagactcaac ttattcactc |
| 541 | tcttccaccc tgactctgtc caaggcagac tatgaaaaac acaaggtata cgcctgcgag |
| 601 | gttacacacc agggtttgtc tagtcctgtc accaagtcct tcaatagggg cgaatgt |
| Protein Sequence Defining the Full Length Hu4F11 Kv2D-29 Light Chain | |
| (Humanized Kappa Chain Variable Region and Human Kappa Constant Region) | |
| (SEQ ID NO: 88) | |
| 1 | dvvmtqtpls lsvtpgqpas iscrssqslv htngntylhw ylqkpgqspq lliykvsnrf |
| 61 | sgvpdrfsgs gsgtdftlki srveaedvgv yfcsqsthvp wtfgqgtkve ikrtvaapsv |
| 121 | fifppsdeql ksgtasvvcl Innfypreak vqwkvdnalq sgnsqesvte qdskdstysl |
| 181 | sstltlskad yekhkvyace vthqglsspv tksfnrgec |
| Nucleic Acid Sequence Encoding the Full Length Hu4F11 Kv2D-29 N28H | |
| Light Chain (Humanized Kappa Chain Variable Region and Human Kappa Constant | |
| Region (Version 2))_ | |
| (SEQ ID NO: 89) | |
| 1 | gatgtagtca tgacccaaac gccgctttcg ttgtcggtga cgcccggaca gcccgcgtca |
| 61 | atctcgtgtc ggtcatcgca gtcgttggta cacacacacg gtaatacgta tctccattgg |
| 121 | tatctccaga agcccggcca gtcgccgcag ctcttgatct acaaagtgag caatcgcttt |
| 181 | tcgggggtgc cggatcggtt ctcgggatcg gggtcaggaa cggacttcac gcttaagatt |
| 241 | tcgagggtcg aggcggagga tgtcggagtc tacttttgtt cgcagtccac acatgtcccc |
| 301 | tggacgtttg ggcaggggac gaaggtggaa atcaagcgca cagttgcagc ccccagcgtg |
| 361 | ttcattttcc cacctagcga tgagcagctg aaaagcggta ctgcctctgt cgtatgcttg |
| 421 | ctcaacaact tttacccacg tgaggctaag gtgcagtgga aagtggataa tgcacttcaa |
| 481 | tctggaaaca gtcaagagtc cgtgacagaa caggacagca aagactcaac ttattcactc |
| 541 | tcttccaccc tgactctgtc caaggcagac tatgaaaaac acaaggtata cgcctgcgag |
| 601 | gttacacacc agggtttgtc tagtcctgtc accaagtcct tcaatagggg cgaatgt |
| Protein Sequence Defining the Full Length Hu4F11 Kv2D-29 N28H Light | |
| Chain (Humanized Kappa Chain Variable Region and Human Kappa Constant Region) | |
| (SEQ ID NO: 90) | |
| 1 | dvvmtqtpls lsvtpgqpas iscrssqslv hthgntylhw ylqkpgqspq lliykvsnrf |
| 61 | sgvpdrfsgs gsgtdftlki srveaedvgv yfcsqsthvp wtfgqgtkve ikrtvaapsv |
| 121 | fifppsdeql ksgtasvvcl Innfypreak vqwkvdnalq sgnsqesvte qdskdstysl |
| 181 | sstltlskad yekhkvyace vthqglsspv tksfnrgec |
| Nucleic Acid Sequence Encoding the Full Length Hu4F11 Kv2D-29 N28Q | |
| Light Chain (Humanized Kappa Chain Variable Region and Human Kappa Constant | |
| Region (Version 2)) | |
| (SEQ ID NO: 91) | |
| 1 | gatgtagtca tgacccaaac gccgctttcg ttgtcggtga cgcccggaca gcccgcgtca |
| 61 | atctcgtgtc ggtcatcgca gtcgttggta cacacacaag gtaatacgta tctccattgg |
| 121 | tatctccaga agcccggcca gtcgccgcag ctcttgatct acaaagtgag caatcgcttt |
| 181 | tcgggggtgc cggatcggtt ctcgggatcg gggtcaggaa cggacttcac gcttaagatt |
| 241 | tcgagggtcg aggcggagga tgtcggagtc tacttttgtt cgcagtccac acatgtcccc |
| 301 | tggacgtttg ggcaggggac gaaggtggaa atcaagcgca cagttgcagc ccccagcgtg |
| 361 | ttcattttcc cacctagcga tgagcagctg aaaagcggta ctgcctctgt cgtatgcttg |
| 421 | ctcaacaact tttacccacg tgaggctaag gtgcagtgga aagtggataa tgcacttcaa |
| 481 | tctggaaaca gtcaagagtc cgtgacagaa caggacagca aagactcaac ttattcactc |
| 541 | tcttccaccc tgactctgtc caaggcagac tatgaaaaac acaaggtata cgcctgcgag |
| 601 | gttacacacc agggtttgtc tagtcctgtc accaagtcct tcaatagggg cgaatgt |
| Protein Sequence Defining the Full Length Hu4F11 Kv2D-29 N28Q Light | |
| Chain (Humanized Kappa Chain Variable Region and Human Kappa Constant Region) | |
| (SEQ ID NO: 92) | |
| 1 | dvvmtqtpls lsvtpgqpas iscrssqslv htqgntylhw ylqkpgqspq lliykvsnrf |
| 61 | sgvpdrfsgs gsgtdftlki srveaedvgv yfcsqsthvp wtfgqgtkve ikrtvaapsv |
| 121 | fifppsdeql ksgtasvvcl Innfypreak vqwkvdnalq sgnsqesvte qdskdstysl |
| 181 | sstltlskad yekhkvyace vthqglsspv tksfnrgec |
| Nucleic Acid Sequence Encoding the Full Length Hu4F11 Kv2D-29 N28Y Light | |
| Chain (Humanized Kappa Chain Variable Region and Human Kappa Constant Region | |
| (Version 2)) | |
| (SEQ ID NO: 93) | |
| 1 | gatgtagtca tgacccaaac gccgctttcg ttgtcggtga cgcccggaca gcccgcgtca |
| 61 | atctcgtgtc ggtcatcgca gtcgttggta cacacatacg gtaatacgta tctccattgg |
| 121 | tatctccaga agcccggcca gtcgccgcag ctcttgatct acaaagtgag caatcgcttt |
| 181 | tcgggggtgc cggatcggtt ctcgggatcg gggtcaggaa cggacttcac gcttaagatt |
| 241 | tcgagggtcg aggcggagga tgtcggagtc tacttttgtt cgcagtccac acatgtcccc |
| 301 | tggacgtttg ggcaggggac gaaggtggaa atcaagcgca cagttgcagc ccccagcgtg |
| 361 | ttcattttcc cacctagcga tgagcagctg aaaagcggta ctgcctctgt cgtatgcttg |
| 421 | ctcaacaact tttacccacg tgaggctaag gtgcagtgga aagtggataa tgcacttcaa |
| 481 | tctggaaaca gtcaagagtc cgtgacagaa caggacagca aagactcaac ttattcactc |
| 541 | tcttccaccc tgactctgtc caaggcagac tatgaaaaac acaaggtata cgcctgcgag |
| 601 | gttacacacc agggtttgtc tagtcctgtc accaagtcct tcaatagggg cgaatgt |
| Protein Sequence Defining the Full Length Hu4F11 Kv2D-29 N28Y Light | |
| Chain (Humanized Kappa Chain Variable Region and Human Kappa Constant Region) | |
| (SEQ ID NO: 94) | |
| 1 | dvvmtqtpls lsvtpgqpas iscrssqslv htygntylhw ylqkpgqspq lliykvsnrf |
| 61 | sgvpdrfsgs gsgtdftlki srveaedvgv yfcsqsthvp wtfgqgtkve ikrtvaapsv |
| 121 | fifppsdeql ksgtasvvcl Innfypreak vqwkvdnalq sgnsqesvte qdskdstysl |
| 181 | sstltlskad yekhkvyace vthqglsspv tksfnrgec |

Table 12 is a concordance chart showing the SEQ ID NO. of each sequence discussed in this Example.

TABLE 12

| SEQ ID | |
|---|---|
| NO. | Nucleic Acid or Protein |
| 68 | Human IgG1 constant-nucleic acid |
| 69 | Human IgG1 constant-protein |
| 70 | Human Kappa constant-nucleic acid (version 1) |
| 71 | Human Kappa constant-nucleic acid (version 2) |
| 72 | Human Kappa constant-protein (nucleic acid versions |
| | 1 and 2 encode the same amino acid sequence) |
| 73 | Chimeric Ch4F11 Heavy Variable + Human IgG1 |
| | constant-nucleic acid |
| 74 | Chimeric Ch4F11 Heavy Variable + Human IgG1 |
| | constant-protein |
| 75 | Humanized Sh4F11 Hv3-23 Heavy Human Variable + |
| | Human IgG1 constant-nucleic acid |
| 76 | Humanized Sh4F11 Hv3-23 Heavy Human Variable + |
| | Human IgG1 constant-protein |
| 77 | Humanized Sh4F11 Hv3-23 A28T S31H T62S Heavy Human |
| | Variable + Human IgG1 constant-nucleic acid |
| 78 | Humanized Sh4F11 Hv3-23 A28T S31H T62S Heavy Human |
| | Variable + Human IgG1 constant-protein |
| 79 | Humanized Sh4F11 Hv3-23 S31H T62S Heavy Human |
| | Variable + Human IgG1 constant-nucleic acid |
| 80 | Humanized Sh4F11 Hv3-23 S31H T62S Heavy Human |
| | Variable + Human IgG1 constant-protein |
| 81 | Humanized Sh4F11 Hv3-23 A28T S31N T62S Heavy Human |
| | Variable + Human IgG1 constant-nucleic acid |
| 82 | Humanized Sh4F11 Hv3-23 A28T S31N T62S Heavy Human |
| | Variable + Human IgG1 constant-protein |
| 83 | Humanized Sh4F11 Hv3-23 A28T T62S Heavy Human |
| | Variable + Human IgG1 constant-nucleic acid |
| 84 | Humanized Sh4F11 Hv3-23 A28T T62S Heavy Human |
| | Variable + Human IgG1 constant-protein |
| 85 | Chimeric Ch4F11 Kappa Variable + Human Kappa constant- |
| | nucleic acid (version 1) |
| 86 | Chimeric Ch4F11 Kappa Variable + Human Kappa constant- |
| | protein |
| 87 | Humanized Hu4F11 Kv2D-29 Kappa Human Variable + Human |
| | Kappa constant-nucleic acid (version 1) |
| 88 | Humanized Hu4F11 Kv2D-29 Kappa Human Variable + Human |
| | Kappa constant-protein |
| 89 | Humanized Hu4F11 Kv2D-29 N28H Kappa Human Variable + |
| | Human Kappa constant-nucleic acid (version 2) |
| 90 | Humanized Hu4F11 Kv2D-29 N28H Kappa Human Variable + |
| | Human Kappa constant-protein |
| 91 | Humanized Hu4F11 Kv2D-29 N28Q Kappa Human Variable + |
| | Human Kappa constant-nucleic acid (version 2) |
| 92 | Humanized Hu4F11 Kv2D-29 N28Q Kappa Human Variable + |
| | Human Kappa constant-protein |
| 93 | Humanized Hu4F11 Kv2D-29 N28Y Kappa Human Variable + |
| | Human Kappa constant-nucleic acid (version 2) |
| 94 | Humanized Hu4F11 Kv2D-29 N28Y Kappa Human Variable + |
| | Human Kappa constant-protein |

Table 13 below shows antibodies containing chimeric immunoglobulin heavy and light chains and exemplary combinations of the full-length chimeric or humanized immunoglobulin heavy and light chains.

TABLE 13

| Antibody | | |
|---|---|---|
| Name | Heavy Chain | Light Chain |
| Hu4F11-1 | Ch4F11 Chimeric | Ch4F11 Chimeric Kappa |
| | Heavy Variable + | Variable + Human |
| | Human IgG1 constant | Kappa constant |
| | (SEQ ID NO: 74) | (SEQ ID NO: 86) |
| Hu4F11-10 | Humanized Sh4F11 Hv3-23 | Ch4F11 Chimeric Kappa |
| | Heavy Human Variable + | Variable + Human |
| | Human IgG1 constant | Kappa constant |
| | (SEQ ID NO: 76) | (SEQ ID NO: 86) |
| Hu4F11-18 | Ch4F11 Chimeric | Humanized Hu4F11 Kv2D-29 |
| | Heavy Variable + | Human Variable + Human |
| | Human IgG1 constant | Kappa constant |
| | (SEQ ID NO: 74) | (SEQ ID NO: 88) |
| Hu4F11-32 | Humanized Sh4F11 Hv3-23 | Humanized Hu4F11 Kv2D-29 |
| | Heavy Human Variable + | Human Kappa Variable + |
| | Human IgG1 constant | Human Kappa constant |
| | (SEQ ID NO: 76) | (SEQ ID NO: 88) |
| Hu4F11-69 | Humanized Sh4F11 Hv3-23 | Humanized Hu4F11 Kv2D-29 |
| | A28T S31N T62S | N28H Human Kappa |
| | Heavy Human Variable + | Variable + Human |
| | Human IgG1 constant | Kappa constant |
| | (SEQ ID NO: 82) | (SEQ ID NO: 90) |
| Hu4F11-70 | Humanized Sh4F11 Hv3-23 | Humanized Hu4F11 Kv2D-29 |
| | A28T S31H T62S | N28H Human Kappa |
| | Heavy Human Variable + | Variable + Human |
| | Human IgG1 constant | Kappa constant |
| | (SEQ ID NO: 78) | (SEQ ID NO: 90) |
| Hu4F11-71 | Humanized Sh4F11 Hv3-23 | Humanized Hu4F11 Kv2D-29 |
| | A28T T62S | N28H Human Kappa |
| | Heavy Human Variable + | Variable + Human |
| | Human IgG1 constant | Kappa constant |
| | (SEQ ID NO: 84) | (SEQ ID NO: 90) |
| Hu4F11-72 | Humanized Sh4F11 Hv3-23 | Humanized Hu4F11 Kv2D-29 |
| | S31H T62S | N28H Human Kappa |
| | Heavy Human Variable + | Variable + Human |
| | Human IgG1 constant | Kappa constant |
| | (SEQ ID NO: 80) | (SEQ ID NO: 90) |
| Hu4F11-73 | Humanized Sh4F11 Hv3-23 | Humanized Hu4F11 Kv2D-29 |
| | A28T S31N T62S | N28Y Human Kappa |
| | Heavy Human Variable + | Variable + Human |
| | Human IgG1 constant | Kappa constant |
| | (SEQ ID NO: 82) | (SEQ ID NO: 94) |
| Hu4F11-74 | Humanized Sh4F11 Hv3-23 | Humanized Hu4F11 Kv2D-29 |
| | A28T S31H T62S | N28Y Human Kappa |
| | Heavy Human Variable + | Variable + Human |
| | Human IgG1 constant | Kappa constant |
| | (SEQ ID NO: 78) | (SEQ ID NO: 94) |
| Hu4F11-75 | Humanized Sh4F11 Hv3-23 | Humanized Hu4F11 Kv2D-29 |
| | A28T T62S | N28Y Human Kappa |
| | Heavy Human Variable + | Variable + Human |
| | Human IgG1 constant | Kappa constant |
| | (SEQ ID NO: 84) | (SEQ ID NO: 94) |
| Hu4F11-76 | Humanized Sh4F11 Hv3-23 | Humanized Hu4F11 Kv2D-29 |
| | S31H T62S | N28Y Human Kappa |
| | Heavy Human Variable + | Variable + Human |
| | Human IgG1 constant | Kappa constant |
| | (SEQ ID NO: 80) | (SEQ ID NO: 94) |
| Hu4F11-77 | Humanized Sh4F11 Hv3-23 | Humanized Hu4F11 Kv2D-29 |
| | A28T S31N T62S | N28Q Human Kappa |
| | Heavy Human Variable + | Variable + Human |
| | Human IgG1 constant | Kappa constant |
| | (SEQ ID NO: 82) | (SEQ ID NO: 92) |
| Hu4F11-78 | Humanized Sh4F11 Hv3-23 | Humanized Hu4F11 Kv2D-29 |
| | A28T S31H T62S | N28Q Human Kappa |
| | Heavy Human Variable + | Variable + Human |
| | Human IgG1 constant | Kappa constant |
| | (SEQ ID NO: 78) | (SEQ ID NO: 92) |
| Hu4F11-79 | Humanized Sh4F11 Hv3-23 | Humanized Hu4F11 Kv2D-29 |
| | A28T T62S | N28Q Human Kappa |
| | Heavy Human Variable + | Variable + Human |
| | Human IgG1 constant | Kappa constant |
| | (SEQ ID NO: 84) | (SEQ ID NO: 92) |
| Hu4F11-80 | Humanized Sh4F11 Hv3-23 | Humanized Hu4F11 Kv2D-29 |
| | S31H T62S | N28Q Human Kappa |
| | Heavy Human Variable + | Variable + Human |
| | Human IgG1 constant | Kappa constant |
| | (SEQ ID NO: 80) | (SEQ ID NO: 92) |

The antibody construct containing the full length chimeric heavy and light chains is designated below:
Chimeric 4F11 (Hu4F11-1)=Full Length Ch4F11 Chimeric Heavy Chain (Mouse Heavy Chain Variable Region and Human IgG1 Constant Region) (SEQ ID NO:74) plus Full Length Ch4F11 Chimeric Light Chain (Mouse Kappa Chain Variable Region and Human Kappa Constant Region) (SEQ ID NO:86)

Thirteen of the possible antibody constructs containing the full length immunoglobulin heavy and light chains containing humanized variable regions are designated below:
Hu4F11-32=Full Length Sh4F11 Hv3-23 Heavy Chain (Humanized Heavy Chain Variable Region and Human IgG1 Constant Region) (SEQ ID NO:76) plus Full Length Hu4F11 Kv2D-29 Light Chain (Humanized Kappa Chain Variable Region and Human Kappa Constant Region) (SEQ ID NO:88)
Hu4F11-69=Full Length Sh4F11 Hv3-23 A28T S31N T62S_Heavy Chain (Humanized Heavy Chain Variable Region and Human IgG1 Constant Region) (SEQ ID NO:82) plus Full Length Hu4F11 Kv2D-29 N28H Light Chain (Humanized Kappa Chain Variable Region and Human Kappa Constant Region) (SEQ ID NO:90)
Hu4F11-70=Full Length Sh4F11 Hv3-23 A28T S31H T62S Heavy Chain (Humanized Heavy Chain Variable Region and Human IgG1 Constant Region) (SEQ ID NO:78) plus Full Length Hu4F11 Kv2D-29 N28H Light Chain (Humanized Kappa Chain Variable Region and Human Kappa Constant Region) (SEQ ID NO:90)
Hu4F11-71=Full Length Sh4F11 Hv3-23 A28T T62S Heavy Chain (Humanized Heavy Chain Variable Region and Human IgG1 Constant Region) (SEQ ID NO:84) plus Full Length Hu4F11 Kv2D-29 N28H Light Chain (Humanized Kappa Chain Variable Region and Human Kappa Constant Region) (SEQ ID NO:90)
Hu4F11-72=Full Length Sh4F11 Hv3-23 S31H T62S Heavy Chain (Humanized Heavy Chain Variable Region and Human IgG1 Constant Region) (SEQ ID NO:80) plus Full Length Hu4F11 Kv2D-29 N28H Light Chain (Humanized Kappa Chain Variable Region and Human Kappa Constant Region) (SEQ ID NO:90)
Hu4F11-73=Full Length Sh4F11 Hv3-23 A28T S31N T62S_Heavy Chain (Humanized Heavy Chain Variable Region and Human IgG1 Constant Region) (SEQ ID NO:82) plus Full Length Hu4F11 Kv2D-29 N28Y Light Chain (Humanized Kappa Chain Variable Region and Human Kappa Constant Region) (SEQ ID NO:94)
Hu4F11-74=Full Length Sh4F11 Hv3-23 A28T S31H T62S Heavy Chain (Humanized Heavy Chain Variable Region and Human IgG1 Constant Region) (SEQ ID NO:78) plus Full Length Hu4F11 Kv2D-29 N28Y Light Chain (Humanized Kappa Chain Variable Region and Human Kappa Constant Region) (SEQ ID NO:94)
Hu4F11-75=Full Length Sh4F11 Hv3-23 A28T T62S Heavy Chain (Humanized Heavy Chain Variable Region and Human IgG1 Constant Region) (SEQ ID NO:84) plus Full Length Hu4F11 Kv2D-29 N28Y Light Chain (Humanized Kappa Chain Variable Region and Human Kappa Constant Region) (SEQ ID NO:94)
Hu4F11-76=Full Length Sh4F11 Hv3-23 S31H T62S Heavy Chain (Humanized Heavy Chain Variable Region and Human IgG1 Constant Region) (SEQ ID NO:80) plus Full Length Hu4F11 Kv2D-29 N28Y Light Chain (Humanized Kappa Chain Variable Region and Human Kappa Constant Region) (SEQ ID NO:94)
Hu4F11-77=Full Length Sh4F11 Hv3-23 A28T S31N T62S_Heavy Chain (Humanized Heavy Chain Variable Region and Human IgG1 Constant Region) (SEQ ID NO:82) plus Full Length Hu4F11 Kv2D-29 N28Q Light Chain (Humanized Kappa Chain Variable Region and Human Kappa Constant Region) (SEQ ID NO:92)
Hu4F11-78=Full Length Sh4F11 Hv3-23 A28T S31H T62S Heavy Chain (Humanized Heavy Chain Variable Region and Human IgG1 Constant Region) (SEQ ID NO:78) plus Full Length Hu4F11 Kv2D-29 N28Q Light Chain (Humanized Kappa Chain Variable Region and Human Kappa Constant Region) (SEQ ID NO:92)
Hu4F11-79=Full Length Sh4F11 Hv3-23 A28T T62S Heavy Chain (Humanized Heavy Chain Variable Region and Human IgG1 Constant Region) (SEQ ID NO:84) plus Full Length Hu4F11 Kv2D-29 N28Q Light Chain (Humanized Kappa Chain Variable Region and Human Kappa Constant Region) (SEQ ID NO:92)
Hu4F11-80=Full Length Sh4F11 Hv3-23 S31H T62S Heavy Chain (Humanized Heavy Chain Variable Region and Human IgG1 Constant Region) (SEQ ID NO:80) plus Full Length Hu4F11 Kv2D-29 N28Q Light Chain (Humanized Kappa Chain Variable Region and Human Kappa Constant Region) (SEQ ID NO:92)

### Example 9: Binding Affinities of Humanized and Chimeric Anti-Notch3 Monoclonal Antibodies

The binding affinities and kinetics of binding of chimeric and humanized antibodies to monomeric recombinant human Notch3 extracellular domain (containing EGF like domains 1-11) fusion protein (monomeric rhNotch3) and monomeric recombinant cynomolgous monkey Notch3 extracellular domain (containing EGF like domains 1-12) fusion protein (monomeric rcNotch3) were measured by surface plasmon resonance, using a BIAcore® T100 instrument (GE Healthcare, Piscataway, NJ).

Goat anti-human IgGs (Fc fragment specific, Jackson ImmunoResearch, West Grove, PA) were immobilized on carboxymethylated dextran CM4 sensor chips by amine coupling, according to a standard protocol. Analyses were performed at 25 and 37° C. using PBS containing 0.05% surfactant P20 as running buffer. The antibodies were captured in individual flow cells at a flow rate of 10 μL/minute. Injection time was varied for each antibody to yield an Rmax between 30 and 60 RU. Buffer or Notch3 protein diluted in running buffer was injected sequentially over a reference surface (no antibody captured) and the active surface (antibody to be tested) for 240 seconds at 60 μL/minute. The dissociation phase was monitored for up to 900 seconds. The surface was then regenerated with two 60-second injections of 10 mM Glycine-HCl, pH 2.25, at a flow rate of 30 μL/minute. The Notch3 concentration range tested was 50 nM to 6.25 nM.

Kinetic parameters were determined using the kinetic function of the BIAevaluation software (GE Healthcare) with double reference subtraction. Kinetic parameters for each antibody, kₐ (association rate constant), k_{d} (dissociation rate constant), and K_{D} (equilibrium dissociation constant) were determined. Kinetic values of purified monoclonal antibodies on monomeric rcNotch3 at 25° C. are summarized in Table 14.

TABLE 14

| Antibody | kₐ (1/Ms) | k_{d} (1/s) | K_{D} (M) | n |
|---|---|---|---|---|
| Hu4F11-1 | 4.8E+05 | 1.0E−02 | 2.4E−08 | 5 |
| Hu4F11-32 | 7.9E+05 | 7.5E−03 | 1.8E−08 | 8 |
| Hu4F11-69 | 1.2E+05 | 9.4E−04 | 7.7E−09 | 1 |
| Hu4F11-70 | 2.0E+05 | 1.0E−03 | 5.2E−09 | 1 |
| Hu4F11-71 | 1.5E+06 | 4.0E−03 | 2.7E−09 | 1 |
| Hu4F11-72 | 2.8E+06 | 4.5E−03 | 1.6E−09 | 1 |
| Hu4F11-73 | 7.6E+05 | 1.9E−03 | 2.6E−09 | 1 |
| Hu4F11-74 | 9.0E+05 | 2.5E−03 | 2.8E−09 | 1 |
| Hu4F11-75 | 7.2E+05 | 2.6E−03 | 3.7E−09 | 1 |
| Hu4F11-76 | 7.8E+05 | 1.9E−03 | 2.5E−09 | 1 |
| Hu4F11-77 | 9.6E+05 | 2.5E−03 | 2.6E−09 | 1 |
| Hu4F11-78 | 1.8E+06 | 3.8E−03 | 2.2E−09 | 1 |
| Hu4F11-79 | 1.3E+06 | 4.6E−03 | 3.4E−09 | 1 |
| Hu4F11-80 | 8.2E+05 | 3.2E−03 | 4.0E−09 | 1 |

Additional kinetic measurements were conducted for each antibody shown in Table 14. These measurements confirmed that the antibodies have affinities ranging from about lnM to about 25 nM for monomeric rcNotch3 at 25° C.

Kinetic values of purified monoclonal antibodies on monomeric rcNotch3 at 37° C. are summarized in Table 15.

TABLE 15

| Antibody | kₐ (1/Ms) | k_{d} (1/s) | K_{D} (M) | n |
|---|---|---|---|---|
| Hu4F11-32 | 5.0E+05 | 1.6E−02 | 3.2E−08 | 5 |
| Hu4F11-69 | 4.0E+05 | 3.8E−03 | 1.1E−08 | 3 |
| Hu4F11-70 | 3.7E+05 | 2.9E−03 | 9.1E−09 | 3 |
| Hu4F11-71 | 4.1E+05 | 5.6E−03 | 1.4E−08 | 3 |
| Hu4F11-72 | 4.4E+05 | 3.2E−03 | 9.8E−09 | 3 |
| Hu4F11-73 | 5.0E+05 | 6.3E−03 | 1.5E−08 | 3 |
| Hu4F11-74 | 4.4E+05 | 4.5E−03 | 1.2E−08 | 3 |
| Hu4F11-75 | 3.8E+05 | 8.9E−03 | 2.4E−08 | 3 |
| Hu4F11-76 | 5.4E+05 | 6.2E−03 | 1.2E−08 | 3 |
| Hu4F11-77 | 9.1E+05 | 1.0E−02 | 1.2E−08 | 3 |
| Hu4F11-78 | 6.6E+05 | 7.0E−03 | 1.1E−08 | 3 |
| Hu4F11-79 | 9.8E+05 | 2.1E−02 | 2.3E−08 | 3 |
| Hu4F11-80 | 9.1E+05 | 1.0E−02 | 1.2E−08 | 3 |

Additional kinetic measurements were conducted for each antibody shown in Table 15. These measurements confirmed that the antibodies have affinities ranging from about 9 nM to about 35 nM for monomeric rcNotch3 at 37° C.

Kinetic values of purified monoclonal antibodies on monomeric rhNotch3 at 37° C. are summarized in Table 16.

TABLE 16

| Antibody | kₐ (1/Ms) | k_{d} (1/s) | K_{D} (M) | n |
|---|---|---|---|---|
| Hu4F11-32 | 1.7E+06 | 4.6E−03 | 2.7E−09 | 1 |
| Hu4F11-69 | 1.3E+06 | 3.8E−03 | 2.9E−09 | 1 |
| Hu4F11-70 | 9.4E+05 | 2.8E−03 | 3.0E−09 | 1 |
| Hu4F11-71 | 1.2E+06 | 3.7E−03 | 3.0E−09 | 1 |
| Hu4F11-72 | 1.8E+05 | 9.4E−04 | 5.2E−09 | 1 |
| Hu4F11-73 | 2.1E+05 | 1.3E−03 | 5.9E−09 | 1 |
| Hu4F11-74 | 5.6E+05 | 2.2E−03 | 3.9E−09 | 1 |
| Hu4F11-75 | 2.7E+06 | 9.0E−03 | 3.4E−09 | 1 |
| Hu4F11-76 | 1.7E+06 | 5.5E−03 | 3.3E−09 | 1 |
| Hu4F11-77 | 2.2E+06 | 7.3E−03 | 3.3E−09 | 1 |
| Hu4F11-78 | 1.5E+06 | 5.5E−03 | 3.8E−09 | 1 |
| Hu4F11-79 | 1.8E+06 | 6.0E−03 | 3.2E−09 | 1 |
| Hu4F11-80 | 1.1E+06 | 4.1E−03 | 3.8E−09 | 1 |

Additional kinetic measurements were conducted for each antibody shown in Table 16. The additional measurements were averaged together with those presented in Table 16. These combined measurements indicated that the antibodies have affinities ranging from about 7 nM to about 16 nM for monomeric rhNotch3 at 37° C. (Table 17).

TABLE 17

| Antibody | kₐ (1/Ms) | k_{d} (1/s) | K_{D} (M) | n |
|---|---|---|---|---|
| Hu4F11-32 | 7.4E+06 | 8.4E−02 | 7.6E−09 | 2 |
| Hu4F11-69 | 7.1E+05 | 3.7E−03 | 8.5E−09 | 3 |
| Hu4F11-70 | 6.9E+05 | 3.9E−03 | 7.5E−09 | 4 |
| Hu4F11-71 | 1.1E+06 | 9.5E−03 | 9.2E−09 | 3 |
| Hu4F11-72 | 7.1E+05 | 6.9E−03 | 7.0E−09 | 4 |
| Hu4F11-73 | 4.9E+05 | 4.6E−03 | 1.1E−08 | 3 |
| Hu4F11-74 | 4.9E+05 | 5.1E−03 | 1.0E−08 | 4 |
| Hu4F11-75 | 1.7E+06 | 1.3E−02 | 1.0E−08 | 3 |
| Hu4F11-76 | 9.8E+05 | 6.4E−03 | 8.2E−09 | 3 |
| Hu4F11-77 | 1.2E+06 | 7.4E−03 | 8.6E−09 | 3 |
| Hu4F11-78 | 8.2E+05 | 6.0E−03 | 9.0E−09 | 4 |
| Hu4F11-79 | 9.0E+05 | 9.1E−03 | 1.6E−08 | 3 |
| Hu4F11-80 | 5.9E+05 | 5.6E−03 | 1.4E−08 | 3 |

The results in Tables 14-17 demonstrate that the chimeric and each of the humanized antibodies, have fast association rates (kₐ), very slow disassociation rates (k_{d}) and very high affinities (K_{D}). In particular, the antibodies have affinities ranging from about 1 nM to about 35 nM.

### Example 10: Binding of Humanized 4F11 Antibodies to Cell Surface-Expressed Notch3

Binding of humanized 4F11 variants to cell surface Notch3 receptor was confirmed by FACS. FlpIn™ CHO lines stably expressing human Notch3, or FlpIn™ CHO lines expressing no human Notch receptors, were used for binding experiments. CHO cells were harvested using Cell Dissociation Buffer (Life Technologies, Grand Island, NY) and resuspended at 2.5×10⁶ cells/ml in PBS/0.5% BSA. 100 μl of cell suspension per sample was added to a 96 well v-bottom plate. 4F11 humanized antibodies or human IgG were added at 5 μg/ml to the wells, mixed, and incubated on ice for one hour. After washing with PBS/0.5% BSA, anti-human PE conjugated secondary antibody was added at a 1:100 dilution in PBS/0.5% BSA and allowed to incubate on ice in the dark for 30 minutes. Cells were washed with PBS/0.5% BSA, then resuspended in 300 ul PBS/0.5% BSA and FACS analysis was performed. Expression of human Notch3 receptor was confirmed using anti-human Notch3 PE (BioLegend, San Diego, CA) as a positive control. FACS analysis confirmed that the humanized 4F11 antibody variants bind to human Notch3 expressed on the cell surface.

### Example 11: Inhibition of Ligand-Induced Notch3 ICD Cleavage by Humanized 4F11 Antibodies

Selected humanized 4F11 variants were tested for their ability to inhibit ligand-induced activation of Notch3, as measured by the presence of cleaved ICD. The MDA-MB-468 breast cancer cell line, which expresses endogenous human Notch3, was plated in 96-well plates previously coated with hJag2-mFc fusion protein. The wells were prepared by diluting α-mouse Fc (Jackson ImmunoResearch, West Grove, PA) to 5 μg/ml in sterile-filtered carbonate-bicarbonate coating buffer, pH 9.4 (Thermo Fisher Scientific, Rockland, MD). Then 100 μl of the diluted antibody was added to each well of a 96-well Maxisorp™ plate and incubated overnight at 4° C. The next day, wells were washed three times with PBS/0.5% BSA before adding 100 μl of soluble Jag2-mFc fusion protein or mouse IgG Fc (Jackson Immunolabs) diluted to 5 μg/ml in PBS/0.5% BSA. After incubating for two hours at room temperature on an orbital shaker, the wells were washed three times with PBS/05% BSA to remove unbound ligand. MDA-MB-468 cells were counted and resuspended in fresh growth media at 0.4×10⁶ cells/ml. Cells were pre-incubated with 10 μg/ml of the 4F11 humanized antibodies for 30 minutes at 37° C., before seeding 100 μl of the suspension into 96-well plates coated with hJag2-mFc ligand or mFc. Cells were incubated overnight at 37° C. The next day wells were gently washed with ice cold PBS, then cells harvested by adding RIPA buffer containing protease inhibitors directly to the well. Lysates were clarified by centrifugation in a refrigerated microcentrifuge. Supernatants were boiled with 5×SDS sample buffer before loading onto an SDS PAGE gel and Western blotting. Blots were probed with a α-Notch3 antibody to detect the cleaved intracellular domain (Cell Signaling, Danvers, MA). The same blots were also probed with anti-β tubulin (Cell Signaling) for use as a loading control. Bands were quantitated using ImageLab software (BioRad, Hercules, CA) and values adjusted relative to their respective loading control. Each sample was then normalized relative to the value of the Notch3 ICD band from cells plated on wells without ligand in the presence of hIgG control antibody.

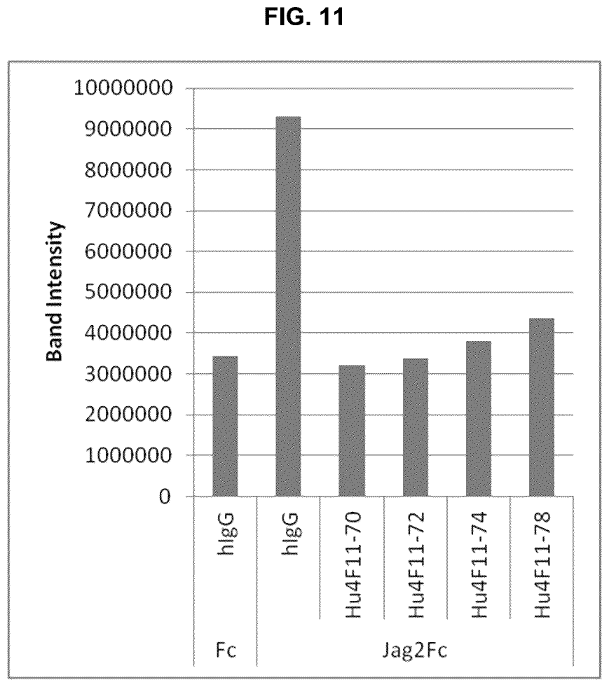

The humanized 4F11 antibody variants significantly inhibit ligand-induced cleavage of the Notch3 ICD as summarized in FIG. 11.

### Example 12: Inhibition of Notch3-Dependent Transcription by Humanized 4F11 Antibodies

Luciferase reporter assays were used to assess the ability of certain humanized 4F11 antibodies to inhibit ligand-dependent Notch3 receptor signaling and transcriptional activity. HCC1143 reporter lines co-cultured with CHO cells expressing Notch ligands were utilized for these assays.

Stable lines expressing full length Notch ligands at the cell surface were produced by transfecting FlpIn™ CHO cells with Jag1, Jag2, DLL1, or DLL4 cDNAs cloned into the pcDNA5FRT vector using Lipofectamine 2000 (Life Technologies) according to the manufacturer's protocol. Twenty-four hours after transfection, CHO cells were split into F12 media containing 10% FBS, 2 mM L-Glutamine and 700 μg/ml hygromycin B (Sigma-Aldrich, St. Louis, MO) to select for transfected cells. Expression of Notch ligands was confirmed by FACS analysis using antibodies against Jag1 (R&D Systems, Inc., Minneapolis, MN), Jag2 (R&D Systems), DLL1 (R&D Systems), and DLL4 (BioLegend, San Diego, CA).

FlpIn™ CHO lines stably expressing Notch ligands were trypsinized, counted and seeded in 96-well plates at 60K cells/well in 100 μl of Ham's F12 K media without hygromycin B. The next day, HCC1143 reporter cells were spun down and resuspended in RPMI media containing 2% FBS at 0.2×10⁶ cells/ml. Antibodies were serially diluted into RPMI without FBS, and 100 μl of antibody solution was added to an equal volume of reporter cells for 30 minutes at 37° C. in a 5% CO2 incubator. After removing media from the hJag2 CHO cells, 100 μl of the HCC1143 reporter/antibody mix was added to the ligand-expressing cells and allowed to incubate at 37° C. overnight. Twenty-four hours later, 96-well plates placed at room temperature for 20 minutes, then processed with the Bright Glo (Promega, Madison, WI) reporter assay protocol per the manufacturer's instructions. Lysates were transferred to white-walled 96 well plates (Greiner Bio-One, Frickenhausen, Germany) and read on a GloMax Luminometer (Promega) using the Bright Glo program.

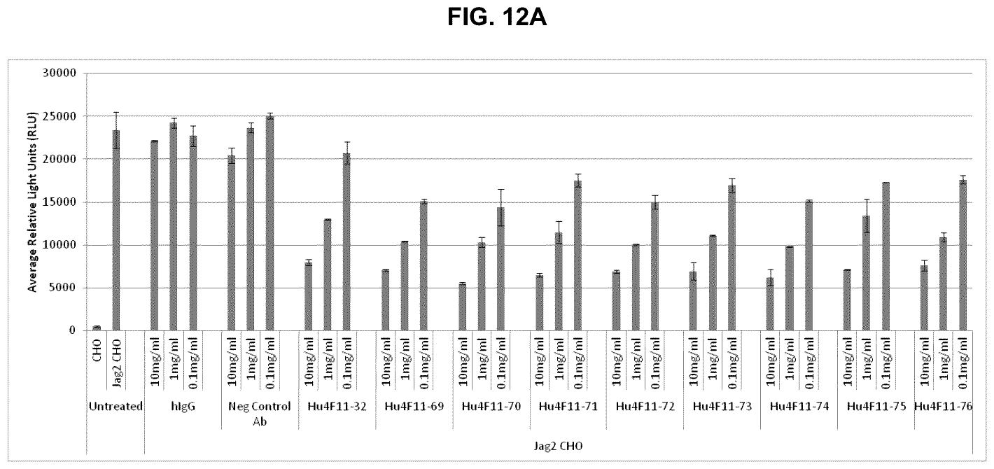

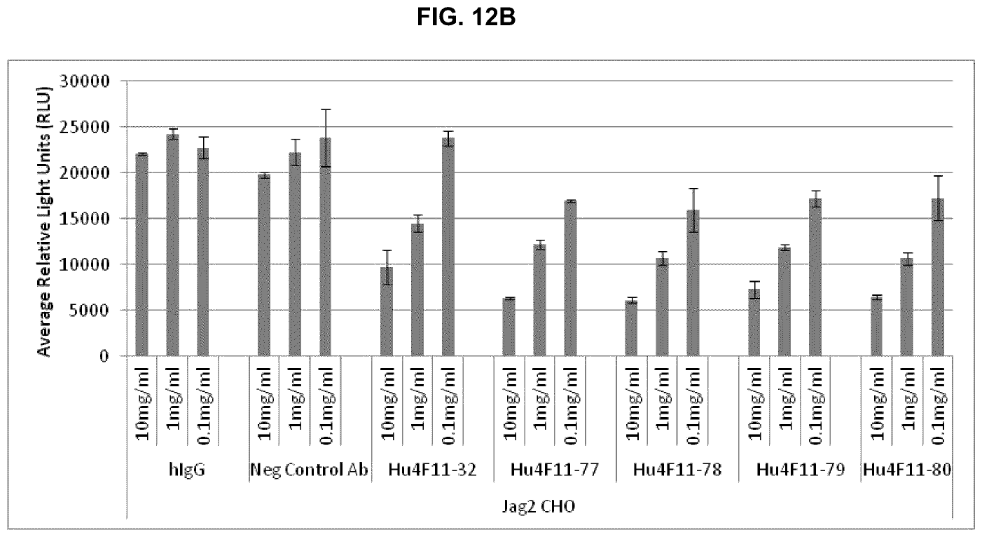

As shown in FIG. 12A-B, the humanized 4F11 antibody variants inhibit activation of ligand-induced transcription in the HCC1143 reporter cell line.

### Example 13: Inhibition of Notch3 ICD Cleavage In Vivo

Selected humanized 4F11 antibodies (i.e., Hu4F11-70, Hu4F11-72, Hu4F11-78) were tested for their ability to inhibit cleavage of Notch3 ICD expressed in HCC2429 tumors in vivo.

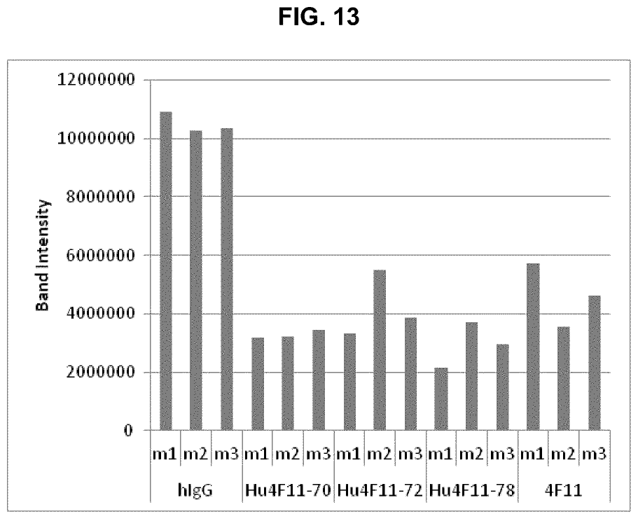

All mice were treated in accordance with the OLAW Public Health Service Policy on Human Care and Use of Laboratory Animals and the ILAR Guide for the Care and Use of Laboratory Animals. All in vivo studies were conducted following the protocols approved by the AVEO Institutional Animal Care and Use Committee. HCC2429 is a lung cancer cell line harboring a translocation of chromosome 19 that results in overexpression of the Notch3 receptor. For the Notch3 ICD cleavage experiments, approximately thirteen week old NCR nude mice (Taconic, Germantown, NY) were inoculated subcutaneously into the right flank with 5×10⁶ cells in 1:1 DMEM+Matrigel (Invitrogen, Carlsbad, CA)/Matrigel (BD Biosciences, San Jose, CA). Tumor measurements were taken twice weekly, using vernier calipers. Tumor volume was calculated using the formula: V=0.5×width×width×length. When tumors approached a volume of 300-400 mm³, mice were randomized into groups of three animals each (corresponds to mouse 1 (m1) through mouse 3 (m3), as designated in FIG. 13). The next day, mice were treated with 20 mg/kg hIgG (control), or 20 mg/kg of antibodies Hu4F11-70, Hu4F11-72, Hu4F11-78 or murine 4F11 (mu4F11) by intraperitoneal injection. Mice were dosed once, and tumors were collected 24 hours later and snap frozen.

To assess levels of cleaved Notch3 ICD, tumors were pulverized using a Covaris cryoPREP™ impactor (Covaris, Woburn, MA), resuspended in RIPA buffer (Boston BioProducts) containing protease inhibitors, and rotated at 4° C. for 1 hour. Lysates were clarified by spinning at 14 k rpm for 15 minutes in a refrigerated microcentrifuge. Protein concentration for each sample was measured using the BioRad DC protein assay (BioRad). Equal concentrations of protein from each sample were loaded onto an SDS PAGE gel and transferred onto nitrocellulose by Western blotting. Blots were probed with antibody against the C-terminus of Notch3 (Cell Signaling) to detect levels of the cleaved ICD. Blots were also probed with antibody against β-tubulin (Cell Signaling) for use as a loading control. Bands were quantified using Image Lab 3.0 software (BioRad), and the intensity of Notch3 ICD bands were normalized to their respective β-tubulin loading control. All humanized 4F11 antibodies tested (i.e., Hu4F11-70, Hu4F11-72, Hu4F11-78) significantly inhibit Notch3 activation in vivo, as measured by Notch3 ICD levels present in tumors 24 hours after single dose antibody treatment (FIG. 13).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

1-50. (canceled)

51. A method of inhibiting or reducing ligand-induced Notch3 activity, comprising administering 0.5-20 mg/kg of an anti-Notch3 antibody, or antigen-binding fragment thereof, to a mammal, wherein the anti-Notch3 antibody comprises an immunoglobulin heavy chain variable region (VH) and an immunoglobulin light chain variable region (VL) selected from the group consisting of:
(a) (i) a VH comprising a CDR_{H1} comprising the amino acid sequence selected from the group consisting of SEQ ID NO: 5 and SEQ ID NO:11, a CDR_{H2} comprising the amino acid sequence of SEQ ID NO:6, and a CDR_{H3} comprising the amino acid sequence of SEQ ID NO:7; and
(ii) a VL comprising a CDR_{L1} comprising the amino acid sequence of SEQ ID NO:8, a CDR_{L2} comprising the amino acid sequence of SEQ ID NO:9, and a CDR_{L3} comprising the amino acid sequence of SEQ ID NO:10;
(b) (i) a VH comprising a CDR_{H1} comprising the amino acid sequence selected from the group consisting of SEQ ID NO: 5 and SEQ ID NO:11, a CDR_{H2} comprising the amino acid sequence of SEQ ID NO:6, and a CDR_{H3} comprising the amino acid sequence of SEQ ID NO:7; and
(ii) a VL comprising a CDR_{L1} comprising the amino acid sequence of SEQ ID NO:62, a CDR_{L2} comprising the amino acid sequence of SEQ ID NO:9, and a CDR_{L3} comprising the amino acid sequence of SEQ ID NO:10;
(c) (i) a VH comprising a CDR_{H1} comprising the amino acid sequence selected from the group consisting of SEQ ID NO: 5 and SEQ ID NO:11, a CDR_{H2} comprising the amino acid sequence of SEQ ID NO:6, and a CDR_{H3} comprising the amino acid sequence of SEQ ID NO:7; and
(ii) a VL comprising a CDR_{L1} comprising the amino acid sequence of SEQ ID NO:63, a CDR_{L2} comprising the amino acid sequence of SEQ ID NO:9, and a CDR_{L3} comprising the amino acid sequence of SEQ ID NO:10;
(d) (i) a VH comprising a CDR_{H1} comprising the amino acid sequence selected from the group consisting of SEQ ID NO: 5 and SEQ ID NO:11, a CDR_{H2} comprising the amino acid sequence of SEQ ID NO:6, and a CDR_{H3} comprising the amino acid sequence of SEQ ID NO:7; and
(ii) a VL comprising a CDR_{L1} comprising the amino acid sequence of SEQ ID NO:64, a CDR_{L2} comprising the amino acid sequence of SEQ ID NO:9, and a CDR_{L3} comprising the amino acid sequence of SEQ ID NO:10;
(e) (i) a VH comprising a CDR_{H1} comprising the amino acid sequence of SEQ ID NO:51, a CDR_{H2} comprising the amino acid sequence of SEQ ID NO:53, and a CDR_{H3} comprising the amino acid sequence of SEQ ID NO:7;
(ii) a VL comprising a CDR_{L1} comprising the amino acid sequence of SEQ ID NO:8, a CDR_{L2} comprising the amino acid sequence of SEQ ID NO:9, and a CDR_{L3} comprising the amino acid sequence of SEQ ID NO:10;
(f) (i) a VH comprising a CDR_{H1} comprising the amino acid sequence of SEQ ID NO:51, a CDR_{H2} comprising the amino acid sequence of SEQ ID NO:53, and a CDR_{H3} comprising the amino acid sequence of SEQ ID NO:7; and
(ii) a VL comprising a CDR_{L1} comprising the amino acid sequence of SEQ ID NO:63, a CDR_{L2} comprising the amino acid sequence of SEQ ID NO:9, and a CDR_{L3} comprising the amino acid sequence of SEQ ID NO:10;
(g) (i) a VH comprising a CDR_{H1} comprising the amino acid sequence selected from the group consisting of SEQ ID NO: 52 and SEQ ID NO:56, a CDR_{H2} comprising the amino acid sequence of SEQ ID NO:53, and a CDR_{H3} comprising the amino acid sequence of SEQ ID NO:7; and
(ii) a VL comprising a CDR_{L1} comprising the amino acid sequence of SEQ ID NO:8, a CDR_{L2} comprising the amino acid sequence of SEQ ID NO:9, and a CDR_{L3} comprising the amino acid sequence of SEQ ID NO:10;
(h) (i) a VH comprising a CDR_{H1} comprising the amino acid sequence selected from the group consisting of SEQ ID NO: 52 and SEQ ID NO:56, a CDR_{H2} comprising the amino acid sequence of SEQ ID NO:53, and a CDR_{H3} comprising the amino acid sequence of SEQ ID NO:7; and
(ii) a VL comprising a CDR_{L1} comprising the amino acid sequence of SEQ ID NO:62, a CDR_{L2} comprising the amino acid sequence of SEQ ID NO:9, and a CDR_{L3} comprising the amino acid sequence of SEQ ID NO:10;
(i) (i) a VH comprising a CDR_{H1} comprising the amino acid sequence selected from the group consisting of SEQ ID NO: 52 and SEQ ID NO:56, a CDR_{H2} comprising the amino acid sequence of SEQ ID NO:53, and a CDR_{H3} comprising the amino acid sequence of SEQ ID NO:7; and
(ii) a VL comprising a CDR_{L1} comprising the amino acid sequence of SEQ ID NO:63, a CDR_{L2} comprising the amino acid sequence of SEQ ID NO:9, and a CDR_{L3} comprising the amino acid sequence of SEQ ID NO:10;
(j) (i) a VH comprising a CDR_{H1} comprising the amino acid sequence selected from the group consisting of SEQ ID NO: 52 and SEQ ID NO:56, a CDR_{H2} comprising the amino acid sequence of SEQ ID NO:53, and a CDR_{H3} comprising the amino acid sequence of SEQ ID NO:7; and
(ii) a VL comprising a CDR_{L1} comprising the amino acid sequence of SEQ ID NO:64, a CDR_{L2} comprising the amino acid sequence of SEQ ID NO:9, and a CDR_{L3} comprising the amino acid sequence of SEQ ID NO:10;
(k) (i) a VH comprising a CDR_{H1} comprising the amino acid sequence selected from the group consisting of SEQ ID NO: 5 and SEQ ID NO:57, a CDR_{H2} comprising the amino acid sequence of SEQ ID NO:53, and a CDR_{H3} comprising the amino acid sequence of SEQ ID NO:7; and
(ii) a VL comprising a CDR_{L1} comprising the amino acid sequence of SEQ ID NO:8, a CDR_{L2} comprising the amino acid sequence of SEQ ID NO:9, and a CDR_{L3} comprising the amino acid sequence of SEQ ID NO:10;
(l) (i) a VH comprising a CDR_{H1} comprising the amino acid sequence selected from the group consisting of SEQ ID NO: 5 and SEQ ID NO:57, a CDR_{H2} comprising the amino acid sequence of SEQ ID NO:53, and a CDR_{H3} comprising the amino acid sequence of SEQ ID NO:7; and
(ii) a VL comprising a CDR_{L1} comprising the amino acid sequence of SEQ ID NO:62, a CDR_{L2} comprising the amino acid sequence of SEQ ID NO:9, and a CDR_{L3} comprising the amino acid sequence of SEQ ID NO:10;
(m) (i) a VH comprising a CDR_{H1} comprising the amino acid sequence selected from the group consisting of SEQ ID NO: 5 and SEQ ID NO:57, a CDR_{H2} comprising the amino acid sequence of SEQ ID NO:53, and a CDR_{H3} comprising the amino acid sequence of SEQ ID NO:7; and
(ii) a VL comprising a CDR_{L1} comprising the amino acid sequence of SEQ ID NO:63, a CDR_{L2} comprising the amino acid sequence of SEQ ID NO:9, and a CDR_{L3} comprising the amino acid sequence of SEQ ID NO:10; and
(n) (i) a VH comprising a CDR_{H1} comprising the amino acid sequence selected from the group consisting of SEQ ID NO: 5 and SEQ ID NO:57, a CDR_{H2} comprising the amino acid sequence of SEQ ID NO:53, and a CDR_{H3} comprising the amino acid sequence of SEQ ID NO:7; and
(ii) a VL comprising a CDR_{L1} comprising the amino acid sequence of SEQ ID NO:64, a CDR_{L2} comprising the amino acid sequence of SEQ ID NO:9, and a CDR_{L3} comprising the amino acid sequence of SEQ ID NO:10.

52. The method of claim 51, wherein the ligand-induced Notch3 activity is ligand-induced Notch3 intracellular domain (ICD) cleavage.

53. The method of claim 51, wherein the ligand-induced Notch3 activity is ligand-induced Notch3 receptor signaling and/or ligand-induced Notch3-mediated transcription.

54. The method of claim 51, wherein the ligand is selected from the group consisting of Jag1, Jag2, DLL1, and DLL2.

55. The method of claim 51, wherein the mammal is a human.

56. The method of claim 55, wherein the human is a human cancer patient.

57. The method of claim 51, wherein the VH and the VL are selected from the group consisting of:
(a) a VH comprising the amino acid sequence of SEQ ID NO:2, and a VL comprising the amino acid sequence of SEQ ID NO:4;
(b) a VH comprising the amino acid sequence of SEQ ID NO:34, and a VL comprising the amino acid sequence of SEQ ID NO:4;
(c) a VH comprising the amino acid sequence of SEQ ID NO:2, and a VL comprising the amino acid sequence of SEQ ID NO:44;
(d) a VH comprising the amino acid sequence of SEQ ID NO:34, and a VL comprising the amino acid sequence of SEQ ID NO:44;
(e) a VH comprising the amino acid sequence of SEQ ID NO:2, and a VL comprising the amino acid sequence of SEQ ID NO:46;
(f) a VH comprising the amino acid sequence of SEQ ID NO:34, and a VL comprising the amino acid sequence of SEQ ID NO:46;
(g) a VH comprising the amino acid sequence of SEQ ID NO:2, and a VL comprising the amino acid sequence of SEQ ID NO:48;
(h) a VH comprising the amino acid sequence of SEQ ID NO:34, and a VL comprising the amino acid sequence of SEQ ID NO:48;
(i) a VH comprising the amino acid sequence of SEQ ID NO:2, and a VL comprising the amino acid sequence of SEQ ID NO:50;
(j) a VH comprising the amino acid sequence of SEQ ID NO:34, and a VL comprising the amino acid sequence of SEQ ID NO:50;
(k) a VH comprising the amino acid sequence of SEQ ID NO:36, and a VL comprising the amino acid sequence of SEQ ID NO:4;
(l) a VH comprising the amino acid sequence of SEQ ID NO:38, and a VL comprising the amino acid sequence of SEQ ID NO:4;
(m) a VH comprising the amino acid sequence of SEQ ID NO:36, and a VL comprising the amino acid sequence of SEQ ID NO:44;
(n) a VH comprising the amino acid sequence of SEQ ID NO:38, and a VL comprising the amino acid sequence of SEQ ID NO:44;
(o) a VH comprising the amino acid sequence of SEQ ID NO:36, and a VL comprising the amino acid sequence of SEQ ID NO:48;
(p) a VH comprising the amino acid sequence of SEQ ID NO:38, and a VL comprising the amino acid sequence of SEQ ID NO:48;
(q) a VH comprising the amino acid sequence of SEQ ID NO:40, and a VL comprising the amino acid sequence of SEQ ID NO:4;
(r) a VH comprising the amino acid sequence of SEQ ID NO:40, and a VL comprising the amino acid sequence of SEQ ID NO:44;
(s) a VH comprising the amino acid sequence of SEQ ID NO:40, and a VL comprising the amino acid sequence of SEQ ID NO:46;
(t) a VH comprising the amino acid sequence of SEQ ID NO:40, and a VL comprising the amino acid sequence of SEQ ID NO:48;
(u) a VH comprising the amino acid sequence of SEQ ID NO:40, and a VL comprising the amino acid sequence of SEQ ID NO:50;
(v) a VH comprising the amino acid sequence of SEQ ID NO:42, and a VL comprising the amino acid sequence of SEQ ID NO:4;
(w) a VH comprising the amino acid sequence of SEQ ID NO:42, and a VL comprising the amino acid sequence of SEQ ID NO:44;
(x) a VH comprising the amino acid sequence of SEQ ID NO:42, and a VL comprising the amino acid sequence of SEQ ID NO:46;
(y) a VH comprising the amino acid sequence of SEQ ID NO:42, and a VL comprising the amino acid sequence of SEQ ID NO:48; and
(z) a VH comprising the amino acid sequence of SEQ ID NO:42, and a VL comprising the amino acid sequence of SEQ ID NO:50.

58. The method of claim 51, wherein the anti-Notch3 antibody comprises an immunoglobulin heavy chain (HC) and an immunoglobulin light chain (LC) selected from the group consisting of:
(a) an HC comprising the amino acid sequence of SEQ ID NO: 27, and an LC comprising the amino acid sequence of SEQ ID NO: 29;
(b) an HC comprising the amino acid sequence of SEQ ID NO: 74, and an LC comprising the amino acid sequence of SEQ ID NO: 86;
(c) an HC comprising the amino acid sequence of SEQ ID NO: 74, and an LC comprising the amino acid sequence of SEQ ID NO: 88;
(d) an HC comprising the amino acid sequence of SEQ ID NO: 76, and an LC comprising the amino acid sequence of SEQ ID NO: 86;
(e) an HC comprising the amino acid sequence of SEQ ID NO: 76, and an LC comprising the amino acid sequence of SEQ ID NO: 88;
(f) an HC comprising the amino acid sequence of SEQ ID NO: 82, and an LC comprising the amino acid sequence of SEQ ID NO: 90;
(g) an HC comprising the amino acid sequence of SEQ ID NO: 84, and an LC comprising the amino acid sequence of SEQ ID NO: 90;
(h) an HC comprising the amino acid sequence of SEQ ID NO: 78, and an LC comprising the amino acid sequence of SEQ ID NO: 92;
(i) an HC comprising the amino acid sequence of SEQ ID NO: 80, and an LC comprising the amino acid sequence of SEQ ID NO: 92;
(j) an HC comprising the amino acid sequence of SEQ ID NO: 82, and an LC comprising the amino acid sequence of SEQ ID NO: 92;
(k) an HC comprising the amino acid sequence of SEQ ID NO: 84, and an LC comprising the amino acid sequence of SEQ ID NO: 92;
(l) an HC comprising the amino acid sequence of SEQ ID NO: 80, and an LC comprising the amino acid sequence of SEQ ID NO: 94;
(m) an HC comprising the amino acid sequence of SEQ ID NO: 82, and an LC comprising the amino acid sequence of SEQ ID NO: 94; and
(n) an HC comprising the amino acid sequence of SEQ ID NO: 84, and an LC comprising the amino acid sequence of SEQ ID NO: 94.

59. A method of inhibiting or reducing ligand-induced Notch3 activity, comprising administering 0.5-20 mg/kg of an anti-Notch3 antibody, or antigen-binding fragment thereof, to a mammal, wherein the anti-Notch3 antibody comprises the HC-CDR1, HC-CDR2, and HC-CDR3 amino acid sequences of a VH and comprises the LC-CDR1, LC-CDR2 and LC-CDR3 amino acid sequences of a VL wherein:
(a) the VH comprises the amino acid sequence of SEQ ID NO:2, and the VL comprises the amino acid sequence of SEQ ID NO:4;
(b) the VH comprises the amino acid sequence of SEQ ID NO:34, and the VL comprises the amino acid sequence of SEQ ID NO:4;
(c) the VH comprises the amino acid sequence of SEQ ID NO:2, and the VL comprises the amino acid sequence of SEQ ID NO:44;
(d) the VH comprises the amino acid sequence of SEQ ID NO:34, and the VL comprises the amino acid sequence of SEQ ID NO:44;
(e) the VH comprises the amino acid sequence of SEQ ID NO:2, and the VL comprises the amino acid sequence of SEQ ID NO:46;
(f) the VH comprises the amino acid sequence of SEQ ID NO:34, and the VL comprises the amino acid sequence of SEQ ID NO:46;
(g) the VH comprises the amino acid sequence of SEQ ID NO:2, and the VL comprises the amino acid sequence of SEQ ID NO:48;
(h) the VH comprises the amino acid sequence of SEQ ID NO:34, and the VL comprises the amino acid sequence of SEQ ID NO:48;
(i) the VH comprises the amino acid sequence of SEQ ID NO:2, and the VL comprises the amino acid sequence of SEQ ID NO:50;
(j) the VH comprises the amino acid sequence of SEQ ID NO:34, and the VL comprises the amino acid sequence of SEQ ID NO:50;
(k) the VH comprises the amino acid sequence of SEQ ID NO:36, and the VL comprises the amino acid sequence of SEQ ID NO:4;
(l) the VH comprises the amino acid sequence of SEQ ID NO:38, and the VL comprises the amino acid sequence of SEQ ID NO:4;
(m) the VH comprises the amino acid sequence of SEQ ID NO:36, and the VL comprises the amino acid sequence of SEQ ID NO:44;
(n) the VH comprises the amino acid sequence of SEQ ID NO:38, and the VL comprises the amino acid sequence of SEQ ID NO:44;
(o) the VH comprises the amino acid sequence of SEQ ID NO:36, and the VL comprises the amino acid sequence of SEQ ID NO:48;
(p) the VH comprises the amino acid sequence of SEQ ID NO:38, and the VL comprises the amino acid sequence of SEQ ID NO:48;
(q) the VH comprises the amino acid sequence of SEQ ID NO:40, and the VL comprises the amino acid sequence of SEQ ID NO:4;
(r) the VH comprises the amino acid sequence of SEQ ID NO:40, and the VL comprises the amino acid sequence of SEQ ID NO:44;
(s) the VH comprises the amino acid sequence of SEQ ID NO:40, and the VL comprises the amino acid sequence of SEQ ID NO:46;
(t) the VH comprises the amino acid sequence of SEQ ID NO:40, and the VL comprises the amino acid sequence of SEQ ID NO:48;
(u) the VH comprises the amino acid sequence of SEQ ID NO:40, and the VL comprises the amino acid sequence of SEQ ID NO:50;
(v) the VH comprises the amino acid sequence of SEQ ID NO:42, and the VL comprises the amino acid sequence of SEQ ID NO:4;
(w) the VH comprises the amino acid sequence of SEQ ID NO:42, and the VL comprises the amino acid sequence of SEQ ID NO:44;
(x) the VH comprises the amino acid sequence of SEQ ID NO:42, and the VL comprises the amino acid sequence of SEQ ID NO:46;
(y) the VH comprises the amino acid sequence of SEQ ID NO:42, and the VL comprises the amino acid sequence of SEQ ID NO:48; and
(z) the VH comprises the amino acid sequence of SEQ ID NO:42, and the VL comprises the amino acid sequence of SEQ ID NO:50.

60. The method of claim 59, wherein the ligand-induced Notch3 activity is ligand-induced Notch3 intracellular domain (ICD) cleavage.

61. The method of claim 59, wherein the ligand-induced Notch3 activity is ligand-induced Notch3 receptor signaling and/or ligand-induced Notch3-mediated transcription.

62. The method of claim 59, wherein the ligand is selected from the group consisting of Jag1, Jag2, DLL1, and DLL2.

63. The method of claim 59, wherein the mammal is a human.

64. The method of claim 63, wherein the human is a human cancer patient.

65. The method of claim 51, wherein the method comprises administering 20 mg/kg of the anti-Notch3 antibody.

66. The method of claim 59, wherein the method comprises administering 20 mg/kg of the anti-Notch3 antibody.


SEQUENCE LISTING (96 sequences)

SEQ ID NO: 1 - DNA, 360 bp, synthetic construct
  misc_feature 1..360: note = source = /note="Description of Artificial Sequence: Synthetic polynucleotide"
  source 1..360: mol_type = other DNA
gaagtgcagctggtggagtctgggggaggcttagtgaagcctggagggtccctgaaactctcctgtgcagcctctggattcgctttcagtagctatgacatgtcttgggttcgccagactccggagaagaggctggagtgggtcgcatacattagtcgtggtggtggtagcacctactatccagacactgtgaagggccgattcaccatctccagagacaatgccaagaacaccctgtacctgcaaatgagcagtctgaagtctgaggacacagccatgtattactgtggaagacatgctactacggcctactggtacttcgatgtctggggcgcagggaccacggtcaccgtctcctca

SEQ ID NO: 2 - protein, 120 aa, synthetic construct
  REGION 1..120: note = source = /note="Description of Artificial Sequence: Synthetic polypeptide"
EVQLVESGGGLVKPGGSLKLSCAASGFAFSSYDMSWVRQTPEKRLEWVAYISRGGGSTYYPDTVKGRFTISRDNAKNTLYLQMSSLKSEDTAMYYCGRHATTAYWYFDVWGAGTTVTVSS

SEQ ID NO: 3 - DNA, 336 bp, synthetic construct
  misc_feature 1..336: note = source = /note="Description of Artificial Sequence: Synthetic polynucleotide"
  source 1..336: mol_type = other DNA
gatgttgtgatgacccaaactccactctccctgcctgtcagtcttggagatcaagcctccatctcttgcagatctagtcagagccttgtacacactaatggcaacacctatttacattggtacctgcagaagccaggccagtctccaaaactcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 4 - protein, 112 aa, synthetic construct
  REGION 1..112: note = source = /note="Description of Artificial Sequence: Synthetic polypeptide"
DVVMTQTPLSLPVSLGDQASISCRSSQSLVHTNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 5 - protein, 5 aa, synthetic construct
  REGION 1..5: note = source = /note="Description of Artificial Sequence: Synthetic peptide"
SYDMS

SEQ ID NO: 6 - protein, 17 aa, synthetic construct
  REGION 1..17: note = source = /note="Description of Artificial Sequence: Synthetic peptide"
YISRGGGSTYYPDTVKG

SEQ ID NO: 7 - protein, 11 aa, synthetic construct
  REGION 1..11: note = source = /note="Description of Artificial Sequence: Synthetic peptide"
HATTAYWYFDV

SEQ ID NO: 8 - protein, 16 aa, synthetic construct
  REGION 1..16: note = source = /note="Description of Artificial Sequence: Synthetic peptide"
RSSQSLVHTNGNTYLH

SEQ ID NO: 9 - protein, 7 aa, synthetic construct
  REGION 1..7: note = source = /note="Description of Artificial Sequence: Synthetic peptide"
KVSNRFS

SEQ ID NO: 10 - protein, 9 aa, synthetic construct
  REGION 1..9: note = source = /note="Description of Artificial Sequence: Synthetic peptide"
SQSTHVPWT

SEQ ID NO: 11 - protein, 7 aa, synthetic construct
  REGION 1..7: note = source = /note="Description of Artificial Sequence: Synthetic peptide"
GFAFSSY

SEQ ID NO: 12 - protein, 6 aa, synthetic construct
  REGION 1..6: note = source = /note="Description of Artificial Sequence: Synthetic peptide"
SRGGGS

SEQ ID NO: 13 - DNA, 45 bp, synthetic construct
  misc_feature 1..45: note = source = /note="Description of Artificial Sequence: Synthetic primer"
  source 1..45: mol_type = other DNA
ctaatacgactcactatagggcaagcagtggtatcaacgcagagt

SEQ ID NO: 14 - protein, 8 aa, synthetic construct
  REGION 1..8: note = source = /note="Description of Artificial Sequence: Synthetic peptide"
GFAFSSYD

SEQ ID NO: 15 - protein, 8 aa, synthetic construct
  REGION 1..8: note = source = /note="Description of Artificial Sequence: Synthetic peptide"
ISRGGGST

SEQ ID NO: 16 - protein, 13 aa, synthetic construct
  REGION 1..13: note = source = /note="Description of Artificial Sequence: Synthetic peptide"
GRHATTAYWYFDV

SEQ ID NO: 17 - DNA, 22 bp, synthetic construct
  misc_feature 1..22: note = source = /note="Description of Artificial Sequence: Synthetic primer"
  source 1..22: mol_type = other DNA
ctaatacgactcactatagggc

SEQ ID NO: 18 - DNA, 21 bp, synthetic construct
  misc_feature 1..21: note = source = /note="Description of Artificial Sequence: Synthetic primer"
  source 1..21: mol_type = other DNA
tatgcaaggcttacaaccaca

SEQ ID NO: 19 - DNA, 23 bp, synthetic construct
  misc_feature 1..23: note = source = /note="Description of Artificial Sequence: Synthetic primer"
  source 1..23: mol_type = other DNA
cgactgaggcacctccagatgtt

SEQ ID NO: 20 - protein, 11 aa, synthetic construct
  REGION 1..11: note = source = /note="Description of Artificial Sequence: Synthetic peptide"
QSLVHTNGNTY

SEQ ID NO: 21 - DNA, 17 bp, synthetic construct
  misc_feature 1..17: note = source = /note="Description of Artificial Sequence: Synthetic primer"
  source 1..17: mol_type = other DNA
gtaaaacgacggccagt

SEQ ID NO: 22 - DNA, 972 bp, Mus sp.
  source 1..972: mol_type = genomic DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 23 - protein, 324 aa, Mus sp.
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 24 - DNA, 321 bp, Mus sp.
  source 1..321: mol_type = genomic DNA
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 25 - protein, 107 aa, Mus sp.
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 26 - DNA, 1332 bp, synthetic construct
  misc_feature 1..1332: note = source = /note="Description of Artificial Sequence: Synthetic polynucleotide"
  source 1..1332: mol_type = other DNA
gaagtgcagctggtggagtctgggggaggcttagtgaagcctggagggtccctgaaactctcctgtgcagcctctggattcgctttcagtagctatgacatgtcttgggttcgccagactccggagaagaggctggagtgggtcgcatacattagtcgtggtggtggtagcacctactatccagacactgtgaagggccgattcaccatctccagagacaatgccaagaacaccctgtacctgcaaatgagcagtctgaagtctgaggacacagccatgtattactgtggaagacatgctactacggcctactggtacttcgatgtctggggcgcagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 27 - protein, 444 aa, synthetic construct
  REGION 1..444: note = source = /note="Description of Artificial Sequence: Synthetic polypeptide"
EVQLVESGGGLVKPGGSLKLSCAASGFAFSSYDMSWVRQTPEKRLEWVAYISRGGGSTYYPDTVKGRFTISRDNAKNTLYLQMSSLKSEDTAMYYCGRHATTAYWYFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 28 - DNA, 657 bp, synthetic construct
  misc_feature 1..657: note = source = /note="Description of Artificial Sequence: Synthetic polynucleotide"
  source 1..657: mol_type = other DNA
gatgttgtgatgacccaaactccactctccctgcctgtcagtcttggagatcaagcctccatctcttgcagatctagtcagagccttgtacacactaatggcaacacctatttacattggtacctgcagaagccaggccagtctccaaaactcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 29 - protein, 219 aa, synthetic construct
  REGION 1..219: note = source = /note="Description of Artificial Sequence: Synthetic polypeptide"
DVVMTQTPLSLPVSLGDQASISCRSSQSLVHTNGNTYLHWYLQKPGQSPKLLIYKVSNRFSGVPDRFSGSGSGTDFTLKISRVEAEDLGVYFCSQSTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 30 - DNA, 18 bp, synthetic construct
  misc_feature 1..18: note = source = /note="Description of Artificial Sequence: Synthetic primer"
  source 1..18: mol_type = other DNA
caggaaacagctatgacc

SEQ ID NO: 31 - DNA, 360 bp, synthetic construct
  misc_feature 1..360: note = source = /note="Description of Artificial Sequence: Synthetic polynucleotide"
  source 1..360: mol_type = other DNA
gaggtacagcttgtcgagtcgggaggaggattggtaaaaccgggtgggtcactcaaattgtcgtgtgcggcgtcgggatttgcgttttcgtcgtatgatatgtcgtgggtgcgccagacgccggaaaaacgattggaatgggtcgcgtatatctcccgagggggaggttcgacatactatcccgacacggtcaaagggcgcttcacgatttcacgggacaatgcgaaaaacacgctttatcttcaaatgtcgtcgttgaaatcggaagataccgcgatgtattactgcgggaggcatgcgacgacggcgtattggtatttcgatgtgtggggagccggaacgacggtgacggtgtcgtcg

SEQ ID NO: 32 - DNA, 336 bp, synthetic construct
  misc_feature 1..336: note = source = /note="Description of Artificial Sequence: Synthetic polynucleotide"
  source 1..336: mol_type = other DNA
gacgtggtaatgacgcagacgccgttgtcccttcctgtctcgctcggagatcaggcgtcgatctcgtgtagaagctcgcagtcactcgtccataccaacgggaatacatatcttcactggtatttgcaaaagcccggacagtcaccgaagctcttgatctacaaagtatccaatcggttttcgggggtgcccgaccgattctcgggatcgggttcggggacggattttacgttgaagatttcgcgggtggaagcggaggatctcggtgtctacttttgttcgcagtcaacgcatgtcccgtggacgttcggaggcgggacaaaacttgagatcaag

SEQ ID NO: 33 - DNA, 360 bp, synthetic construct
  misc_feature 1..360: note = source = /note="Description of Artificial Sequence: Synthetic polynucleotide"
  source 1..360: mol_type = other DNA
gaagtacagttgttggagtcaggaggagggttggtccagccgggtggatcgttgcggctttcgtgtgcggcgtcgggattcgcgttttcatcgtatgacatgtcgtgggtgaggcaggcaccggggaaagggcttgaatgggtatcgtacatttcgagagggggaggatcgacgtattacccggatacggtgaaaggaaggtttacgatctcgcgcgacaattcaaagaatacgctttatcttcagatgaactcgctccgagcggaagatacggcggtatactattgcggtcgccatgcgacgacggcgtattggtatttcgatgtgtggggacaagggacgatggtcacggtgtcgtcg

SEQ ID NO: 34 - protein, 120 aa, synthetic construct
  REGION 1..120: note = source = /note="Description of Artificial Sequence: Synthetic polypeptide"
EVQLLESGGGLVQPGGSLRLSCAASGFAFSSYDMSWVRQAPGKGLEWVSYISRGGGSTYYPDTVKGRFTISRDNSKNTLYLQMNSLRAEDTAVYYCGRHATTAYWYFDVWGQGTMVTVSS

SEQ ID NO: 35 - DNA, 360 bp, synthetic construct
  misc_feature 1..360: note = source = /note="Description of Artificial Sequence: Synthetic polynucleotide"
  source 1..360: mol_type = other DNA
gaagtacagttgttggagtcaggaggagggttggtccagccgggtggatcgttgcggctttcgtgtgcggcgtcgggattcaccttttcacactatgacatgtcgtgggtgaggcaggcaccggggaaagggcttgaatgggtatcgtacatttcgagagggggaggatcgacgtattacccggattccgtgaaaggaaggtttacgatctcgcgcgacaattcaaagaatacgctttatcttcagatgaactcgctccgagcggaagatacggcggtatactattgcggtcgccatgcgacgacggcgtattggtatttcgatgtgtggggacaagggacgatggtcacggtgtcgtcg

SEQ ID NO: 36 - protein, 120 aa, synthetic construct
  REGION 1..120: note = source = /note="Description of Artificial Sequence: Synthetic polypeptide"
EVQLLESGGGLVQPGGSLRLSCAASGFTFSHYDMSWVRQAPGKGLEWVSYISRGGGSTYYPDSVKGRFTISRDNSKNTLYLQMNSLRAEDTAVYYCGRHATTAYWYFDVWGQGTMVTVSS

SEQ ID NO: 37 - DNA, 360 bp, synthetic construct
  misc_feature 1..360: note = source = /note="Description of Artificial Sequence: Synthetic polynucleotide"
  source 1..360: mol_type = other DNA
gaagtacagttgttggagtcaggaggagggttggtccagccgggtggatcgttgcggctttcgtgtgcggcgtcgggattcgcgttttcacactatgacatgtcgtgggtgaggcaggcaccggggaaagggcttgaatgggtatcgtacatttcgagagggggaggatcgacgtattacccggattccgtgaaaggaaggtttacgatctcgcgcgacaattcaaagaatacgctttatcttcagatgaactcgctccgagcggaagatacggcggtatactattgcggtcgccatgcgacgacggcgtattggtatttcgatgtgtggggacaagggacgatggtcacggtgtcgtcg

SEQ ID NO: 38 - protein, 120 aa, synthetic construct
  REGION 1..120: note = source = /note="Description of Artificial Sequence: Synthetic polypeptide"
EVQLLESGGGLVQPGGSLRLSCAASGFAFSHYDMSWVRQAPGKGLEWVSYISRGGGSTYYPDSVKGRFTISRDNSKNTLYLQMNSLRAEDTAVYYCGRHATTAYWYFDVWGQGTMVTVSS

SEQ ID NO: 39 - DNA, 360 bp, synthetic construct
  misc_feature 1..360: note = source = /note="Description of Artificial Sequence: Synthetic polynucleotide"
  source 1..360: mol_type = other DNA
gaagtacagttgttggagtcaggaggagggttggtccagccgggtggatcgttgcggctttcgtgtgcggcgtcgggattcaccttttcaaactatgacatgtcgtgggtgaggcaggcaccggggaaagggcttgaatgggtatcgtacatttcgagagggggaggatcgacgtattacccggattccgtgaaaggaaggtttacgatctcgcgcgacaattcaaagaatacgctttatcttcagatgaactcgctccgagcggaagatacggcggtatactattgcggtcgccatgcgacgacggcgtattggtatttcgatgtgtggggacaagggacgatggtcacggtgtcgtcg

SEQ ID NO: 40 - protein, 120 aa, synthetic construct
  REGION 1..120: note = source = /note="Description of Artificial Sequence: Synthetic polypeptide"
EVQLLESGGGLVQPGGSLRLSCAASGFTFSNYDMSWVRQAPGKGLEWVSYISRGGGSTYYPDSVKGRFTISRDNSKNTLYLQMNSLRAEDTAVYYCGRHATTAYWYFDVWGQGTMVTVSS

SEQ ID NO: 41 - DNA, 360 bp, synthetic construct
  misc_feature 1..360: note = source = /note="Description of Artificial Sequence: Synthetic polynucleotide"
  source 1..360: mol_type = other DNA
gaagtacagttgttggagtcaggaggagggttggtccagccgggtggatcgttgcggctttcgtgtgcggcgtcgggattcaccttttcatcgtatgacatgtcgtgggtgaggcaggcaccggggaaagggcttgaatgggtatcgtacatttcgagagggggaggatcgacgtattacccggattccgtgaaaggaaggtttacgatctcgcgcgacaattcaaagaatacgctttatcttcagatgaactcgctccgagcggaagatacggcggtatactattgcggtcgccatgcgacgacggcgtattggtatttcgatgtgtggggacaagggacgatggtcacggtgtcgtcg

SEQ ID NO: 42 - protein, 120 aa, synthetic construct
  REGION 1..120: note = source = /note="Description of Artificial Sequence: Synthetic polypeptide"
EVQLLESGGGLVQPGGSLRLSCAASGFTFSSYDMSWVRQAPGKGLEWVSYISRGGGSTYYPDSVKGRFTISRDNSKNTLYLQMNSLRAEDTAVYYCGRHATTAYWYFDVWGQGTMVTVSS

SEQ ID NO: 43 - DNA, 336 bp, synthetic construct
  misc_feature 1..336: note = source = /note="Description of Artificial Sequence: Synthetic polynucleotide"
  source 1..336: mol_type = other DNA
gatgtagtcatgacccaaacgccgctttcgttgtcggtgacgcccggacagcccgcgtcaatctcgtgtcggtcatcgcagtcgttggtacacacaaacggtaatacgtatctccattggtatctccagaagcccggccagtcgccgcagctcttgatctacaaagtgagcaatcgcttttcgggggtgccggatcggttctcgggatcggggtcaggaacggacttcacgcttaagatttcgagggtcgaggcggaggatgtcggagtctacttttgttcgcagtccacacatgtcccctggacgtttgggcaggggacgaaggtggaaatcaag

SEQ ID NO: 44 - protein, 112 aa, synthetic construct
  REGION 1..112: note = source = /note="Description of Artificial Sequence: Synthetic polypeptide"
DVVMTQTPLSLSVTPGQPASISCRSSQSLVHTNGNTYLHWYLQKPGQSPQLLIYKVSNRFSGVPDRFSGSGSGTDFTLKISRVEAEDVGVYFCSQSTHVPWTFGQGTKVEIK

SEQ ID NO: 45 - DNA, 336 bp, synthetic construct
  misc_feature 1..336: note = source = /note="Description of Artificial Sequence: Synthetic polynucleotide"
  source 1..336: mol_type = other DNA
gatgtagtcatgacccaaacgccgctttcgttgtcggtgacgcccggacagcccgcgtcaatctcgtgtcggtcatcgcagtcgttggtacacacacacggtaatacgtatctccattggtatctccagaagcccggccagtcgccgcagctcttgatctacaaagtgagcaatcgcttttcgggggtgccggatcggttctcgggatcggggtcaggaacggacttcacgcttaagatttcgagggtcgaggcggaggatgtcggagtctacttttgttcgcagtccacacatgtcccctggacgtttgggcaggggacgaaggtggaaatcaag

SEQ ID NO: 46 - protein, 112 aa, synthetic construct
  REGION 1..112: note = source = /note="Description of Artificial Sequence: Synthetic polypeptide"
DVVMTQTPLSLSVTPGQPASISCRSSQSLVHTHGNTYLHWYLQKPGQSPQLLIYKVSNRFSGVPDRFSGSGSGTDFTLKISRVEAEDVGVYFCSQSTHVPWTFGQGTKVEIK

SEQ ID NO: 47 - DNA, 336 bp, synthetic construct
  misc_feature 1..336: note = source = /note="Description of Artificial Sequence: Synthetic polynucleotide"
  source 1..336: mol_type = other DNA
gatgtagtcatgacccaaacgccgctttcgttgtcggtgacgcccggacagcccgcgtcaatctcgtgtcggtcatcgcagtcgttggtacacacacaaggtaatacgtatctccattggtatctccagaagcccggccagtcgccgcagctcttgatctacaaagtgagcaatcgcttttcgggggtgccggatcggttctcgggatcggggtcaggaacggacttcacgcttaagatttcgagggtcgaggcggaggatgtcggagtctacttttgttcgcagtccacacatgtcccctggacgtttgggcaggggacgaaggtggaaatcaag

SEQ ID NO: 48 - protein, 112 aa, synthetic construct
  REGION 1..112: note = source = /note="Description of Artificial Sequence: Synthetic polypeptide"
DVVMTQTPLSLSVTPGQPASISCRSSQSLVHTQGNTYLHWYLQKPGQSPQLLIYKVSNRFSGVPDRFSGSGSGTDFTLKISRVEAEDVGVYFCSQSTHVPWTFGQGTKVEIK

SEQ ID NO: 49 - DNA, 336 bp, synthetic construct
  misc_feature 1..336: note = source = /note="Description of Artificial Sequence: Synthetic polynucleotide"
  source 1..336: mol_type = other DNA
gatgtagtcatgacccaaacgccgctttcgttgtcggtgacgcccggacagcccgcgtcaatctcgtgtcggtcatcgcagtcgttggtacacacatacggtaatacgtatctccattggtatctccagaagcccggccagtcgccgcagctcttgatctacaaagtgagcaatcgcttttcgggggtgccggatcggttctcgggatcggggtcaggaacggacttcacgcttaagatttcgagggtcgaggcggaggatgtcggagtctacttttgttcgcagtccacacatgtcccctggacgtttgggcaggggacgaaggtggaaatcaag

SEQ ID NO: 50 - protein, 112 aa, synthetic construct
  REGION 1..112: note = source = /note="Description of Artificial Sequence: Synthetic polypeptide"
DVVMTQTPLSLSVTPGQPASISCRSSQSLVHTYGNTYLHWYLQKPGQSPQLLIYKVSNRFSGVPDRFSGSGSGTDFTLKISRVEAEDVGVYFCSQSTHVPWTFGQGTKVEIK

SEQ ID NO: 51 - protein, 5 aa, synthetic construct
  REGION 1..5: note = source = /note="Description of Artificial Sequence: Synthetic peptide"
HYDMS

SEQ ID NO: 52 - protein, 5 aa, synthetic construct
  REGION 1..5: note = source = /note="Description of Artificial Sequence: Synthetic peptide"
NYDMS

SEQ ID NO: 53 - protein, 17 aa, synthetic construct
  REGION 1..17: note = source = /note="Description of Artificial Sequence: Synthetic peptide"
YISRGGGSTYYPDSVKG

SEQ ID NO: 54 - protein, 7 aa, synthetic construct
  REGION 1..7: note = source = /note="Description of Artificial Sequence: Synthetic peptide"
GFTFSHY

SEQ ID NO: 55 - protein, 7 aa, synthetic construct
  REGION 1..7: note = source = /note="Description of Artificial Sequence: Synthetic peptide"
GFAFSHY

SEQ ID NO: 56 - protein, 7 aa, synthetic construct
  REGION 1..7: note = source = /note="Description of Artificial Sequence: Synthetic peptide"
GFTFSNY

SEQ ID NO: 57 - protein, 7 aa, synthetic construct
  REGION 1..7: note = source = /note="Description of Artificial Sequence: Synthetic peptide"
GFTFSSY

SEQ ID NO: 58 - protein, 8 aa, synthetic construct
  REGION 1..8: note = source = /note="Description of Artificial Sequence: Synthetic peptide"
GFTFSHYD

SEQ ID NO: 59 - protein, 8 aa, synthetic construct
  REGION 1..8: note = source = /note="Description of Artificial Sequence: Synthetic peptide"
GFAFSHYD

SEQ ID NO: 60 - protein, 8 aa, synthetic construct
  REGION 1..8: note = source = /note="Description of Artificial Sequence: Synthetic peptide"
GFTFSNYD

SEQ ID NO: 61 - protein, 8 aa, synthetic construct
  REGION 1..8: note = source = /note="Description of Artificial Sequence: Synthetic peptide"
GFTFSSYD

SEQ ID NO: 62 - protein, 16 aa, synthetic construct
  REGION 1..16: note = source = /note="Description of Artificial Sequence: Synthetic peptide"
RSSQSLVHTHGNTYLH

SEQ ID NO: 63 - protein, 16 aa, synthetic construct
  REGION 1..16: note = source = /note="Description of Artificial Sequence: Synthetic peptide"
RSSQSLVHTQGNTYLH

SEQ ID NO: 64 - protein, 16 aa, synthetic construct
  REGION 1..16: note = source = /note="Description of Artificial Sequence: Synthetic peptide"
RSSQSLVHTYGNTYLH

SEQ ID NO: 65 - protein, 11 aa, synthetic construct
  REGION 1..11: note = source = /note="Description of Artificial Sequence: Synthetic peptide"
QSLVHTHGNTY

SEQ ID NO: 66 - protein, 11 aa, synthetic construct
  REGION 1..11: note = source = /note="Description of Artificial Sequence: Synthetic peptide"
QSLVHTQGNTY

SEQ ID NO: 67 - protein, 11 aa, synthetic construct
  REGION 1..11: note = source = /note="Description of Artificial Sequence: Synthetic peptide"
QSLVHTYGNTY

SEQ ID NO: 68 - DNA, 990 bp, Homo sapiens
  source 1..990: mol_type = genomic DNA
gcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 69 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 321 bp, Homo sapiens
  source 1..321: mol_type = genomic DNA
cgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 71 - DNA, 321 bp, Homo sapiens
  source 1..321: mol_type = genomic DNA
cgcacagttgcagcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 72 - protein, 107 aa, Homo sapiens [sequence omitted]

SEQ ID NO: 73 - DNA, 1350 bp, synthetic construct
  misc_feature 1..1350: note = source = /note="Description of Artificial Sequence: Synthetic polynucleotide"
  source 1..1350: mol_type = other DNA
gaggtacagcttgtcgagtcgggaggaggattggtaaaaccgggtgggtcactcaaattgtcgtgtgcggcgtcgggatttgcgttttcgtcgtatgatatgtcgtgggtgcgccagacgccggaaaaacgattggaatgggtcgcgtatatctcccgagggggaggttcgacatactatcccgacacggtcaaagggcgcttcacgatttcacgggacaatgcgaaaaacacgctttatcttcaaatgtcgtcgttgaaatcggaagataccgcgatgtattactgcgggaggcatgcgacgacggcgtattggtatttcgatgtgtggggagccggaacgacggtgacggtgtcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 74 - protein, 450 aa, synthetic construct
  REGION 1..450: note = source = /note="Description of Artificial Sequence: Synthetic polypeptide"
EVQLVESGGGLVKPGGSLKLSCAASGFAFSSYDMSWVRQTPEKRLEWVAYISRGGGSTYYPDTVKGRFTISRDNAKNTLYLQMSSLKSEDTAMYYCGRHATTAYWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - DNA, 1350 bp, synthetic construct
  misc_feature 1..1350: note = source = /note="Description of Artificial Sequence: Synthetic polynucleotide"
  source 1..1350: mol_type = other DNA
gaagtacagttgttggagtcaggaggagggttggtccagccgggtggatcgttgcggctttcgtgtgcggcgtcgggattcgcgttttcatcgtatgacatgtcgtgggtgaggcaggcaccggggaaagggcttgaatgggtatcgtacatttcgagagggggaggatcgacgtattacccggatacggtgaaaggaaggtttacgatctcgcgcgacaattcaaagaatacgctttatcttcagatgaactcgctccgagcggaagatacggcggtatactattgcggtcgccatgcgacgacggcgtattggtatttcgatgtgtggggacaagggacgatggtcacggtgtcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 76 - protein, 450 aa, synthetic construct
  REGION 1..450: note = source = /note="Description of Artificial Sequence: Synthetic polypeptide"
EVQLLESGGGLVQPGGSLRLSCAASGFAFSSYDMSWVRQAPGKGLEWVSYISRGGGSTYYPDTVKGRFTISRDNSKNTLYLQMNSLRAEDTAVYYCGRHATTAYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - DNA, 1350 bp, synthetic construct
  misc_feature 1..1350: note = source = /note="Description of Artificial Sequence: Synthetic polynucleotide"
  source 1..1350: mol_type = other DNA
gaagtacagttgttggagtcaggaggagggttggtccagccgggtggatcgttgcggctttcgtgtgcggcgtcgggattcaccttttcacactatgacatgtcgtgggtgaggcaggcaccggggaaagggcttgaatgggtatcgtacatttcgagagggggaggatcgacgtattacccggattccgtgaaaggaaggtttacgatctcgcgcgacaattcaaagaatacgctttatcttcagatgaactcgctccgagcggaagatacggcggtatactattgcggtcgccatgcgacgacggcgtattggtatttcgatgtgtggggacaagggacgatggtcacggtgtcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 78 - protein, 450 aa, synthetic construct
  REGION 1..450: note = source = /note="Description of Artificial Sequence: Synthetic polypeptide"
EVQLLESGGGLVQPGGSLRLSCAASGFTFSHYDMSWVRQAPGKGLEWVSYISRGGGSTYYPDSVKGRFTISRDNSKNTLYLQMNSLRAEDTAVYYCGRHATTAYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - DNA, 1350 bp, synthetic construct
  misc_feature 1..1350: note = source = /note="Description of Artificial Sequence: Synthetic polynucleotide"
  source 1..1350: mol_type = other DNA
gaagtacagttgttggagtcaggaggagggttggtccagccgggtggatcgttgcggctttcgtgtgcggcgtcgggattcgcgttttcacactatgacatgtcgtgggtgaggcaggcaccggggaaagggcttgaatgggtatcgtacatttcgagagggggaggatcgacgtattacccggattccgtgaaaggaaggtttacgatctcgcgcgacaattcaaagaatacgctttatcttcagatgaactcgctccgagcggaagatacggcggtatactattgcggtcgccatgcgacgacggcgtattggtatttcgatgtgtggggacaagggacgatggtcacggtgtcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 80 - protein, 450 aa, synthetic construct
  REGION 1..450: note = source = /note="Description of Artificial Sequence: Synthetic polypeptide"
EVQLLESGGGLVQPGGSLRLSCAASGFAFSHYDMSWVRQAPGKGLEWVSYISRGGGSTYYPDSVKGRFTISRDNSKNTLYLQMNSLRAEDTAVYYCGRHATTAYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 1350 bp, synthetic construct
  misc_feature 1..1350: note = source = /note="Description of Artificial Sequence: Synthetic polynucleotide"
  source 1..1350: mol_type = other DNA
gaagtacagttgttggagtcaggaggagggttggtccagccgggtggatcgttgcggctttcgtgtgcggcgtcgggattcaccttttcaaactatgacatgtcgtgggtgaggcaggcaccggggaaagggcttgaatgggtatcgtacatttcgagagggggaggatcgacgtattacccggattccgtgaaaggaaggtttacgatctcgcgcgacaattcaaagaatacgctttatcttcagatgaactcgctccgagcggaagatacggcggtatactattgcggtcgccatgcgacgacggcgtattggtatttcgatgtgtggggacaagggacgatggtcacggtgtcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 82 - protein, 450 aa, synthetic construct
  REGION 1..450: note = source = /note="Description of Artificial Sequence: Synthetic polypeptide"
EVQLLESGGGLVQPGGSLRLSCAASGFTFSNYDMSWVRQAPGKGLEWVSYISRGGGSTYYPDSVKGRFTISRDNSKNTLYLQMNSLRAEDTAVYYCGRHATTAYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - DNA, 1350 bp, synthetic construct
  misc_feature 1..1350: note = source = /note="Description of Artificial Sequence: Synthetic polynucleotide"
  source 1..1350: mol_type = other DNA
gaagtacagttgttggagtcaggaggagggttggtccagccgggtggatcgttgcggctttcgtgtgcggcgtcgggattcaccttttcatcgtatgacatgtcgtgggtgaggcaggcaccggggaaagggcttgaatgggtatcgtacatttcgagagggggaggatcgacgtattacccggattccgtgaaaggaaggtttacgatctcgcgcgacaattcaaagaatacgctttatcttcagatgaactcgctccgagcggaagatacggcggtatactattgcggtcgccatgcgacgacggcgtattggtatttcgatgtgtggggacaagggacgatggtcacggtgtcgtcggcctcaacaaaaggaccaagtgtgttcccactcgcccctagcagcaagagtacatccgggggcactgcagcactcggctgcctcgtcaaggattattttccagagccagtaaccgtgagctggaacagtggagcactcacttctggtgtccatacttttcctgctgtcctgcaaagctctggcctgtactcactcagctccgtcgtgaccgtgccatcttcatctctgggcactcagacctacatctgtaatgtaaaccacaagcctagcaatactaaggtcgataagcgggtggaacccaagagctgcgacaagactcacacttgtcccccatgccctgcccctgaacttctgggcggtcccagcgtctttttgttcccaccaaagcctaaagatactctgatgataagtagaacacccgaggtgacatgtgttgttgtagacgtttcccacgaggacccagaggttaagttcaactggtacgttgatggagtcgaagtacataatgctaagaccaagcctagagaggagcagtataatagtacataccgtgtagtcagtgttctcacagtgctgcaccaagactggctcaacggcaaagaatacaaatgcaaagtgtccaacaaagcactcccagcccctatcgagaagactattagtaaggcaaaggggcagcctcgtgaaccacaggtgtacactctgccacccagtagagaggaaatgacaaagaaccaagtctcattgacctgcctggtgaaaggcttctaccccagcgacatcgccgttgagtgggagagtaacggtcagcctgagaacaattacaagacaacccccccagtgctggatagtgacgggtctttctttctgtacagtaagctgactgtggacaagtcccgctggcagcagggtaacgtcttcagctgttccgtgatgcacgaggcattgcacaaccactacacccagaagtcactgagcctgagcccagggaag

SEQ ID NO: 84 - protein, 450 aa, synthetic construct
  REGION 1..450: note = source = /note="Description of Artificial Sequence: Synthetic polypeptide"
EVQLLESGGGLVQPGGSLRLSCAASGFTFSSYDMSWVRQAPGKGLEWVSYISRGGGSTYYPDSVKGRFTISRDNSKNTLYLQMNSLRAEDTAVYYCGRHATTAYWYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 657 bp, synthetic construct
  misc_feature 1..657: note = source = /note="Description of Artificial Sequence: Synthetic polynucleotide"
  source 1..657: mol_type = other DNA
gacgtggtaatgacgcagacgccgttgtcccttcctgtctcgctcggagatcaggcgtcgatctcgtgtagaagctcgcagtcactcgtccataccaacgggaatacatatcttcactggtatttgcaaaagcccggacagtcaccgaagctcttgatctacaaagtatccaatcggttttcgggggtgcccgaccgattctcgggatcgggttcggggacggattttacgttgaagatttcgcgggtggaagcggaggatctcggtgtctacttttgttcgcagtcaacgcatgtcccgtggacgttcggaggcgggacaaaacttgagatcaagcgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 86 - protein, 219 aa, synthetic construct
  REGION 1..219: note = source = /note="Description of Artificial Sequence: Synthetic polypeptide"
DVVMTQTPLSLPVSLGDQASISCRSSQSLVHTNGNTYLHWYLQKPGQSPKLLIYKVSNRFSGVPDRFSGSGSGTDFTLKISRVEAEDLGVYFCSQSTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - DNA, 657 bp, synthetic construct
  misc_feature 1..657: note = source = /note="Description of Artificial Sequence: Synthetic polynucleotide"
  source 1..657: mol_type = other DNA
gatgtagtcatgacccaaacgccgctttcgttgtcggtgacgcccggacagcccgcgtcaatctcgtgtcggtcatcgcagtcgttggtacacacaaacggtaatacgtatctccattggtatctccagaagcccggccagtcgccgcagctcttgatctacaaagtgagcaatcgcttttcgggggtgccggatcggttctcgggatcggggtcaggaacggacttcacgcttaagatttcgagggtcgaggcggaggatgtcggagtctacttttgttcgcagtccacacatgtcccctggacgtttgggcaggggacgaaggtggaaatcaagcgcacagttgctgcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 88 - protein, 219 aa, synthetic construct
  REGION 1..219: note = source = /note="Description of Artificial Sequence: Synthetic polypeptide"
DVVMTQTPLSLSVTPGQPASISCRSSQSLVHTNGNTYLHWYLQKPGQSPQLLIYKVSNRFSGVPDRFSGSGSGTDFTLKISRVEAEDVGVYFCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - DNA, 657 bp, synthetic construct
  misc_feature 1..657: note = source = /note="Description of Artificial Sequence: Synthetic polynucleotide"
  source 1..657: mol_type = other DNA
gatgtagtcatgacccaaacgccgctttcgttgtcggtgacgcccggacagcccgcgtcaatctcgtgtcggtcatcgcagtcgttggtacacacacacggtaatacgtatctccattggtatctccagaagcccggccagtcgccgcagctcttgatctacaaagtgagcaatcgcttttcgggggtgccggatcggttctcgggatcggggtcaggaacggacttcacgcttaagatttcgagggtcgaggcggaggatgtcggagtctacttttgttcgcagtccacacatgtcccctggacgtttgggcaggggacgaaggtggaaatcaagcgcacagttgcagcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 90 - protein, 219 aa, synthetic construct
  REGION 1..219: note = source = /note="Description of Artificial Sequence: Synthetic polypeptide"
DVVMTQTPLSLSVTPGQPASISCRSSQSLVHTHGNTYLHWYLQKPGQSPQLLIYKVSNRFSGVPDRFSGSGSGTDFTLKISRVEAEDVGVYFCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - DNA, 657 bp, synthetic construct
  misc_feature 1..657: note = source = /note="Description of Artificial Sequence: Synthetic polynucleotide"
  source 1..657: mol_type = other DNA
gatgtagtcatgacccaaacgccgctttcgttgtcggtgacgcccggacagcccgcgtcaatctcgtgtcggtcatcgcagtcgttggtacacacacaaggtaatacgtatctccattggtatctccagaagcccggccagtcgccgcagctcttgatctacaaagtgagcaatcgcttttcgggggtgccggatcggttctcgggatcggggtcaggaacggacttcacgcttaagatttcgagggtcgaggcggaggatgtcggagtctacttttgttcgcagtccacacatgtcccctggacgtttgggcaggggacgaaggtggaaatcaagcgcacagttgcagcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 92 - protein, 219 aa, synthetic construct
  REGION 1..219: note = source = /note="Description of Artificial Sequence: Synthetic polypeptide"
DVVMTQTPLSLSVTPGQPASISCRSSQSLVHTQGNTYLHWYLQKPGQSPQLLIYKVSNRFSGVPDRFSGSGSGTDFTLKISRVEAEDVGVYFCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - DNA, 657 bp, synthetic construct
  misc_feature 1..657: note = source = /note="Description of Artificial Sequence: Synthetic polynucleotide"
  source 1..657: mol_type = other DNA
gatgtagtcatgacccaaacgccgctttcgttgtcggtgacgcccggacagcccgcgtcaatctcgtgtcggtcatcgcagtcgttggtacacacatacggtaatacgtatctccattggtatctccagaagcccggccagtcgccgcagctcttgatctacaaagtgagcaatcgcttttcgggggtgccggatcggttctcgggatcggggtcaggaacggacttcacgcttaagatttcgagggtcgaggcggaggatgtcggagtctacttttgttcgcagtccacacatgtcccctggacgtttgggcaggggacgaaggtggaaatcaagcgcacagttgcagcccccagcgtgttcattttcccacctagcgatgagcagctgaaaagcggtactgcctctgtcgtatgcttgctcaacaacttttacccacgtgaggctaaggtgcagtggaaagtggataatgcacttcaatctggaaacagtcaagagtccgtgacagaacaggacagcaaagactcaacttattcactctcttccaccctgactctgtccaaggcagactatgaaaaacacaaggtatacgcctgcgaggttacacaccagggtttgtctagtcctgtcaccaagtccttcaataggggcgaatgt

SEQ ID NO: 94 - protein, 219 aa, synthetic construct
  REGION 1..219: note = source = /note="Description of Artificial Sequence: Synthetic polypeptide"
DVVMTQTPLSLSVTPGQPASISCRSSQSLVHTYGNTYLHWYLQKPGQSPQLLIYKVSNRFSGVPDRFSGSGSGTDFTLKISRVEAEDVGVYFCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 1604 aa, Homo sapiens
APPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPL

SEQ ID NO: 96 - protein, 428 aa, Homo sapiens
APPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCE